SEQUENCE LISTING (30 sequences)

SEQ ID NO: 1 - DNA, 52 bp, Artificial
  protospacer
  misc_feature (1)..(36): n is a, c, g, or t
  misc_feature (39)..(52): n is a, c, g, or t
nnnnnnnnnnnnnnnnnnnnnnnnnnnnnnnnnnnnggnnnnnnnnnnnnnn

SEQ ID NO: 2 - DNA, 52 bp, Artificial
  protospacer
  misc_feature (1)..(14): n is a, c, g, or t
  misc_feature (17)..(52): n is a, c, g, or t
nnnnnnnnnnnnnnccnnnnnnnnnnnnnnnnnnnnnnnnnnnnnnnnnnnn

SEQ ID NO: 3 - DNA, 99 bp, Artificial
  spacer and guide RNA
  misc_feature (1)..(20): n is a, c, g, t or u
nnnnnnnnnnnnnnnnnnnnguuuuagagcuagaaauagcaaguuaaaauaaggcuaguccguuaucaacuugaaaaaguggcaccgagucgguguuuu

SEQ ID NO: 4 - protein, 1368 aa, Streptococcus pyogenes [sequence omitted]

SEQ ID NO: 5 - DNA, 92 bp, Artificial
  primer and gblock sequence
tacgaaccgggtaatactaagtgattgactcttgctgaccttttattaagaactaaatggtctttgaaaagataatgtatgattatgctttc

SEQ ID NO: 6 - DNA, 87 bp, Artificial
  primer and gblock sequence
taataagtgatcttatgtatgaaattcttaaaaaaggacacctgtaagcgttgatttctaagacataaaaaacaaaaaaagcaccac

SEQ ID NO: 7 - DNA, 89 bp, Artificial
  primer and gblock sequence
tacgaaccgggtaatactaagtgattgactcttgctgaccttttattaagaactaaatggagacataaaaaacaaaaaaagcaccaccg

SEQ ID NO: 8 - DNA, 87 bp, Artificial
  primer and gblock sequence
taataagtgatcttatgtatgaaattcttaaaaaaggacacctgtaagcgttgatttctatcgacactggatggcggcgttagtatc

SEQ ID NO: 9 - DNA, 863 bp, Artificial
  primer and gblock sequence
agccagatgccattcaacaagttcttcgtgcaggagataccaaagtggatagaacgtttcagcccaaagatgcgtgaggggcttcagcgtagcgacgggcgttacggggtggagggtgacgagaaagaggctgctagctacttttacacgatgttcgcttttagaaaagttaagcaatacatagagcctgagtcagttaaaccaaattgaacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtatgaccgaccaagcgacgcccaacctgccatcacgagatttcgatcccaccgccgccttctatgaaaggtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctgaaggggatgaaaaggaaggttttagagctagaaatagcaagttaaaataaggctagtccgttatcaacttgaaaaagtggcaccgagtcggtggtgctttttttgttttttatgtct

SEQ ID NO: 10 - DNA, 84 bp, Artificial
  primer and gblock sequence
tatgttgtgccattcgaaaccttaagaagtttggctgatgaatacggtttggaactagtaagccagatgccattcaacaagttc

SEQ ID NO: 11 - DNA, 88 bp, Artificial
  primer and gblocker sequence
gtaatacggccgaaatacagatgctttatagtagggttattgtttctattcatttttattagacataaaaaacaaaaaaagcaccacc

SEQ ID NO: 12 - DNA, 388 bp, Artificial
  primer and gblocker sequence
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcacttgaagattctttagtgtgttttagagctagaaatagcaagttaaaataaggctagtccgttatcaacttgaaaaagtggcaccgagtcggtggtgctttttttgttttttatgtct

SEQ ID NO: 13 - DNA, 90 bp, Artificial
  primer and gblocker sequence
tgatgtgctaacgattgagattgagcatgttgatgttcctaccctgaaaaacctgcaagtaaaacatcccaaattaaaaatttacccttc

SEQ ID NO: 14 - DNA, 90 bp, Artificial
  primer and gblocker sequence
gaagggtaaatttttaatttgggatgttttacttgcaggtttttcagggtaggaacatcaacatgctcaatctcaatcgttagcacatca

SEQ ID NO: 15 - DNA, 30 bp, Artificial
  primer and gblocker sequence
atagataatgttcctgaatatgttgtgcca

SEQ ID NO: 16 - DNA, 32 bp, Artificial
  primer and gblock sequence
ttactacatatagaagtcttgtaatacggccg

SEQ ID NO: 17 - DNA, 29 bp, Artificial
  primer and gblock sequence
gctacgaaccgggtaatactaagtgattg

SEQ ID NO: 18 - DNA, 31 bp, Artificial
  primer and gblock sequence
caggtaattattccttgcttcttgttactgg

SEQ ID NO: 19 - DNA, 31 bp, Artificial
  primer
tacgaaccgggtaatactaagtgattgactc

SEQ ID NO: 20 - DNA, 23 bp, Artificial
  primer
cgctagcccaagagggcactaca

SEQ ID NO: 21 - DNA, 34 bp, Artificial
  primer
taccaactgttctagaatccatacttgattgttt

SEQ ID NO: 22 - DNA, 31 bp, Artificial; identical to SEQ ID NO: 19
  primer

SEQ ID NO: 23 - DNA, 32 bp, Artificial
  primer
cctcctaatataccaactgttctagaatccat

SEQ ID NO: 24 - DNA, 30 bp, Artificial
  primer
aaacttctccgcagtgaaagataaatgatc

SEQ ID NO: 25 - DNA, 30 bp, Artificial
  primer
cgaggagccgttcaatttggtttaactgac

SEQ ID NO: 26 - DNA, 21 bp, Artificial
  primer
agatgcgtgaggggcttcagc

SEQ ID NO: 27 - DNA, 20 bp, Artificial
  primer
gaaggggatgaaaaggaagc

SEQ ID NO: 28 - DNA, 20 bp, Artificial
  primer
cgctttccggttcgatatac

SEQ ID NO: 29 - DNA, 25 bp, Artificial
  primer
cgaaagattcagagccccagaagct

SEQ ID NO: 30 - DNA, 26 bp, Artificial
  primer
cggtgatttccttttgcattctttcg

(19) United States
(12) Patent Application Publication
(10) Pub. No.: US 2019/0175762 A1
(43) Pub. Date: Jun. 13, 2019

(54) Dependent Component Genome Editing Gene Drives

(71) Applicant: President and Fellows of Harvard College, Cambridge, MA (US)

(72) Inventors: Kevin M. Esvelt, Auburndale, MA (US); Jianghong Min, Boston, MA (US)

(73) Assignee: President and Fellows of Harvard College, Cambridge, MA (US)

(21) Appl. No.: 15/764,949

(22) PCT Filed: Sep. 28, 2016
(86) PCT No.: PCT/US16/54059

§ 371 Date: Mar. 30, 2018

Related U.S. Application Data
(60) Provisional application No. 62/236,545, filed on Oct. 2, 2015.

(51) Int. Cl. A61K 48/00

(52) U.S. Cl. CPC A61K 48/0058; A61K 48/0008; A61P 31/12; A61K 48/0091; A61K 48/0066

(57) ABSTRACT

Dependent gene drive systems and their use in genome editing are disclosed. Methods of making a genome editing system using dependent gene drives are also disclosed.

### RELATED APPLICATION DATA

This application claims priority to U.S. Provisional Application No. 62/236,545 filed on Oct. 2, 2015 which is hereby incorporated herein by reference in its entirety for all purposes.

### BACKGROUND

Gene drives are generally known as genetic elements that skew the natural odds in their favor of being inherited and passed on by progeny. Examples include homing endonuclease genes that copy themselves into chromosomes lacking them, segregation distorters that destroy competing chromosomes during meiosis, transposons that insert copies of themselves elsewhere in the genome, Medea elements that eliminate competing siblings who do not inherit them, and maternally heritable microorganisms such as Wolbachia that induce cytoplasmic incompatibility to favor the spread of infected individuals. Because they circumvent the normal rules of natural selection, all of these elements have been considered as potential “gene drive” systems capable of spreading engineered modifications through insect vector populations to block the spread of disease. Homing endonuclease based gene drives have been proposed as a means of genetically controlling malaria mosquito populations. See Windbichler et al., Nature, doi:10.1038/nature09937 (2011). Site-specific selfish genes have been proposed as tools for the control and genetic engineering of natural populations. See Burt, Proc. R. Soc. Lond. B (2003) 270, 921-928 (2003). However, such proposed gene drives are limited in their site specificity or difficult to express in various organisms. A need therefore exists to develop gene drives which can target any desired gene and can be utilized across a broad spectrum of organisms.

### SUMMARY

Aspects of the present disclosure are directed to RNA guided gene drives, and in particular, a foreign nucleic acid sequence which is stably introduced into a germline cell of a desired organism. According to certain aspects, a genome editing system includes two or more or a plurality of components which, when present in the cell, cooperate or combine to result in editing of a target nucleic acid sequence. When all of the components necessary to result in genome editing and to act as a gene drive are provided on the same foreign nucleic acid sequence inserted into the genome of the cell, the foreign nucleic acid sequence can cut the target locus and the cut can be repaired using the foreign nucleic acid sequence as a template making the cell homologous for the foreign nucleic acid sequence, as the gene drive concept is described herein. As a gene drive, the genome editing system can be driven through essentially unlimited progeny or offspring.

Aspects of the present disclosure are directed to limiting progression of a genome editing system through progeny to a finite Nth generation, whereby the genome editing system can be contained within a local population. According to this aspect, a genome editing system within a foreign nucleic acid sequence is dependent upon expression of a molecule or molecules which then promote the foreign nucleic acid sequence to cut the target locus and the cut can be repaired using the foreign nucleic acid sequence as a template making the cell homologous for the foreign nucleic acid sequence. According to this aspect, the foreign nucleic acid sequence, when expressed, lacks the capability of functioning as a gene drive, i.e. to relicate itself at a target cut site. Instead, it is dependent upon the presence of a gene drive initiator molecule or molecules which promotes the replication of the foreign nucleic acid at a target locus, such as by promoting cutting at the target locus so that the foreign nucleic acid can then be inserted at the cut site, such as by homologous recombination. According to this aspect, separate foreign nucleic acid sequences encoding each component of the genome editing system or a subset of the separate components of the genome editing system are located given distances away from each other on the same chromosome or on different chromosomes, only a component or group of components of the genome editing system and not the entire genome editing system can be placed under the influence of an initiator molecule or molecules to achieve the component or group of components being a gene drive. Only those components or group of components of the genome editing system dependent on the initiator molecule or molecules can be replicated into a target locus as a gene drive, while other components of the genome editing system cannot function as a gene drive. In this manner, where only some of the components of the genome editing system are capable of being a gene drive since only some of the components are dependent upon an initiator molecule or molecules which may be the same or different, proliferation of the genome editing system through generations of progeny is limited. According to these aspects, the genome editing system can ultimately cut and insert a cargo gene of interest at a target locus in a limited number of generations of progeny thereby creating a limited number of progeny with the desired phenotype.

According to one aspect, different components of the genome editing system are dependent upon a different initiator molecule or molecules enabling such a component to be a gene drive. According to one aspect, each component or subcomponent of a genome editing system can have an associated initiator molecule or molecules. According to one aspect, the initiator molecule or molecules which promote the foreign nucleic acid sequence being a gene drive are not included within the foreign nucleic acid sequence. The foreign nucleic acid is thereby dependent on the separately encoded initiator molecule or molecules to function as a gene drive. According to one aspect, two or more or a plurality of foreign nucleic acid sequences can be inserted into a genome of a cell, such as a germline cell, where one of the two or more or plurality of foreign nucleic acids encodes the initiator molecule or molecules for another one of the two or more or plurality of foreign nucleic acids. In this manner, expression of a first nucleic acid sequence to produce a first initiator molecule or molecules allows a second nucleic acid sequence to function as a gene drive. Expression of the second nucleic acid sequence to produce a second initiator molecule or molecules allows a third nucleic acid sequence to function as a gene drive. Expression of the third nucleic acid sequence to produce a third initiator molecule or molecules allows a fourth nucleic acid sequence to function as a gene drive. Accordingly, expression of an Nth nucleic acid sequence to produce a Nth molecule or molecules allows an Nth+1 nucleic acid sequence to function as a gene drive. In this manner, the sequence of expression of nucleic acid sequences can be thought of as a chain, with expression of the first nucleic acid sequence allowing the second nucleic acid sequence to function as a gene drive, while expression of the second nucleic acid sequence allowing the third nucleic acid sequence to function as a gene drive and so on. According to this aspect, the ability of a particular nucleic acid sequence (the Nth nucleic acid sequence, for example) to function as a gene drive depends on the expression of the initiator molecule or molecules by the preceding nucleic acid sequence (i.e., the Nth-1 nucleic acid sequence).

Accordingly, aspects of the present disclosure are directed to a method of designing a chain of mutually dependent nucleic acid sequences that when expressed exhibit a genome editing system capable of rendering a cell, such as a germline cell, homozygous for a desired cargo gene, but inheritance will be biased for a fixed or given number of generations. In this manner, the genome modification will be limited to a local population of organisms and will not spread indefinitely. According to certain aspects, components of a genome editing system can be separated within a genome such that one or more components do not exhibit gene drive. According to this aspect, the genome editing system providing the cargo gene and the associate phenotype cannot be cannot drive through populations indefinitely.

Aspects of the present disclosure are directed to altering the genome of a cell, such as a germline cell, to locate one or more components of a genome editing system at different locations on the genome such that only one or more portions of the genome editing system are capable of being a gene drive. In this manner, the one or more components of the genome editing system rely upon the expression of a separate molecule, such as an initiator molecule or molecules which then facilitates operation of a gene drive. According to one aspect, the components of a genome editing system are all located on the same foreign nucleic acid sequence, but operation of the foreign nucleic acid sequence as a gene drive is dependent upon the expression of one or more initiator molecules.

According to certain aspects, the genome editing system is a Cas/guide RNA genome editing system as described herein. The nucleic acid sequences encoding the Cas portion and the guide RNA portion and the cargo gene portion for a given target locus, may be separated as described herein and under the influence of an initiator molecule or molecules, such as a different guide RNA or guide RNAs targeting a different locus. Accordingly, only the nucleic acid encoding the guide RNA may function as a gene drive. Accordingly, only the Cas may function as a gene drive. For a given target locus at which a cargo gene is to be inserted, the Cas and the guide RNA for that given target locus may each separately function as gene drives because of being dependent on a guide RNA or guide RNAs for a different target locus.

The term foreign nucleic acid sequence as described herein and the term RNA guided gene drive may be used interchangeable herein. A resulting transgenic organism may be developed from the germline cell. The resulting transgenic organism can then be introduced into a wild type population, and through mating by the transgenic organism with a wild type organism, the foreign nucleic acid sequence is transferred to resulting offspring or progeny. As a result, methods of the present disclosure are directed to the production of a population of transgenic organisms having desired traits from an initial transgenic organism and a wild type organism. When the transgenic organism is introduced into a wild type population and mates with a wild type organism, the resulting progeny may be referred to as an altered wild type population. As a result of the foreign nucleic acid sequence being stably entered into the genome of the transgenic offspring or transgenic progeny, transgenic offspring or transgenic progeny may have one or more desired traits resulting from expression of the foreign nucleic acid. According to certain aspects, methods described herein may be used to create an altered wild type population of transgenic organisms where the transgenic organisms exhibit one or more desired traits resulting from the expression of the foreign nucleic acid.

According to certain aspects, the foreign nucleic acid sequence encodes at least an RNA guided DNA binding protein, such as one or more of an RNA guided DNA binding protein nuclease, an RNA guided DNA binding protein nickase or a nuclease null RNA guided DNA binding protein, and one or more or a plurality of guide RNAs (ribonucleic acids). A guide RNA is complementary to DNA (deoxyribonucleic acid), such as a target DNA in the genome of a germline cell. The foreign nucleic acid sequence also encodes at least one or more promoters such that the germline cell may express the RNA guided DNA binding protein and the guide RNAs or any other nucleic acid sequence or gene which may be in the foreign nucleic acid sequence. One of skill will readily be able to identify suitable promoters based on the present disclosure and the particular germline cell. The foreign nucleic acid sequence may also include any other nucleic acid sequence or sequences known to those of skill in the art to be required for expression of the foreign nucleic acid sequence by a germline cell. The foreign nucleic acid sequence may also include any other gene sequence or gene sequences desired to be expressed by the germline cell. Such a gene sequence or such gene sequences may be referred to as “cargo DNA.” It is to be understood that one of skill will readily be able to identify one or more gene sequences depending upon the desired trait one of skill wishes to be exhibited by the germline cell or the organism developed from the germline cell when the cell expresses the foreign nucleic acid sequence. The foreign nucleic acid sequence also encodes at least two flanking sequences which flank at least the RNA guided DNA binding protein nuclease and the one or more guide RNAs. As known to those of skill in the art, flanking sequences are placed at opposite ends of a particular nucleic acid sequence such that the particular nucleic acid sequence is between the flanking sequences. According to one aspect, the flanking sequences include at least a sequence which is identical to a corresponding sequence on a selected chromosome. According to one aspect, such flanking sequences allow a cell to insert the foreign nucleic acid sequence into its genomic DNA at a cut site using well-understood mechanisms such as homologous recombination or nonhomologous end joining.

According to certain aspects, when the foreign nucleic acid sequence is expressed by the germline cell, one or more of an RNA guided DNA binding protein and one or more or a plurality of guide RNAs are produced. The RNA guided DNA binding protein and a guide RNA produces a complex of the RNA guided DNA binding protein, the guide RNA and a double stranded DNA target sequence. In this aspect, the RNA is said to guide the DNA binding protein to the double stranded DNA target sequence for binding thereto. This aspect of the present disclosure may be referred to as co-localization of the RNA and DNA binding protein to or with the double stranded DNA.

DNA binding proteins within the scope of the present disclosure may include those which create a double stranded break (which may be referred to as a DNA binding protein nuclease), those which create a single stranded break (referred to as a DNA binding protein nickase) or those which have no nuclease activity (referred to as a nuclease null DNA binding protein) but otherwise bind to target DNA. In this manner, a DNA binding protein-guide RNA complex may be used to create a double stranded break at a target DNA site, to create a single stranded break at a target DNA site or to localize a transcriptional regulator protein or domain, which may be expressed by the cell, at a target DNA site so as to regulate expression of target DNA. According to certain aspects, the foreign nucleic acid sequence may encode one or more of a DNA binding protein nuclease, a DNA binding protein nickase or a nuclease null DNA binding protein. The foreign nucleic acid sequence may also encode one or more transcriptional regulator proteins or domains or one or more donor nucleic acid sequences that are intended to be inserted into the genomic DNA. According to one aspect, the foreign nucleic acid sequence encoding an RNA guided nuclease-null DNA binding protein further encodes the transcriptional regulator protein or domain fused to the RNA guided nuclease-null DNA binding protein. According to one aspect, the foreign nucleic acid sequence encoding one or more RNAs further encodes a target of an RNA-binding domain and the foreign nucleic acid encoding the transcriptional regulator protein or domain further encodes an RNA-binding domain fused to the transcriptional regulator protein or domain.

Accordingly, expression of a foreign nucleic acid sequence by a germline cell may result in a double stranded break, a single stranded break and/or transcriptional activation or repression of the genomic DNA. Donor DNA may be inserted at the break site by cell mechanisms such as homologous recombination or nonhomologous end joining. It is to be understood that expression of a foreign nucleic acid sequence as described herein may result in a plurality of double stranded breaks or single stranded breaks at various locations along target genomic DNA, including one or more or a plurality of gene sequences, as desired.

Aspects of the present disclosure are directed to using the foreign nucleic acid sequence as a gene drive. The concept of a gene drive is known to those of skill in the art and refers to a foreign nucleic acid sequence which when expressed is capable of inserting itself into the genome of the cell into which it has been introduced. The concept of a gene drive is provided in Windbichler et al., Nature, doi:10.1038/nature09937 (2011) and Burt, Proc. R. Soc. Lond. B (2003) 270, 921-928 (2003) each of which is hereby incorporated by reference in their entireties.

According to one aspect of the present disclosure, the foreign nucleic acid sequences described herein act as gene drives when introduced into a germline cell. In one aspect, the foreign nucleic acid sequence is expressed by the germline cell to produce an RNA guided DNA binding protein and a guide RNA. The guide RNA is complementary to a target DNA sequence on a chromosome. The RNA guided DNA binding protein and the guide RNA co-localize to the target DNA, and the target DNA is cleaved in a site specific manner. The target DNA may be a target DNA site on one or both chromosomes of a chromosome pair. The foreign nucleic acid sequence is then inserted into the genomic DNA at the target DNA cut site, for example, by homologous recombination. The foreign nucleic acid sequence may be inserted into the genomic DNA at one or both chromosomes of a chromosome pair if each chromosome has been cleaved in a site specific manner by the RNA guided DNA binding protein. If inserted into both chromosomes of a chromosome pair, then the germline cell is homozygous for the foreign nucleic acid sequence. In an alternate embodiment, the foreign nucleic acid sequence is inserted into a first chromosome of a chromosome pair. The inserted foreign nucleic acid sequence is then expressed by the cell and the RNA guided DNA binding protein and the guide RNA co-localize at or to a second chromosome of a chromosome pair which is then cleaved in a site specific manner, just as was the first chromosome. The cleaved target DNA in the second chromosome is then repaired, for example by homologous recombination, using the first chromosome as a template. In this manner, the second chromosome is repaired to include the foreign nucleic acid sequence resulting in a germline cell that is homozygous for the foreign nucleic sequence, i.e., the foreign nucleic acid sequence is present in both the first and second chromosome of the chromosome pair. The mechanisms by which cells repair damaged, cleaved or cut genomic DNA are well known. Aspects of the present disclosure take advantage of these cell mechanisms in combination with DNA binding protein nucleases or nickases to create a gene drive with desired foreign genetic material that inserts into the genomic DNA of cells wherein the cell becomes homozygous for the foreign genetic material. The foreign genetic material is then passed on to progeny to create a population of transgenic organisms having one or more desired traits.

Using the concept of a foreign nucleic acid sequence as described herein as a gene drive, methods are provided for incorporating foreign genetic material into a wild population of organisms. Methods are provided for making a cell homozygous for foreign genetic material. Methods are provided for spreading a genetic modification through a wild population of organisms. Methods are provided for spreading a human designed genetic modification through a wild population of organisms. Methods are provided for pan genome engineering by spreading a human designed genetic modification through a wild population of organisms. Methods are provided for editing the genome of a wild species. Methods are provided for editing multiple loci of genomic DNA of an organism. Methods are provided for the multiplexed editing of genomic loci. Methods are provided for reversibly editing a locus or multiple loci of genomic DNA of an organism.

Based upon the desired function of the gene drive described herein, methods are provided for controlling gene flow through a wild population of organisms. Methods are provided for suppressing expansion of a target population of an organism. Methods are provided for decreasing or eliminating a target population of an organism. Methods are providing for increasing a target population of an organism. Methods are provided for reducing or eliminating vector born diseases, such as malaria. Methods are provided for decreasing the spread of disease by a target organism, such as an insect vector population. Methods are provided for disrupting a gene responsible for disease transmission by a target organism. Methods are provided for disrupting a Y chromosome in a germline cell. Methods are provided for disrupting an X chromosome in a germline cell. Methods are provided for controlling invasive pests. Methods are provided for preserving species threatened by ecological change.

According to certain aspects, a method of blocking gene flow from an engineered organism to the wild-type population is provided which includes 1) the insertion of a recoded copy of a gene whose phenotype when disrupted is dominant negative lethal into a distal region of the same chromosome in which the wild-type copy is encoded and 2) the insertion of a selfish genetic element, i.e. RNA guided gene drive as described herein and as the term gene drive is understood by those of skill in the art, that copies itself in place of the wild-type version of the same gene, such that any offspring from an engineered organism and an engineered organism contain two functional copies of the recoded gene, while the offspring of an engineered organism and a wild-type organism will possess only a single copy of the recoded gene after the selfish genetic element replaces the wild-type copy and cannot copy the recoded gene onto the wild-type chromosome.

According to certain aspects, a method of blocking gene flow between a subpopulation bearing a unique sequence and the remaining population is provided which includes 1) releasing a first selfish genetic element that spreads exclusively using the unique sequence and inserts part of a gene whose phenotype when disrupted is dominant negative elsewhere in the genome, 2) releasing a second selfish genetic element that exclusively spreads using the partial gene sequences and inserts a recoded version of the gene and also disrupts the wild-type copy of the gene such that 1) all offspring resulting from matings between an organism bearing the first selfish genetic element and an organism bearing the second selfish genetic element contain the second selfish genetic element in which both wild-type copies of the gene are disrupted but are replaced by recoded copies elsewhere in the genome, and 2) any cross between an organism containing the second selfish genetic element and a wild-type organism generates no progeny because the wild-type copy of the gene is lost and not replaced by a recoded copy.

According to certain aspects, a method of biasing the sex ratio of offspring is provided including using one or more chromosomes that together 1) encode an RNA-guided nuclease that is expressed exclusively during pre-meiosis and 2) express guide RNAs that target the nuclease to cleave sequences uniquely found on one of the sex chromosomes such that viable gametes contain fewer instances of the targeted chromosome than is typical of an unmodified organism.

According to certain aspects, a method of biasing the sex ratio of a population is provided including using a sex chromosome that encodes an RNA-guided nuclease that is expressed exclusively during pre-meiosis and also expresses guide RNAs that target the nuclease to cleave sequences uniquely found on the opposite sex chromosome such that viable gametes predominantly contain the sex chromosome encoding the RNA-guided nuclease.

According to certain aspects, a method of biasing the sex ratio of offspring towards the heterogametic sex (for example XY) is provided including using a chromosome engineered with a copy of an essential gene normally present on the X chromosome and a selfish genetic element that copies itself in place of the wild-type essential gene on the X chromosome such that female progeny are not developmentally viable due to loss of the essential gene while male progeny survive due to the copy on the engineered chromosome.

According to certain aspects, a method of biasing the sex ratio of a population towards the heterogametic sex (for example XY) is provided including using a Y chromosome engineered with a copy of an essential gene normally present on the X chromosome and a selfish genetic element that copies itself in place of the wild-type essential gene on the X chromosome such that female progeny are not developmentally viable due to loss of the essential gene while male progeny survive due to the copy on the engineered Y chromosome.

According to certain aspects, a method of biasing the sex ratio of offspring towards the homogametic sex (for example XX in mammals) including using one or more chromosomes that together 1) encode an RNA-guided nuclease and 2) express guide RNAs that target the nuclease to cleave sequences uniquely found on the heterogametic sex chromosome (for example XY in mammals) such that the heterogametic sex chromosome of any offspring that would normally develop as the heterogametic sex (e.g. males in mammals) is destroyed.

According to certain aspects, a method of biasing the sex ratio of a population towards the homogametic sex (for example XX in mammals) including using a homogametic sex chromosome that 1) encodes an RNA-guided nuclease and 2) expresses guide RNAs that target the nuclease to cleave sequences uniquely found on the heterogametic sex chromosome (for example XY in mammals) such that the heterogametic sex chromosome of any offspring that would normally develop as the heterogametic sex (e.g. males in mammals) is destroyed.

According to certain aspects, a method of biasing the sex ratio of a population towards males includes the release of engineered males that encode a selfish genetic element that copies itself in place of a gene required for female fertility. According to one aspect, the selfish genetic element is encoded on the Y chromosome. According to one aspect, the Y chromosome also encodes a selfish genetic element targeting an essential gene present on the X chromosome.

According to certain aspects, a method of population control is provided including the release into a population to be removed of engineered male organisms containing a Y chromosome that encodes 1) a selfish genetic element that copies itself in place of a gene required for female fertility and 2) a second selfish genetic element that copies itself in place of an essential gene on the X chromosome such that female offspring are nonviable, and the release into a population to be protected of organisms encoding a third selfish genetic element that changes the sequence of the essential gene on the X chromosome such that the offspring of matings between males containing the first and second selfish genetic elements and females containing the third selfish genetic element give rise to fertile male but sterile female offspring.

According to certain aspects, a method is provided of altering a eukaryotic germline cell of an organism including introducing into the germline cell a first foreign nucleic acid sequence encoding an RNA guided DNA binding protein nuclease and one or more guide RNAs, and including corresponding promoter sequences and a first flanking sequence and a second flanking sequence, wherein the one or more guide RNAs are complementary to one or more target locations on genomic DNA of a first chromosome and a second chromosome of a chromosome pair of the germline cell, wherein the nucleic acid sequence encoding the RNA guided DNA binding protein nuclease and the nucleic acid sequence encoding the one or more guide RNAs are between the first flanking sequence and the second flanking sequence,

wherein the first flanking sequence includes a first sequence identical to a first portion of the target location on the first chromosome or the second chromosome of the genomic DNA, wherein the second flanking sequence includes a second sequence identical to a second portion of the target location on the first chromosome or the second chromosome of the genomic DNA, wherein at least one copy of the sequence located between the first flanking sequence and the second flanking sequence that is cut and replaced by the foreign nucleic acid sequence is required for the organism to survive or to produce viable offspring, expressing the first foreign nucleic acid sequence to produce the RNA guided DNA binding protein nuclease and the one or more RNAs wherein the RNA guided DNA binding protein nuclease and an associated guide RNA co-localize to an associated target location on the first chromosome of the genomic DNA and the second chromosome of the genomic DNA and the RNA guided DNA binding protein nuclease cleaves the first chromosome of the genomic DNA at the target location in a cleavage site specific manner and cleaves the second chromosome of the genomic DNA at the target location in a cleavage site specific manner,

inserting the first foreign nucleic acid sequence into the first chromosome of the chromosome pair of the genomic DNA at the cleavage site, and inserting the first foreign nucleic acid sequence into the second chromosome of the chromosome pair of the genomic DNA at the cleavage site to render the germline cell homozygous for the foreign nucleic acid sequence, and performing the above expression and insertion steps at developmental stage at which the sequence located between the first flanking sequence and the second flanking sequence that is cut and replaced by the foreign nucleic acid sequence to produce a genetic load is no longer required for that organism to survive or produce fertile offspring.

According to certain aspects, a method for targeted population suppression or extinction according to such methods described herein includes releasing an RNA-guided genetic load drive into the targeted population.

A genetic load as that term is used herein may refer to the difference between the fitness of the theoretically optimal genotype in a population and the fitness of the observed average genotype in a population. The term genetic load may also refer to a reduction in the mean fitness for a population compared to the maximum fitness.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1A is a schematic of a standard RNA-guided Cas9 gene drive showing expression of Cas9 and guide RNA, co-localization, cutting of a target gene to create a break and insertion of the RNA guided Cas9 gene drive into the target gene at the cut site by homologous recombination. FIG. 1B depicts a chromosomal view of gene drive inheritance. FIG. 1C depicts a mutation preventing the co-localization of Cas9 and a guide RNA so as to prevent cutting of the target DNA and insertion of the gene drive. FIG. 1D depicts the use of a plurality of guide RNAs and cut sites to avoid a mutation from preventing insertion of the gene drive. FIG. 1E depicts NHEJ deleting all recognition sites to create a drive resistant allele. FIG. 1F depicts NHEJ creating a drive resistant allele that deleteriously disrupts an essential gene. (SEQ ID NO:1-3)

FIG. 2A depicts the use of a single drive to edit multiple distal sites by including appropriate guide RNAs. FIG. 2B depicts the use of subsequent drives to update changes made by a first drive. FIG. 2C depicts the use of a restorative drive to all changes back to the wild-type, leaving only the Cas9 gene and guide RNAs.

FIG. 3 depicts the use of two gene drives that target the same essential gene using different guide RNAs that become incompatible when crossed due to the loss of both copies. Releasing this type of drive into a population will artificially split it into as many species as there are drives.

FIG. 4A depicts the use of meiotic gene drives to eliminate the competing sex chromosome during meiosis, producing a biased pool of gametes. Drive expression is limited to pre-meiosis to prevent the drive from being lethal. FIG. 4B depicts that meiotic sex chromosome silencing can be circumvented using two orthogonal Cas9 nucleases. Cas9 A causes the cassette encoding Cas9 B to be copied from the sex chromosome to an autosome early in development, where it is free to eliminate the competing sex chromosome during meiosis. FIG. 4C depicts that zygotic X-drives can target the Y chromosome for destruction in the fertilized egg, yielding exclusively female progeny. FIG. 4D depicts that zygotic Y-drives can encode an essential gene normally found on the X chromosome and utilize a gene drive cassette to eliminate that gene from the X. The drive copies itself from the paternal to the maternal X, leaving female zygotes without any copies of the essential gene.

FIG. 5A depicts population suppression using dual-function Y-drives where the A Y-drive that also contains a sterile-daughter capability (Y-Drive-SD) acts as a zygotic drive in the wild-type population, which can lead to extinction. FIG. 5B depicts that upon encountering a standard drive that recodes the essential gene, the Y-Drive-SD will no longer drive. The sterile-daughter effect will prevent mutual drive invasion until the invasive population collapses.

FIG. 6A depicts a method for controlling low- and high-mobility invasive species where Y-drive male Asian carp released throughout invaded habitats such as the Mississippi can be used to eradicate Asian carp populations. A protective recoding drive can be available to protect Asian populations in the event of deliberate human transport of Y-drive males. FIG. 6B depicts releasing Y-Drive-SD male rats into invasive rat populations which will initiate local eradication while a standard drive protectively recodes the native rat populations of Eurasia. The Y-Drive-SD construct will eradicate invasive rats from most islands and many continental areas. Stowaway-mediated gene flow will be limited by the sterile-daughter effect of Y-Drive-SD in recoded populations. The process can be repeated with new drives once recoded stowaways successfully invade rat-free habitats.

FIG. 7 depicts drive-mediated regulating of endogenous genes where RNA-guided transcriptional regulators can modulate the activity of distant genes without editing their sequence. Regulation will be evolutionarily stable during the lifetime of the drive if the drive nuclease requires the regulator for proper expression.

FIG. 8 depicts inducing speciation from an unmodified wild-type population. Two successive gene drives targeting a subpopulation bearing a unique allele (depicted as a unique gene) can induce genetic incompatibility with the remaining wild-type population. The first drive spreads using the unique allele and recodes a gene that displays a dominant lethal phenotype when disrupted. The second drive spreads using the recoded gene and eliminates the first drive. Any mating with a wild-type organism causes the second drive to disrupt the wild-type copy, causing lethality in all progeny.

FIG. 9 depicts a method described herein wherein Drive 1 recodes a male essential gene on the Y chromosome (if present) and a recessive essential gene on the X chromosome while inserting itself (i.e., being inserted by a cell mechanism such as homologous recombination after expression of Cas9 and guide RNA and localization of the Cas9 and guide RNA to a target nucleic acid sequence) adjacent to the unique gene. Drive 2 spreads through the modified X chromosome, eliminates Drive 1 from the autosome, and also jumps into the modified Y chromosome if present. Upon mating with a wild-type organism, Drive 2 eliminates the essential genes from the X or Y chromosomes. The only viable progeny are hybrid females that retain a single recoded copy of the recessive essential gene on the X chromosome. These can mate freely with males that have been modified with Drive 2, permitting wild-type genes to spread into the engineered population. Mating with a wild-type male yields only more hybrid females, preventing gene flow back into the wild-type population.

FIG. 10 depicts a method for making a zygotic Y-drive as described herein.

FIG. 11A depicts one method of making a sterile-daughter Y chromosome that eliminates a gene required for female fertility. Y chromosome (Y-SD) is not a true drive, but incorporates a limited gene drive mechanism that replaces a gene essential for female fertility with the drive. FIG. 11B depicts that in the daughters of Y-SD males, the drive similar replaces the maternal copy of the female fertility gene, causing sterility. If no gene specific for female fertility is known, a fertility gene required for both sexes can be target and complemented with an extra copy elsewhere on the Y chromosome.

FIG. 12A depicts a method of making a Y-drive that includes sterile-daughter capability (Y-drive-SD) (Y-drive+sterile-daughter Y chromosomes. Upon mating with a wild-type female, Y-drive-SD replaces or disrupts both an essential gene and a female fertility gene. Males survive due to complementation from the extra copy on Y-drive-SD. As depicted in FIG. 12B, in female progeny, the drives similarly replace the maternal copies, leaving daughters embryonically nonviable. Y-drive-SD consequently exhibits drive. As depicted in FIG. 12C, females modified with a protective drive have the sites flanking the essential gene recoded. Y-Drive-SD can only replace the fertility gene. As depicted in FIG. 12D, female progeny are still viable, but lack any copies of the fertility gene and are consequently infertile. Because males do not gain any fitness benefit from the absence of sisters, Y-drive-SD will not drive in a protected population. However, its only fitness cost is that incurred by the drive itself, so it will be removed from the population only slowly. The loss of female reproductive output will result in potent population suppression. Because of this, protected individuals will find it difficult to invade populations where Y-drive-SD is common. Similarly, Y-Drive-SD will not be able to invade protected populations.

FIG. 13A is a schematic showing that biased inheritance of ADE2 is readily visible in S. cerevisiae. Mutations in ADE2 generate a red phenotype on adenine-limiting media due to the buildup of red pigments. Mating a red mutant haploid to a wild-type haploid produce cream-colored diploids, which yield 50% red and 50% cream-colored progeny upon sporulation. Biased inheritance of ADE2 is readily visible in S. cerevisiae. FIG. 13B is a schematic showing that when haploids with a gene drive targeting ADE2 mate with wild-type haploids in the presence of Cas9, cutting and subsequent replacement or disruption of ADE2 will lead to red diploids that yield exclusively red progeny. FIG. 13C is an image of S. cerevisiae colonies. Diploids produced by mating wild-type and ADE2::sgRNA gene drive haploids yield cream-colored colonies in the absence of Cas9 or when the target site is removed by recoding but uniformly red colonies when both are present, demonstrating Cas9-dependent disruption of the wild-type ADE2 copy. FIG. 13D are images of spores from 15 dissected tetrads that produce uniformly red colonies on adenine-limited plates, confirming disruption of the ADE2 gene inherited from the wild-type parent. In the absence of the target site or Cas9, normal 2:2 segregation is observed.

FIG. 14A is a schematic showing that the ADE2-targeting gene drive was modified to carry URA3 as a cargo gene. FIG. 14 B is an image of diploids produced by mating wild-type URA3⁻ haploid yeast with haploids encoding the gene drive carrying URA3 which were sporulated and tetrads dissected to isolate colonies arising from individual spores. All of these grew when replica-plated onto plates lacking uracil, demonstrating that the drive successfully copied URA3 in all diploids. FIG. 14C is a schematic showing that the ABD1-targeting gene drive cuts and recodes the tail end of the essential ABD1 gene. Gene drives and cargo genes remain intact upon copying and can spread by targeting both non-essential and essential genes.

FIG. 15A is a schematic of a phylogenomic tree indicating the relationships between wild-type strains selected for gene drive testing. Adapted with permission from Macmillan Publishers Ltd: Nature 458:337-341, copyright 2009. FIG. 15 B depicts quantitative PCR results of the extent of inheritance-biasing in diverse yeast strains depicting the relative abundance of wild-type and drive-containing alleles in diploids arising from matings between SK1 haploids bearing gene drives and diverse wild-type haploid strains. “No Cas9” and “No Target” refer to the haploid cells containing the ADE2 drive element mated to wild-type haploids in the absence of Cas9 or to a wild-type strain with a mutation in the targeted sequence that prevents cutting. “2nd gen” refers to the haploid progeny of an earlier mating.

FIG. 16 depicts inheritance of a split RNA-guided gene drive element in a population encoding the required non-driving element.

FIG. 17 depicts inheritance of a split RNA-guided gene drive element in a population lacking the required non-driving element as compared to inheritance of a normal gene.

FIG. 18 depicts inheritance of dependent or daisy chain gene drives.

FIG. 19 depicts various dependent gene drive architectures.

FIG. 20 is directed to the importance of initial release size for daisy chain gene drives and shows a graph of incidence of A, B, and C alleles following the release of an A-B-C daisy chain gene drive at either 5% or 0.5% of the wild-type population. A, B, C each impose a dominant fitness cost of 0.02.

FIG. 21 is directed to consequences of repeatedly releasing daisy chain gene drives and shows a graph of incidence of A, B, and C alleles following the release of an A-B-C daisy chain gene drive at either 0.02% of the wild-type population once (top) or each generation (bottom). A, B, C each impose a dominant fitness cost of 0.1. Note the scale difference in the Y-axis.

### DETAILED DESCRIPTION

Embodiments of the present disclosure are based on the use of RNA guided DNA binding proteins to co-localize with guide RNA at a target DNA site and act as gene drives. Such DNA binding proteins are readily known to those of skill in the art to bind to DNA for various purposes. Such DNA binding proteins may be naturally occurring. DNA binding proteins included within the scope of the present disclosure include those which may be guided by RNA, referred to herein as guide RNA. According to one aspect, the guide RNA is between about 10 to about 500 nucleotides. According to one aspect, the RNA is between about 20 to about 100 nucleotides. According to this aspect, the guide RNA and the RNA guided DNA binding protein form a co-localization complex at the DNA.

Embodiments of the present disclosure are directed to designing genome editing systems using a Cas/guide RNA system such that separate portions or components of the Cas/guide RNA system for a given target locus, such as the Cas component, the guide RNA component and/or the payload or cargo gene component, can be separately encoded as foreign nucleic acid sequences within the genome of a cell, such as a germline cell, and that one or more separately encoded portions or components of the Cas/guide RNA system is dependent upon an initiator molecule or molecules for ability to perform as a gene drive. According to one aspect, the initiator molecule or molecules which promote the foreign nucleic acid sequence being a gene drive are not included within the foreign nucleic acid sequence. The foreign nucleic acid is thereby dependent on the separately encoded initiator molecule or molecules to function as a gene drive.

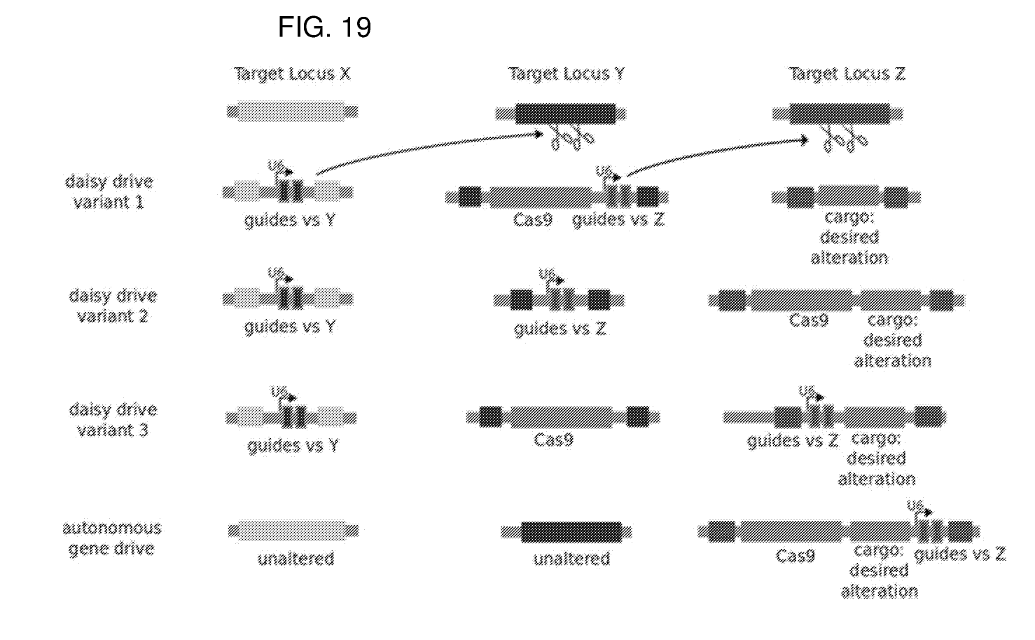

According to one aspect and with respect to FIG. 19, a cell is modified to be homozygous for a given desired gene at a target locus so as to exhibit a desired phenotype. A first nucleic acid sequence encoding one or more guide RNAs complementary to a target locus Y (guide RNA for locus Y) is inserted into the genome of a cell, such as a germline cell at a target locus X. This first nucleic acid sequence is incapable of functioning as a gene drive. A second nucleic acid sequence encoding Cas9 or Cas9 and one or more guide RNAs complementary to a target locus Z (guide RNA for locus Z) or one or more guide RNAs complementary to a target locus Z (guide RNA for locus Z) is inserted into the genome of the cell at a target locus Y. A third nucleic acid sequence encoding a desired gene or a desired gene and Cas9 or a desired gene and one or more guide RNAs complementary to target locus Z is inserted into the germline cell at a target locus Z. In this manner, various constructs and systems are possible where one construct expresses the guide RNA which facilitates a different construct to be a gene drive. For example, the first nucleic acid sequence expresses the guide RNA for locus Y which facilitates the second nucleic acid sequence being a gene drive wherein the guide RNA for locus Y cuts locus Y when Cas 9 is present such as by a result of being expressed by a different or the same construct and the second nucleic acid sequence is inserted into the target locus Y, such as by homologous recombination. For example, the second nucleic acid sequence expresses the guide RNA for locus Z which facilitates the third nucleic acid sequence being a gene drive wherein the guide RNA for locus Z cuts locus Z when Cas 9 is present such as by a result of being expressed by a different or the same construct and the third nucleic acid sequence encoding the desired gene is inserted into the target locus Z, such as by homologous recombination. As an alternative, the third nucleic acid sequence includes the guide RNA for locus Z which cuts locus Z when Cas 9 is present such as by a result of being expressed by a different construct and the third nucleic acid sequence encoding the desired gene is inserted into the target locus Z, such as by homologous recombination.

One of skill will readily envision where only two constructs are used, such as where a first construct encodes Cas9 which is not capable of being a gene drive and a second construct which encodes the guide RNA for locus Z and a desired gene. In the presence of Cas9, the desired gene is inserted into the target locus Z, such as by homologous recombination.

One of skill will also readily envision that a chain of separate constructs can be used where the ability of any particular construct to function as a gene drive is determined by the presence of the corresponding guide RNA in a separate or different construct. In this manner, a second construct is dependent on a first construct for its ability to function as a gene drive insofar as the first construct encodes the guide RNA corresponding to the second construct. Also, an Nth construct is dependent on an Nth-1 construct for its ability to function as a gene drive insofar as the Nth-1 construct encodes the guide RNA corresponding to the Nth construct.

DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

Bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February, 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June, 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February, 2008) hereby incorporatd by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June, 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January, 2011) each of which are hereby incorporated by reference in their entireties.

Exemplary DNA binding proteins having nuclease activity function to nick or cut double stranded DNA. Such nuclease activity may result from the DNA binding protein having one or more polypeptide sequences exhibiting nuclease activity. Such exemplary DNA binding proteins may have two separate nuclease domains with each domain responsible for cutting or nicking a particular strand of the double stranded DNA. Exemplary polypeptide sequences having nuclease activity known to those of skill in the art include the McrA-HNH nuclease related domain and the RuvC-like nuclease domain. Accordingly, exemplary DNA binding proteins are those that in nature contain one or more of the McrA-HNH nuclease related domain and the RuvC-like nuclease domain.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinke et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is shown below. See Deltcheva et al., Nature 471, 602-607 (2011) hereby incorporated by reference in its entirety.

(SEQ ID NO: 4)
MDKKYSIGLDIGTNSVGWAVITDEYKVPSKKFKVLGNTDRHSIKKNLIGA
LLFDSGETAE
ATRLKRTARRRYTRRKNRICYLQEIFSNEMAKVDDSFFHRLEESFLVEED
KKHERHPIFG
NIVDEVAYHEKYPTIYHLRKKLVDSTDKADLRLIYLALAHMIKFRGHFLI
EGDLNPDNSD
VDKLFIQLVQTYNQLFEENPINASGVDAKAILSARLSKSRRLENLIAQLP
GEKKNGLFGN
LIALSLGLTPNFKSNFDLAEDAKLQLSKDTYDDDLDNLLAQIGDQYADLF
LAAKNLSDAT
LLSDILRVNTEITKAPLSASMIKRYDEHHQDLTLLKALVRQQLPEKYKEI
FFDQSKNGYA
GYIDGGASQEEFYKFIKPILEKMDGTEELLVKLNREDLLRKQRTFDNGSI
PHQIHLGELH
AILRRQEDFYPFLKDNREKIEKILTFRIPYYVGPLARGNSRFAWMTRKSE
ETITPWNFEE
VVDKGASAQSFIERMTNFDKNLPNEKVLPKHSLLYEYFTVYNELTKVKYV
TEGMRKPAFL
SGEQKKAIVDLLFKTNRKVTVKQLKEDYFKKIECFDSVEISGVEDRFNAS
LGTYHDLLKI
IKDKDFLDNEENEDILEDIVLTLTLFEDREMIEERLKTYAHLFDDKVMKQ
LKRRRYTGWG
RLSRKLINGIRDKQSGKTILDFLKSDGFANRNFMQLIHDDSLTFKEDIQK
AQVSGQGDSL
HEHIANLAGSPAIKKGILQTVKVVDELVKVMGRHKPENIVIEMARENQTT
QKGQKNSRER
MKRIEEGIKELGSQILKEHPVENTQLQNEKLYLYYLQNGRDMYVDQELDI
NRLSDYDVDH
IVPQSFLKDDSIDNKVLTRSDKNRGKSDNVPSEEVVKKMKNYWRQLLNAK
LITQRKFDNL
TKAERGGLSELDKAGFIKRQLVETRQITKHVAQILDSRMNTKYDENDKLI
REVKVITLKS
KLVSDFRKDFQFYKVREINNYHHAHDAYLNAVVGTALIKKYPKLESEFVY
GDYKVYDVRK
MIAKSEQEIGKATAKYFFYSNIMNFFKTEITLANGEIRKRPLIETNGETG
EIVWDKGRDF
ATVRKVLSMPQVNIVKKTEVQTGGFSKESILPKRNSDKLIARKKDWDPKK
YGGFDSPTVA
YSVLVVAKVEKGKSKKLKSVKELLGITIMERSSFEKNPIDFLEAKGYKEV
KKDLIIKLPK
YSLFELENGRKRMLASAGELQKGNELALPSKYVNFLYLASHYEKLKGSPE
DNEQKQLFVE
QHKHYLDEIIEQISEFSKRVILADANLDKVLSAYNKHRDKPIREQAENII
HLFTLTNLGA
PAAFKYFDTTIDRKRYTSTKEVLDATLIHQSITGLYETRIDLSQLGGD

According to one aspect, the specificity of gRNA-directed Cas9 cleavage is used as a mechanism for genome engineering and as a drive gene. According to one aspect, hybridization of the gRNA need not be 100 percent in order for the enzyme to recognize the gRNA/DNA hybrid and affect cleavage. Some off-target activity could occur. For example, the S. pyogenes system tolerates mismatches in the first 6 bases out of the 20 bp mature spacer sequence in vitro. According to one aspect, greater stringency may be beneficial in vivo when potential off-target sites matching (last 14 bp) NGG exist within the human reference genome for the gRNAs.

According to certain aspects, specificity may be improved. When interference is sensitive to the melting temperature of the gRNA-DNA hybrid, AT-rich target sequences may have fewer off-target sites. Carefully choosing target sites to avoid pseudo-sites with at least 14 bp matching sequences elsewhere in the genome may improve specificity. The use of a Cas9 variant requiring a longer PAM sequence may reduce the frequency of off-target sites. Directed evolution may improve Cas9 specificity to a level sufficient to completely preclude off-target activity, ideally requiring a perfect 20 bp gRNA match with a minimal PAM. Accordingly, modification to the Cas9 protein is a representative embodiment of the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase.

An exemplary DNA binding protein is an RNA guided DNA binding protein nuclease of a Type II CRISPR System, such as a Cas9 protein or modified Cas9 or homolog of Cs9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null Cas9 protein.

According to certain aspects of methods of RNA-guided genome regulation described herein, Cas9 is altered to reduce, substantially reduce or eliminate nuclease activity. According to one aspect, Cas9 nuclease activity is reduced, substantially reduced or eliminated by altering the RuvC nuclease domain or the HNH nuclease domain. According to one aspect, the RuvC nuclease domain is inactivated. According to one aspect, the HNH nuclease domain is inactivated. According to one aspect, the RuvC nuclease domain and the HNH nuclease domain are inactivated. According to an additional aspect, Cas9 proteins are provided where the RuvC nuclease domain and the HNH nuclease domain are inactivated. According to an additional aspect, nuclease-null Cas9 proteins are provided insofar as the RuvC nuclease domain and the HNH nuclease domain are inactivated. According to an additional aspect, a Cas9 nickase is provided where either the RuvC nuclease domain or the HNH nuclease domain is inactivated, thereby leaving the remaining nuclease domain active for nuclease activity. In this manner, only one strand of the double stranded DNA is cut or nicked.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinke et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 or Cas9N and exhibits reduced or eliminated nuclease activity, or nuclease activity is absent or substantially absent within levels of detection. According to this aspect, nuclease activity for a Cas9N may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. pyogenes and protein sequences having at least 30%, 40%, 50%, 60%, 70%, 80%, 90%, 95%, 98% or 99% homology thereto and being a DNA binding protein, such as an RNA guided DNA binding protein.

According to one aspect, an engineered Cas9-gRNA system is provided which enables RNA-guided genome regulation in cells by tethering transcriptional activation domains to either a nuclease-null Cas9 or to guide RNAs. According to one aspect of the present disclosure, one or more transcriptional regulatory proteins or domains (such terms are used interchangeably) are joined or otherwise connected to a nuclease-deficient Cas9 or one or more guide RNA (gRNA). The transcriptional regulatory domains correspond to targeted loci. Accordingly, aspects of the present disclosure include methods and materials for localizing transcriptional regulatory domains to targeted loci by fusing, connecting or joining such domains to either Cas9N or to the gRNA. According to certain aspects, methods are provided for regulating endogenous genes using Cas9N, one or more gRNAs and a transcriptional regulatory protein or domain. According to one aspect, an endogenous gene can be any desired gene, referred to herein as a target gene.

According to one aspect, a Cas9N-fusion protein capable of transcriptional activation is provided. According to one aspect, a VP64 activation domain (see Zhang et al., Nature Biotechnology 29, 149-153 (2011) hereby incorporated by reference in its entirety) is joined, fused, connected or otherwise tethered to the C terminus of Cas9N. According to one method, the transcriptional regulatory domain is provided to the site of target genomic DNA by the Cas9N protein. According to one method, a Cas9N fused to a transcriptional regulatory domain is provided within a cell along with one or more guide RNAs. The Cas9N with the transcriptional regulatory domain fused thereto bind at or near target genomic DNA. The one or more guide RNAs bind at or near target genomic DNA. The transcriptional regulatory domain regulates expression of the target gene. According to a specific aspect, a Cas9N-VP64 fusion activated transcription of reporter constructs when combined with gRNAs targeting sequences near the promoter, thereby displaying RNA-guided transcriptional activation.

According to one aspect, a gRNA-fusion protein capable of transcriptional activation is provided. According to one aspect, a VP64 activation domain is joined, fused, connected or otherwise tethered to the gRNA. According to one method, the transcriptional regulatory domain is provided to the site of target genomic DNA by the gRNA. According to one method, a gRNA fused to a transcriptional regulatory domain is provided within a cell along with a Cas9N protein. The Cas9N binds at or near target genomic DNA. The one or more guide RNAs with the transcriptional regulatory protein or domain fused thereto bind at or near target genomic DNA. The transcriptional regulatory domain regulates expression of the target gene. According to a specific aspect, a Cas9N protein and a gRNA fused with a transcriptional regulatory domain activated transcription of reporter constructs, thereby displaying RNA-guided transcriptional activation.

According to one aspect, the transcriptional regulator protein or domain is a transcriptional activator. According to one aspect, the transcriptional regulator protein or domain upregulates expression of the target nucleic acid. According to one aspect, the transcriptional regulator protein or domain is a transcriptional repressor. According to one aspect, the transcriptional regulator protein or domain downregulates expression of the target nucleic acid. Transcriptional activators and transcriptional repressors can be readily identified by one of skill in the art based on the present disclosure.

According to one aspect, the foreign nucleic acid sequence encodes two or more guide RNAs with each RNA being complementary to an adjacent site in the DNA target nucleic acid and also encodes at least one RNA guided DNA binding protein nickase and being guided by the two or more RNAs, wherein the two or more RNAs and the at least one RNA guided DNA binding protein nickase are expressed and wherein the at least one RNA guided DNA binding protein nickase co-localizes with the two or more RNAs to the DNA target nucleic acid and nicks the DNA target nucleic acid resulting in two or more adjacent nicks. According to certain aspects, the two or more adjacent nicks are on the same strand of the double stranded DNA. According to one aspect, the two or more adjacent nicks are on the same strand of the double stranded DNA and result in homologous recombination. According to one aspect, the two or more adjacent nicks are on different strands of the double stranded DNA. According to one aspect, the two or more adjacent nicks are on different strands of the double stranded DNA and create double stranded breaks. According to one aspect, the two or more adjacent nicks are on different strands of the double stranded DNA and create double stranded breaks resulting in nonhomologous end joining. According to one aspect, the two or more adjacent nicks are on different strands of the double stranded DNA and are offset with respect to one another. According to one aspect, the two or more adjacent nicks are on different strands of the double stranded DNA and are offset with respect to one another and create double stranded breaks. According to one aspect, the two or more adjacent nicks are on different strands of the double stranded DNA and are offset with respect to one another and create double stranded breaks resulting in nonhomologous end joining. According to one aspect, the two or more adjacent nicks are on different strands of the double stranded DNA and create double stranded breaks resulting in fragmentation of the target nucleic acid thereby preventing expression of the target nucleic acid.

According to certain aspects, binding specificity of the RNA guided DNA binding protein may be increased according to methods described herein. According to one aspect, off-set nicks are used in methods of genome-editing. A large majority of nicks seldom result in NHEJ events, (see Certo et al., Nature Methods 8, 671-676 (2011) hereby incorporated by reference in its entirety) thus minimizing the effects of off-target nicking. In contrast, inducing off-set nicks to generate double stranded breaks (DSBs) is highly effective at inducing gene disruption. According to certain aspects, 5′ overhangs generate more significant NHEJ events as opposed to 3′ overhangs. Similarly, 3′ overhangs favor HR over NHEJ events, although the total number of HR events is significantly lower than when a 5′ overhang is generated. Accordingly, methods are provided for using nicks for homologous recombination and off-set nicks for generating double stranded breaks to minimize the effects of off-target Cas9-gRNA activity.

Germline cells according to the present disclosure include any germline cell into which foreign nucleic acids can be introduced and expressed as described herein. It is to be understood that the basic concepts of the present disclosure described herein are not limited by cell type. Germline cells according to the present disclosure include eukaryotic germline cells, prokaryotic germline cells, animal germline cells, mammalian germline cells, plant germline cells, insect germline cells, fungal germline cells, archael germline cells, eubacterial germline cells and the like. Further, germline cells include any in which it would be beneficial or desirable to introduce a foreign nucleic acid sequence described herein.

Target nucleic acids include any nucleic acid sequence to which a co-localization complex as described herein can be useful to either cut, nick or regulate. Target nucleic acids include genes. For purposes of the present disclosure, DNA, such as double stranded DNA, can include the target nucleic acid and a co-localization complex can bind to or otherwise co-localize with the DNA at or adjacent or near the target nucleic acid and in a manner in which the co-localization complex may have a desired effect on the target nucleic acid. Such target nucleic acids can include endogenous (or naturally occurring) nucleic acids and exogenous (or foreign) nucleic acids. One of skill based on the present disclosure will readily be able to identify or design guide RNAs and Cas9 proteins which co-localize to a DNA including a target nucleic acid. One of skill will further be able to identify transcriptional regulator proteins or domains which likewise co-localize to a DNA including a target nucleic acid. DNA includes genomic DNA, mitochondrial DNA, viral DNA or exogenous DNA.

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Transcriptional regulator proteins or domains which are transcriptional activators or transcriptional repressors may be readily identifiable by those skilled in the art based on the present disclosure and the particular germline cell.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Cas9 Gene Drives

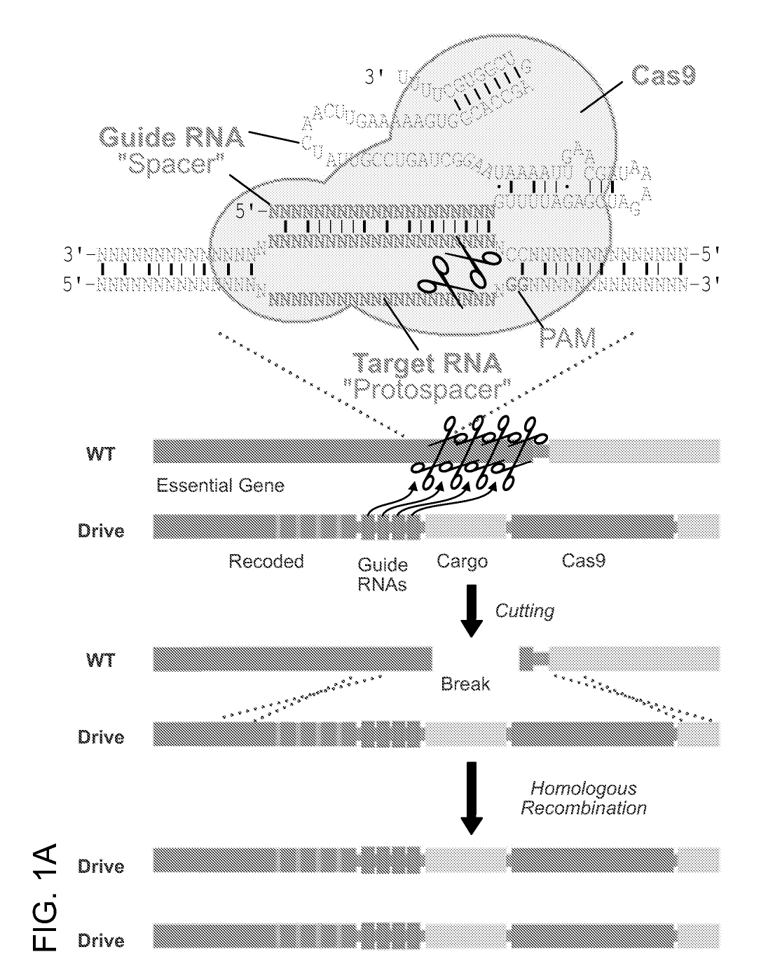

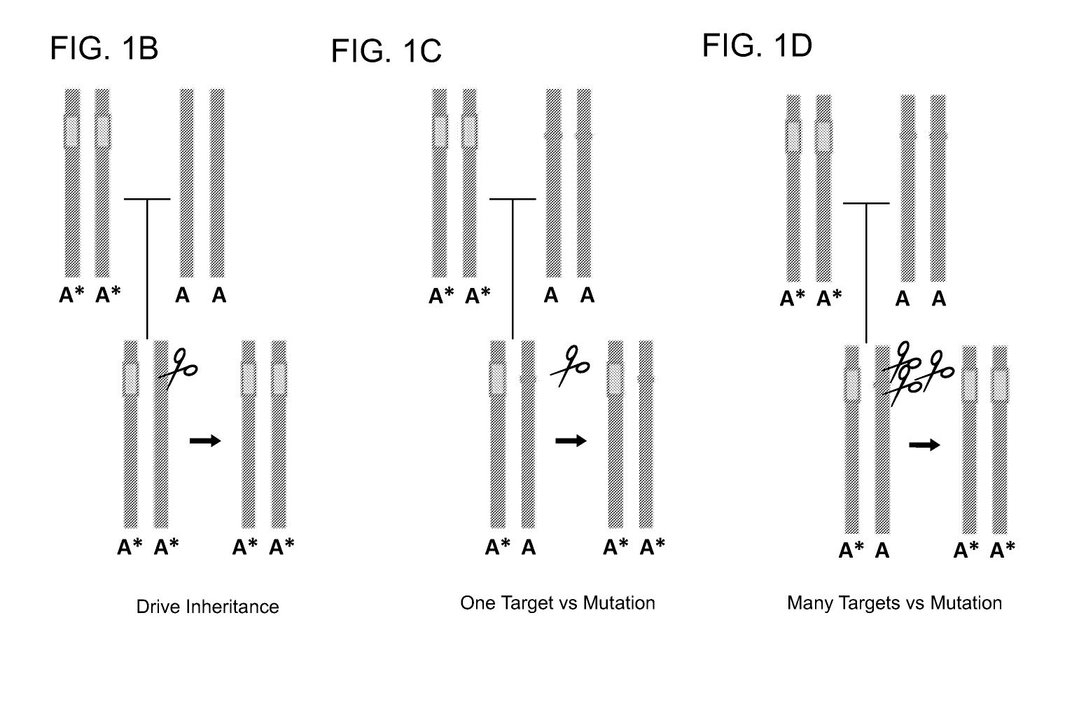

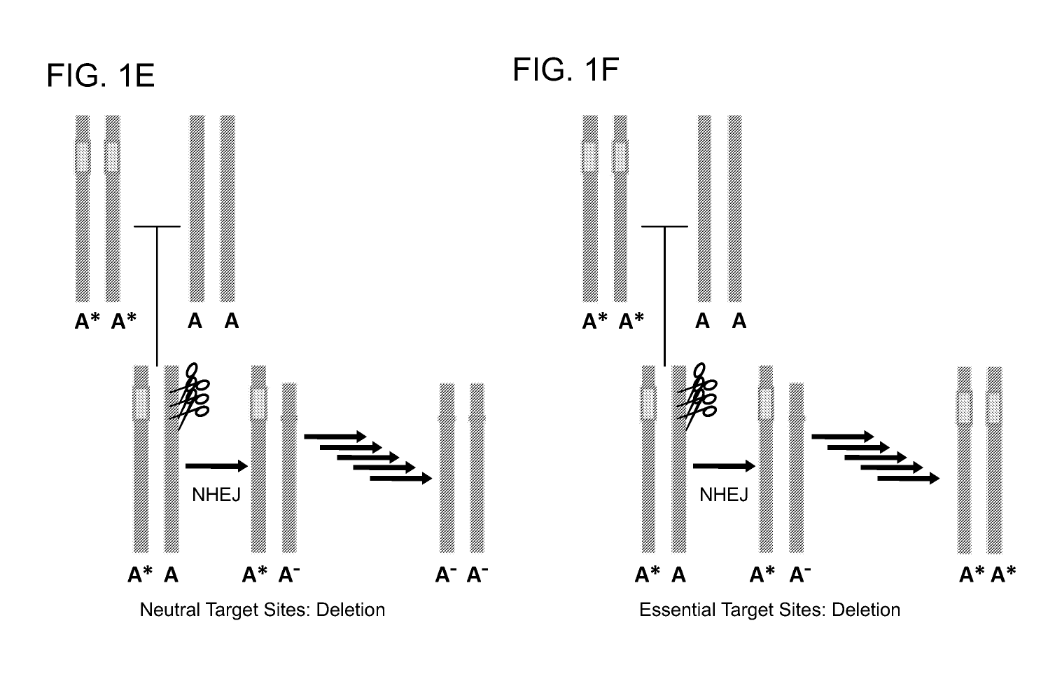

FIG. 1A is a schematic showing the colocalization of a guide RNA and a Cas9 nuclease to a target DNA. The target DNA sites include a “protospacer” sequence that matches the “spacer” of the guide RNA and a short protospacer-adjacent motif (PAM) required for Cas9 binding. An exemplary Cas9 gene drive shown in FIG. 1A is a foreign nucleic acid sequence as described herein (which may be referred to as a “cassette” as that term is understood by those of skill in the art) that encodes at least an RNA guided Cas9 nuclease, one or more guide RNAs that are complementary to one or more target DNA sites, such as within an adjacent essential gene, and flanking sequences. The cassette may also include any cargo genetic material, such as one or more genes to be expressed by the germline cell into which the cargo genetic material is to be inserted or to be expressed by the descendants of that cell. The Cas9 gene drive is expressed to produce Cas9 and the one or more guide RNAs which then co-localize at one or more target DNA sites on a first chromosome of a chromosome pair and where the Cas9 then cleaves the target DNA on the first chromosome in a site specific manner producing a double stranded break. Homology-directed repair of Cas9-induced double-strand breaks then inserts the Cas9 gene drive into the first chromosome at the cut site. According to certain aspects, the inserted Cas9 gene drive is then expressed to produce Cas9 and the one or more guide RNAs which then co-localize at the one or more target DNA sites on the second chromosome of the chromosome pair and then cleaves the target DNA in a site specific manner producing a double stranded break. Homology directed repair of the Cas9-induced double stranded breaks uses the intact Cas9 drive-containing chromosome as a template thereby inserting the Cas9 drive into the second chromosome of the chromosome pair to create a chromosome pair that is homozygous for the Cas9 gene drive. This is depicted in FIG. 1B.

FIG. 1C depicts that a mutation at the target DNA site may prevent cutting by a single Cas9 protein. Accordingly, aspects of the present disclosure depicted in FIG. 1D provide multiple guide RNAs for multiple target DNA sites. In this manner, RNA-guided gene drives can overcome resistant alleles by targeting multiple adjacent sequences such that no single mutation can confer resistance. The chances that a target site will not be cleaved due to a mutation are greatly reduced as the target DNA site will likely be cleaved in multiple locations thereby providing a cut site into which the RNA guided gene drive can be inserted. As shown in FIG. 1E, repair using the alternative non-homologous end-joining (NHEJ) pathway can delete all recognition sites, creating a drive-resistant allele that outcompetes the drive. FIG. 1F depicts that NHEJ events that disrupt an essential gene are highly deleterious and will be selected against in favor of the drive. Choosing target sites within an essential gene ensures that incorrect repair events cannot create resistant alleles by deleting all of the targeted sequences. Non-homologous end joining can create small insertions or deletions at the junction of the repair which can prevent binding of a guide RNA and Cas9 to the repair site. As a result, the RNA guided gene drive may not be able to be inserted into this site on the chromosome if there is only a single guide RNA for this site encoded in the RNA guided gene drive. However, having the RNA guided gene drive encode for multiple guide RNAs targeting multiple sites and therefore making multiple cut sites will increase the odds that the RNA guided gene drive will be inserted into the chromosome. Further, aspects of the present disclosure are directed to targeting multiple sites for Cas9 within an essential gene as a method for improving efficiency of inserting the RNA guided gene drive into a chromosome.

### Example II

### Editing and Regulating Multiple Endogenous Loci

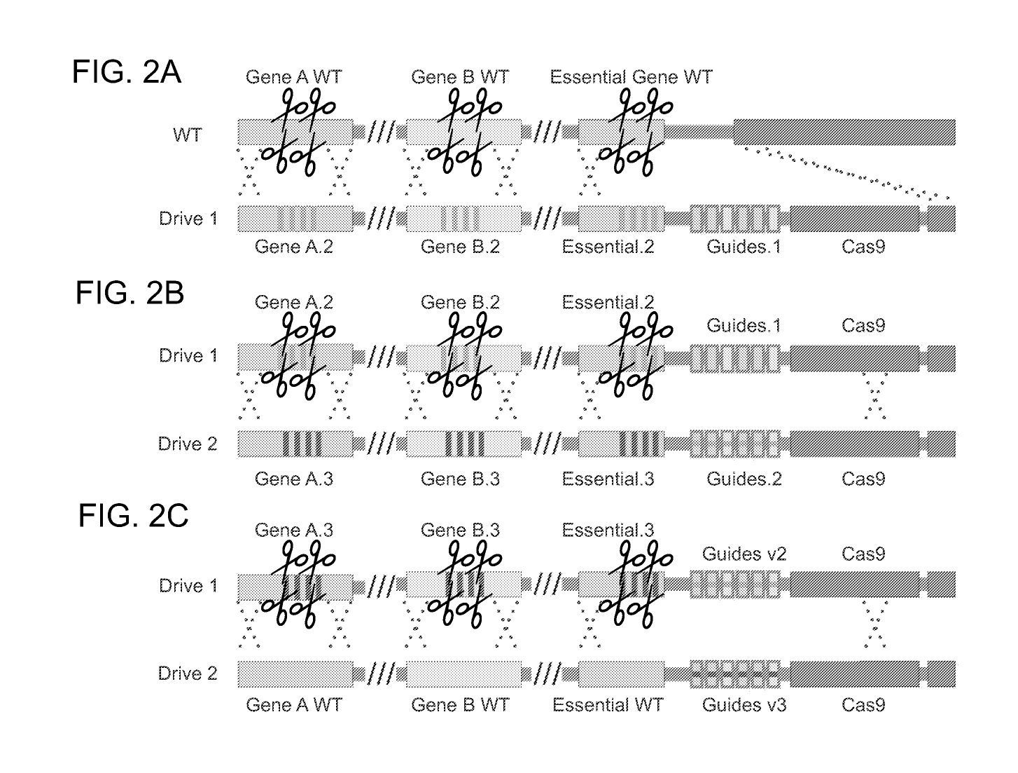

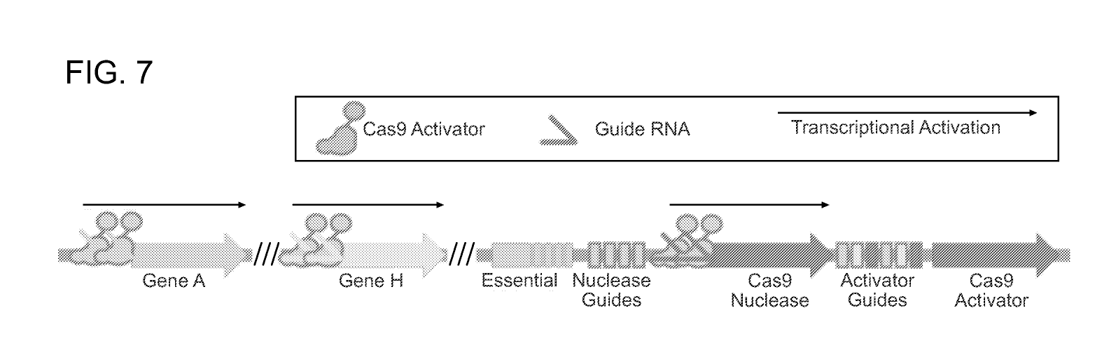

According to certain aspects of the present disclosure, an RNA-guided gene drive can edit multiple endogenous alleles distal to the drive itself by incorporating guide RNAs that target the respective wild-type alleles. As shown in FIG. 2A, multiple guide RNAs are provided for a plurality of gene sequences, i.e., Gene A WT, Gene B WT and Essential Gene WT. Expression of the multiple guide RNAs and the Cas9 protein results in each of the genes being modified by Cas9. According to one aspect, the Cas9 drive may also include one or more orthogonal nuclease null Cas9 proteins and associated guide RNAs with transcriptional regulators which can colocalize to a target DNA site so that the transcriptional regulator can regulate the gene. See FIG. 7. As shown in FIG. 2B, additional subsequent Cas9 drives can be used to update the changes that have been previously made by a prior Cas9 drive. For example, if a particular drive causes unexpected side effects, a second drive released as an update can overwrite one or all modifications encoded by the first drive. According to this aspect, the second drive can be designed to remove the existing inserted drive and then itself be inserted into the chromosome. In addition, one or more genes that have been modified by a prior RNA guided gene drive may be removed and replaced with a desired gene. Similarly, as depicted in FIG. 2C, a third restorative drive can restore the wild type sequence in all genomic locations except for the presence of the Cas9 gene and guide RNAs required to spread the restorative drive. In this manner, a population can be modified to revert back to its wild type. Accordingly, aspects of the present disclosure are directed to the modification or replacing of a previously inserted RNA guided Cas9 gene drive by introducing a second RNA guided Cas9 gene drive into a previously modified cell using the methods described herein. The second gene drive is designed to make further modifications beyond those made by the first gene drive or to eliminate the modifications made by the first gene drive.

### Example III

### Controlling Spread

According to certain aspects, the spread of RNA-guided gene drives can be limited to a single target species or even a subpopulation by targeting a unique gene or sequence polymorphism associated with the single target species or subpopulation. Because the drive can only cut the unique sequence, it does not spread through non-target populations. Accordingly, aspects of the present disclosure are directed to methods of designing and using RNA guided gene drives that are specific for a unique gene sequence or sequence polymorphism. In this manner one or more guide RNA are designed to be complementary to the unique gene sequence or sequence polymorphism. In this manner, the DNA binding protein is restricted to localizing at the unique gene sequence or sequence polymorphism.

### Example IV

### Inducing Artificial Speciation

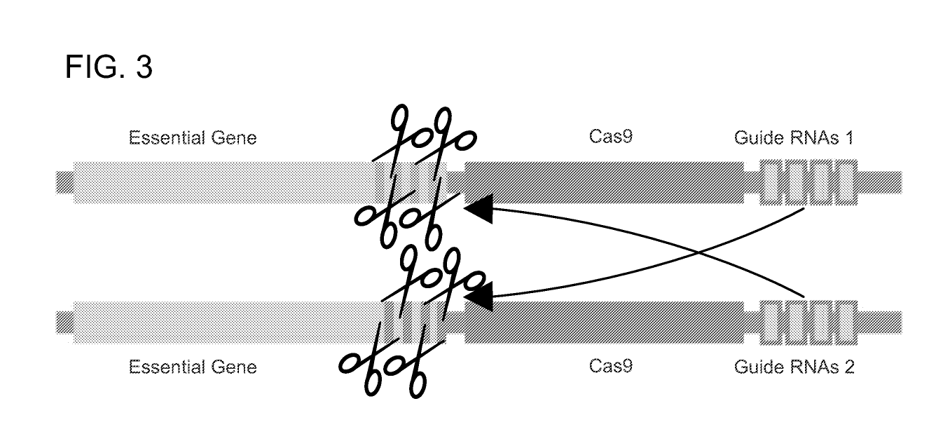

According to certain aspects, methods are provided of using RNA-guided gene drives to block gene flow by causing genetic incompatibility between two populations. According to this aspect and as shown in FIG. 3, two gene drives that target different sequences within the same essential gene using different guide RNAs are released into two populations. The two populations become incompatible because of the loss of each copy of the essential gene within each population. Releasing this type of drive into a population will artificially split it into as many species as there are drives. As shown in exemplary FIG. 3, drive A cuts sequences 1, 2, 3, and 4 within essential gene X. As drive A spreads, it replaces gene X with a version that has sequences 1, 2, 3, and 4 recoded so they can not be cut. Drive B cuts sequences 5, 6, 7, and 8 within gene X, and its version of gene X has these sequences recoded so they can not be cut. Crossing a male with drive A and a female with drive B results in progeny that inherit one chromosome with drive A and gene X with 1/2/3/4 recoded, and one chromosome with drive B and gene X with 5/6/7/8 recoded. Drive A cuts the second copy of gene X at sites 1/2/3/4, because the second copy doesn't have those sites recoded—it only has 5/6/7/8 recoded. Similarly, drive B cuts the first copy of gene X at sites 5/6/7/8 because the first copy doesn't have those sites recoded, it only has 1/2/3/4 recoded. The organism ends up with both copies of gene X cut. Since gene X is essential and the organism lacks an intact copy of gene X to fix them with, the organism dies. Drive A and drive B are accordingly incompatible.

According to this aspect, each drive encodes a different version of the essential gene—a first foreign nucleic acid sequence and a second foreign nucleic acid sequence—that each preserve the amino acid sequence of the essential protein encoded by that gene, but which have different sets of target locations recoded—the first drive has sites 1/2/3/4 recoded to be noncomplementary to guides 1/2/3/4, whereas the second drive has sites 5/6/7/8 recoded to be noncomplementary to guides 5/6/7/8. When the drives are crossed, the first drive cleaves the second drive's copy of the essential gene at 1/2/3/4 because it doesn't have those sites recoded, while the second drive cleaves the first drive's copy of the essential gene at 5/6/7/8 because it doesn't have those sites recoded.

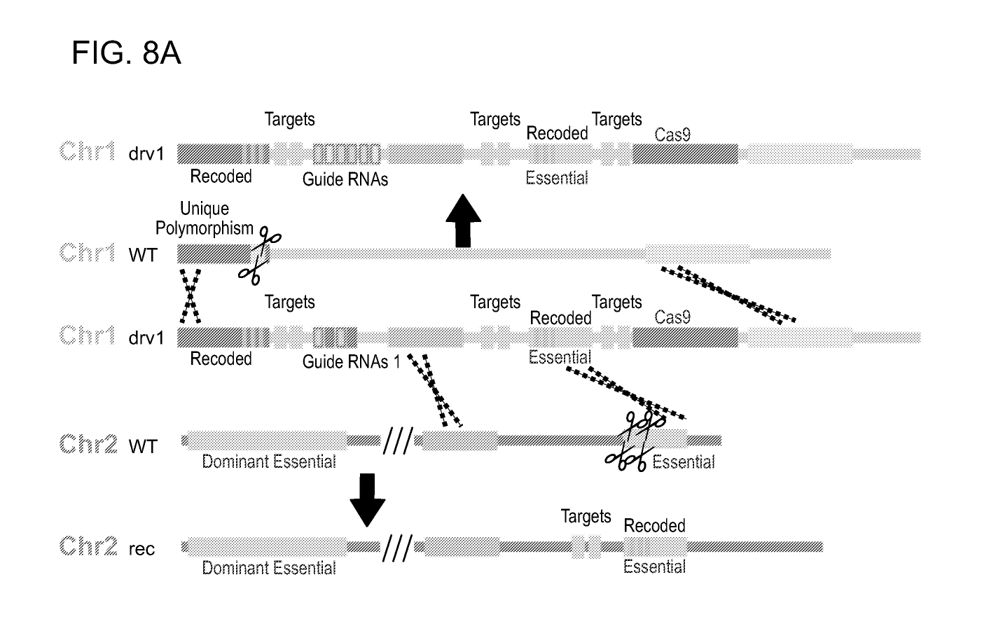

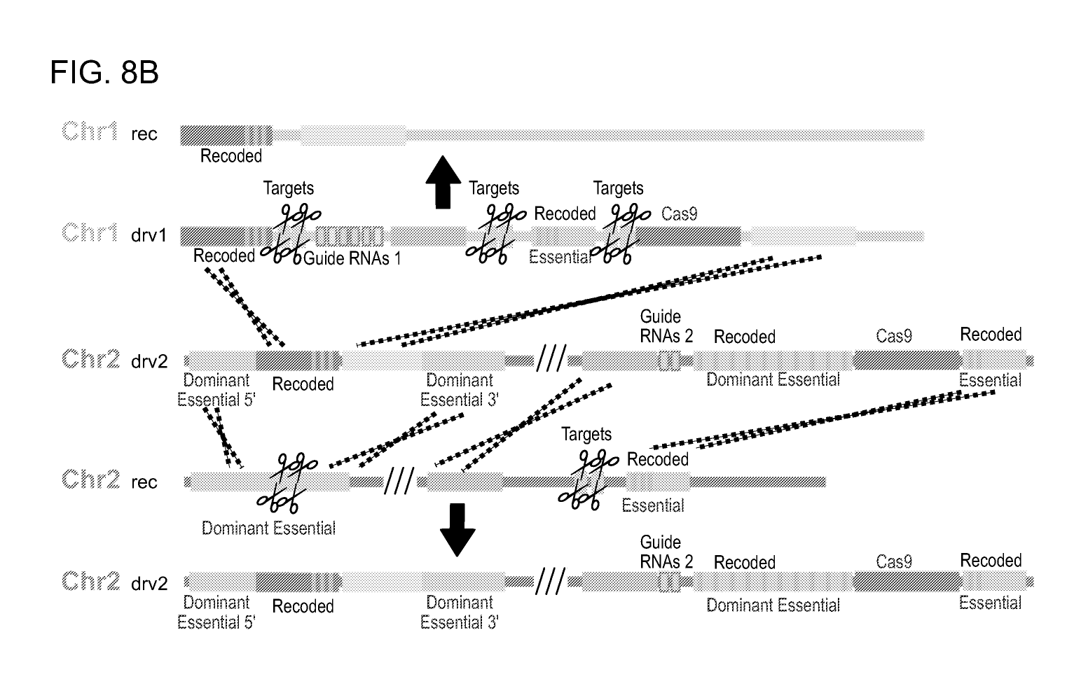

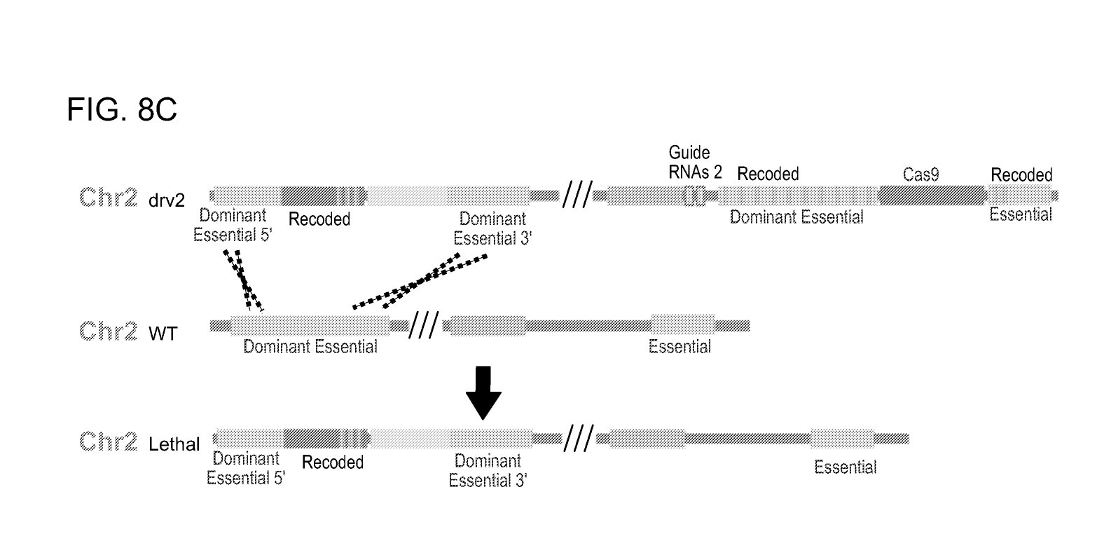

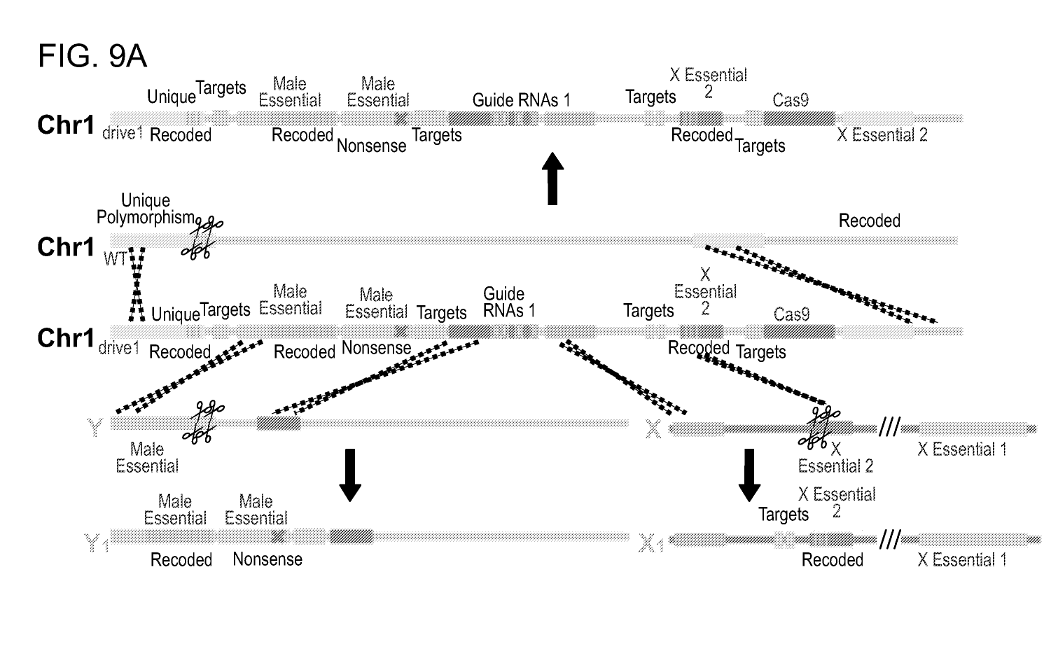

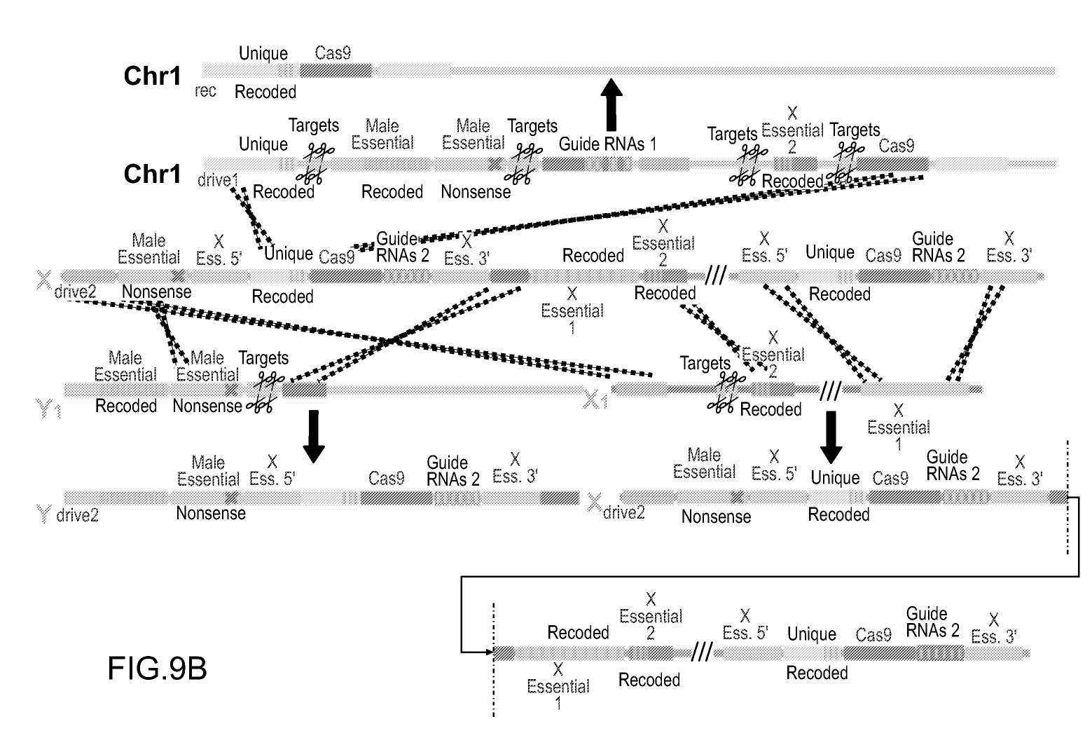

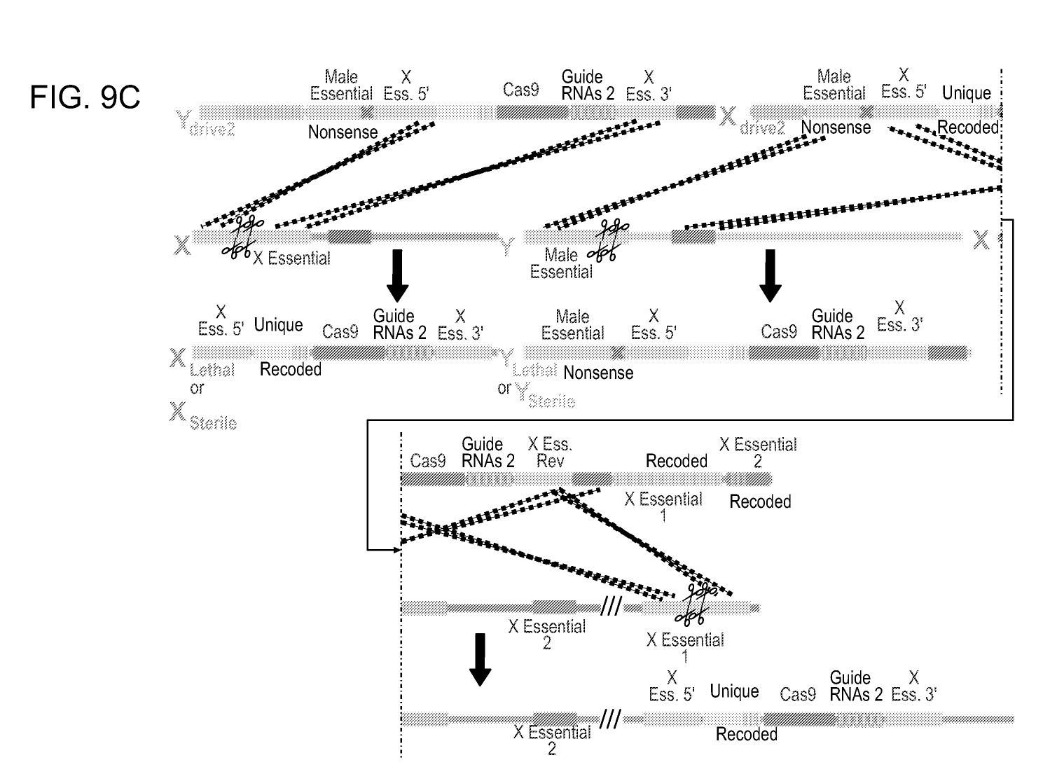

More complex gene drives can block gene flow between an engineered subpopulation and the wild-type population without modifying the latter (see FIG. 8). This barrier can even be one-way, allowing the engineered population to receive but not donate genetic material to the wild-type population (see FIG. 9). These or related designs might be used to prevent other gene drives or genetic modifications from spreading to unmodified members of the same or related species. According to this aspect, only one of the two populations are modified and two separate RNA guided gene drives are used. The first drive creates an insertion site for the second drive. The second drive 1) includes a copy of an essential gene, such that it copies that gene as it spreads into the insertion site created by the first drive, and 2) kills the wild-type copy of that essential gene. When an organism with the second drive mates with an organism with the first drive, the second drive is copied into the insertion site created by the first drive, adding a copy of the essential gene and removing the wild-type copy. Net change in essential genes is 0, so the cell is viable. When an organism with the second drive is mated with a wild-type organism, the drive and its essential gene is unable to be inserted into the wild-type chromosome because it lacks an insertion site. However, the second drive still kills the wild-type copy. Net change in essential genes is −1, so the cell dies. Accordingly, any matings between organisms containing the second drive and wild-type organisms produce no progeny. This aspect of the disclosure provides a method of making transgenic organisms incompatible with the wild-type population to prevent the spread of transgenes.

### Example V

### Population Suppression and Extinction

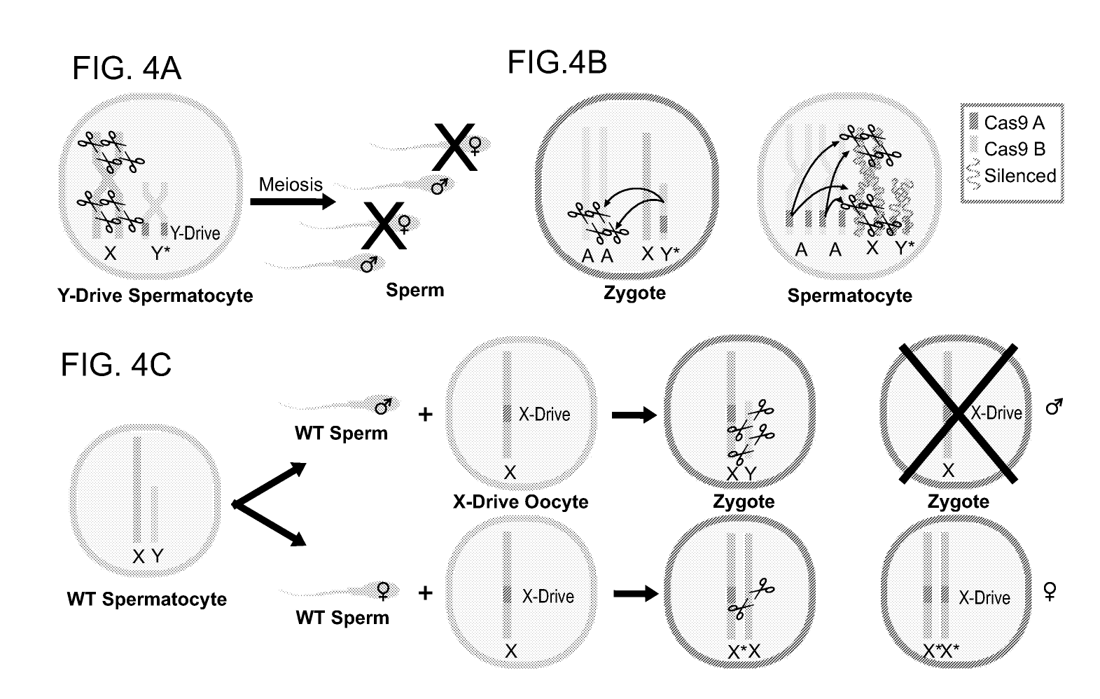

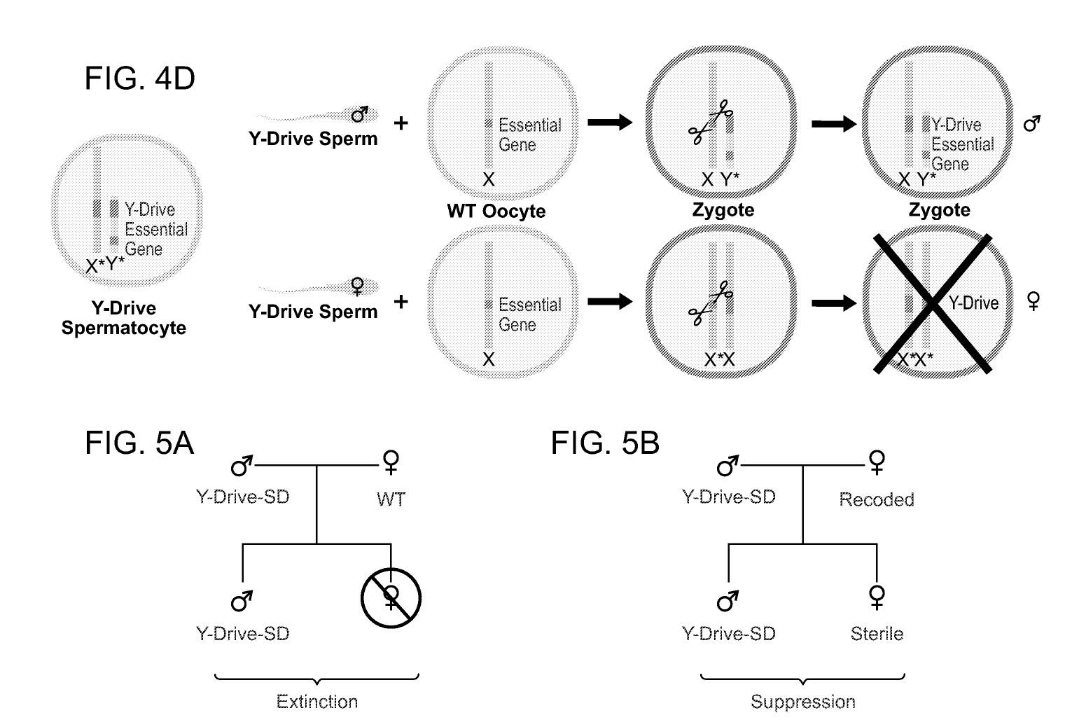

According to certain aspects, RNA-guided nucleases can create several different forms of sex-biasing gene drives that may be useful for population control. As shown in FIG. 4A, expressing Cas9 and guide RNAs targeting the X chromosome exclusively during male meiosis will result in a classical “meiotic” Y-drive analogous to those found in nature. The meiotic gene drive eliminates the competing sex chromosome during meiosis, producing a biased pool of gametes. Drive expression must be strictly limited to pre-meiosis or the drive will be lethal. This type of drive can bias the gametes of organisms employing XY, ZW, and to a lesser extent XO sex-determination systems in favor of either sex. Further with respect to FIG. 4A, an RNA guided gene drive is introduced into the Y chromosome of a spermatocyte. The RNA guided gene drive includes a plurality of guide RNA complementary to target sequences on the X chromosome. When the RNA guided gene drive is expressed, the guide RNAs and the Cas9 nuclease colocalize at the X chromosome where the Cas9 nuclease creates double stranded breaks rendering the X chromosome inoperable. After meiosis, only sperm having the Y chromosome will be viable.

Importantly, orthogonal Cas9 nucleases should be capable of circumventing meiotic chromosome silencing. Aspects of the present disclosure are directed to the use of a first RNA guided gene drive as described herein on a Y chromosome to express Cas9 with suitable guide RNA prior to generation of sperm where the expressed Cas9 and the suitable guide RNA cuts an autosome and a second RNA guided gene drive encoding a second Cas9 and suitable guide RNA is inserted into the autosome. During spermatogenesis, the second RNA guided gene drive in the autosome is expressed and the expressed second Cas9 and suitable guide RNA cuts the X chromosome thereby rendering it inoperable. This method allows the cutting of the X chromosome even in species in which the X and Y chromosomes are not expressed during spermatogenesis. As shown in FIG. 4B, Cas9 causes the cassette encoding Cas9B to be copied from the sex chromosome to an autosome early in development, where it is free to eliminate the competing sex chromosome during meiosis. According to one aspect, RNA guided gene drives that bias for a particular sex are provided on the sex chromosome which they bias the population towards. For example, males have Y chromosomes in mammals, so a drive that biases populations towards males has to be on the Y chromosome, as it then assists the Y chromosome make more copies of itself. As mammalian sex chromosomes are not expressed during meiosis (sperm and egg formation), two copies of Cas9 can be used to facilitate proper insertion of the drives into a chromosome which when expressed will produce a Cas9 that will cut the sex chromosome. The first Cas9 acts early in development, well before sperm formation, and it cuts an autosome (non sex chromosomes that are not silenced during meiosis) thereby allowing insertion of an RNA guided gene drive encoding a second Cas9 into the autosome. This second Cas9 is expressed from the autosome during sperm formation and cuts the X chromosome, thereby ensuring that most viable sperm contain the Y chromosome. Thy above described method therefore uses two RNA guided gene drives where one is inserted into an expressable autosome to bias populations towards males in the situation where sex chromosomes can't express genes during meiosis.

As shown in FIG. 4C, “zygotic” sex-biasing drives eliminate opposite-sex siblings during the zygotic or post-zygotic stage (see Rice and Friberg 2008 hereby incorporated by reference in its entirety). Importantly, they do not require highly specific expression patterns in order to function. X-drives eliminate male zygotes by simply targeting sites on the Y chromosome (FIG. 4C). In Y-drives, a gene drive cassette copies itself in place of an essential gene on the X chromosome, the loss of which is complemented in males by a copy inserted elsewhere on the driving Y chromosome (FIG. 4D). It may be necessary to adjust expression of the transplanted essential gene to account for dosage compensation in some species.

With respect to FIG. 4C, a wild type spermatocyte creates a wild type sperm with an X chromosome and a wild type sperm with a Y chromosome. An oocyte is provided with an RNA guided gene drive with one or more guide RNAs complementary to the Y chromosome, which is referred to as an “X drive oocyte”. When the wild type sperm with the Y chromosome is combined with the oocyte, the RNA guided gene drive is expressed, the guide RNA and the Cas9 nuclease colocalize to the Y chromosome where the Cas9 nuclease creates double stranded breaks in the Y chromosome rendering the Y chromosome inoperable and thereby producing an inoperable zygote. In contrast, when the wild type sperm with the X chromosome is combined with the oocyte, the RNA guided gene drive is expressed, guide RNA complementary to the X chromosome and the Cas9 nuclease colocalize to the X chromosome where the Cas9 nuclease creates double stranded breaks in the X chromosome which is repaired by homologous recombination thereby inserting the RNA guided gene drive into the X chromosome rendering the zygote homozygous for the X chromosome. This copy of the X chromosome remains viable because the double strand breaks are repaired when the RNA guided gene drive is copied. The resulting progeny are all female, because the males are not viable due to loss of the Y chromosome. These females have an advantage over other females that do not encode an RNA guided gene drive because they do not have to compete with male siblings for resources. Consequently, the X chromosome with an RNA guided gene drive has a fitness advantage. With respect to FIG. 4D, zygotic Y-drives encode an essential gene normally found on the X chromosome and utilize a gene drive cassette to eliminate that essential gene from the X chromosome. The Y drive is inserted from the paternal to the maternal X chromosome, leaving female zygotes without any copies of the essential gene. In particular, a spermatocyte is provided with a Y chromosome having an essential gene from the X chromosome and an RNA guided gene drive encoding one or more guide RNAs complementary to the essential gene on the X chromosome. The X chromosome of the spermatocyte also includes the same RNA guided gene drive. The spermatocyte produces a sperm with the Y chromosome and a sperm with the X chromosome. The sperm with the Y chromosome is combined with a wild type oocyte having an X chromosome with the essential gene. When the RNA guided gene drive is expressed, the one or more guide RNAs colocalize with the Cas9 nuclease to the X chromosome where the Cas9 nuclease cuts out the essential gene from the X chromosome. However, since the essential gene from the X chromosome is present on the Y chromosome, the zygote will be viable. In contrast, when the sperm with the X chromosome including the RNA guided gene drive combines with the wild type oocyte with the X chromosome, the expressed one or more guide RNAs colocalize with the Cas9 nuclease to the X chromosome where the Cas9 nuclease cuts out the essential gene from the X chromosome. Since the essential gene no longer exists in the oocyte, the oocyte is not viable.

The fitness benefit of zygotic drives will vary depending on the degree of sibling competition, the extent of parental investment in offspring, and the mating dynamics in adults. All zygotic Y-drives should produce at least as many if not more sons than competing wild-type Y chromosomes, and none of those sons will have to compete with sisters. Unlike other drive types, the host range of zygotic Y-drives can be restricted by ensuring that matings with specific at-risk related species or non-targeted subpopulations are sterile. This can be accomplished by incorporating guide RNAs that cleave unique sequences on the X-chromosomes to eliminate hybrid males.

Releasing a Y-drive will render the local population extinct if mutations capable of blocking the drive do not quickly arise on the X chromosome or one of the autosomes. The drive can be halted and eventually eliminated by releasing organisms bearing sex chromosomes that are immune to cutting. Close monitoring and control can prevent total extinction.

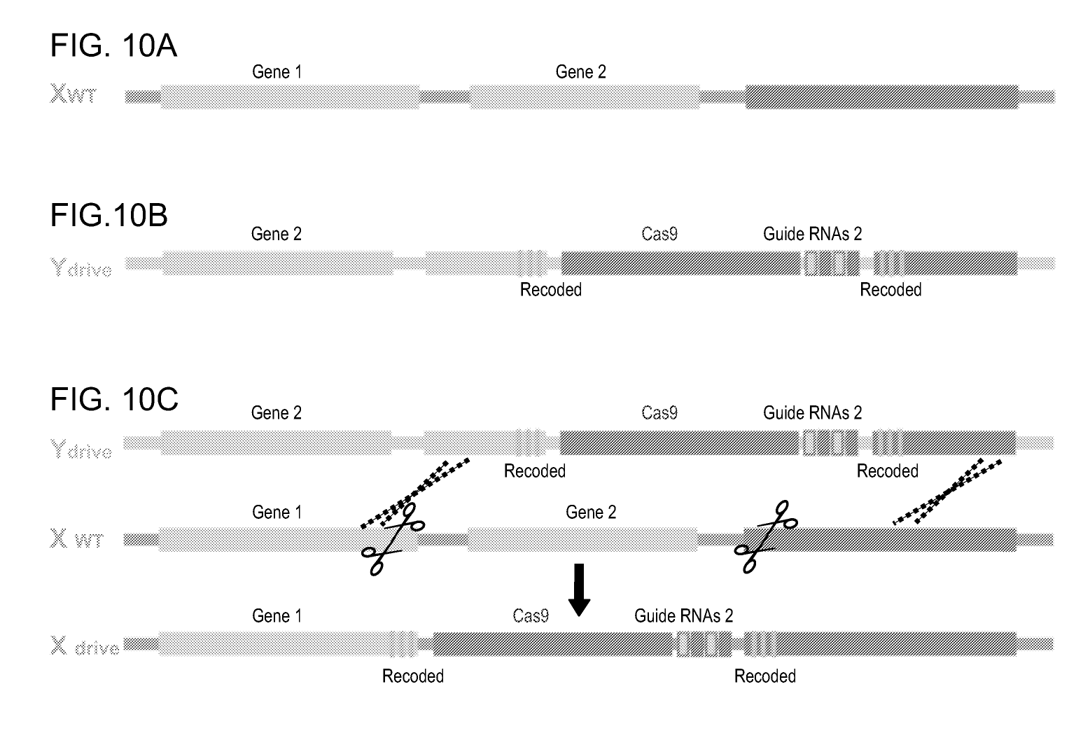

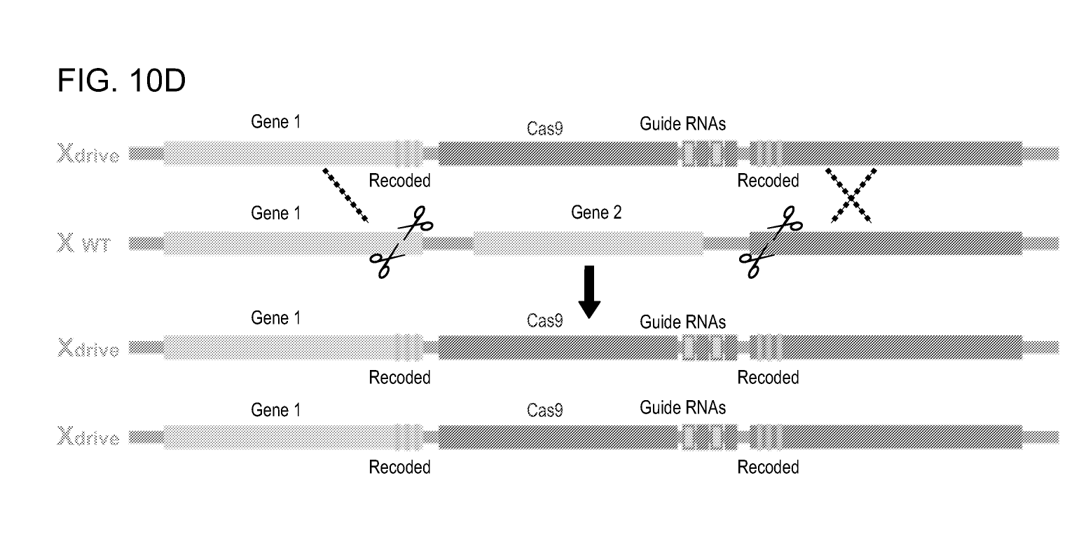

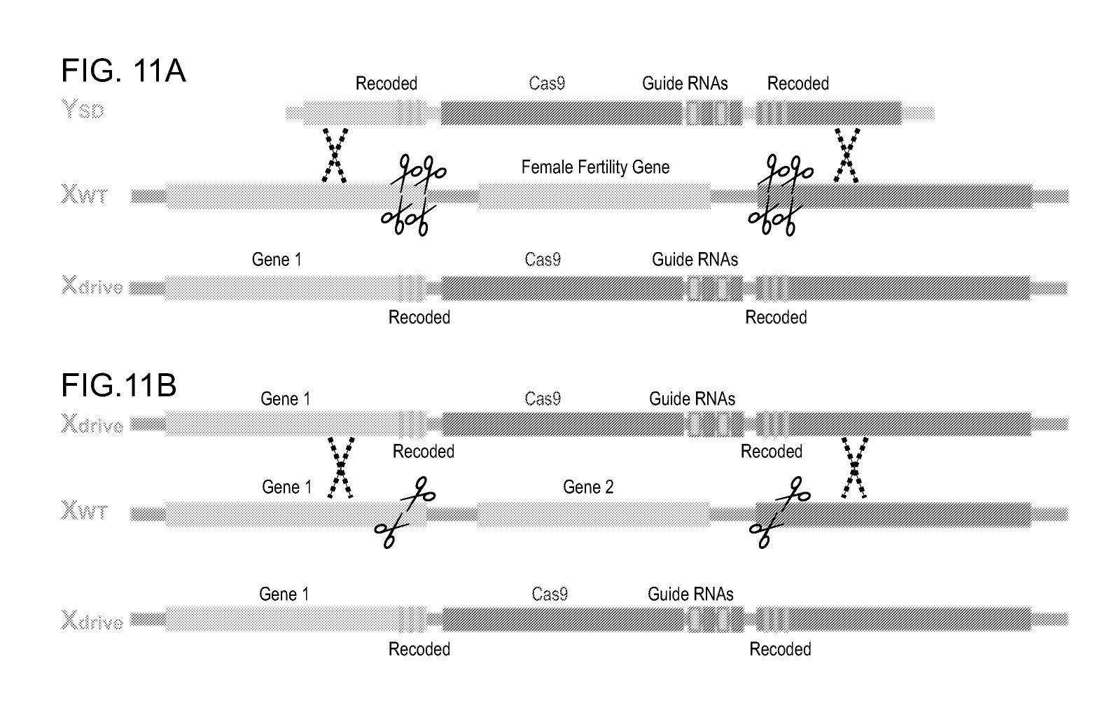

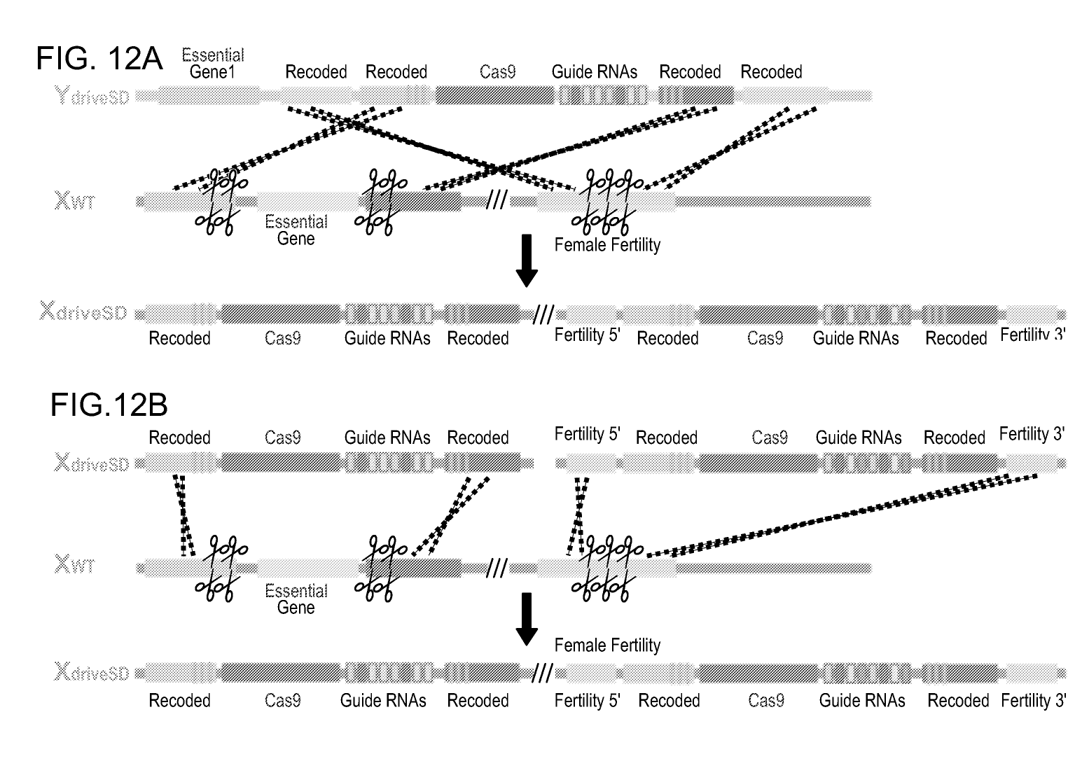

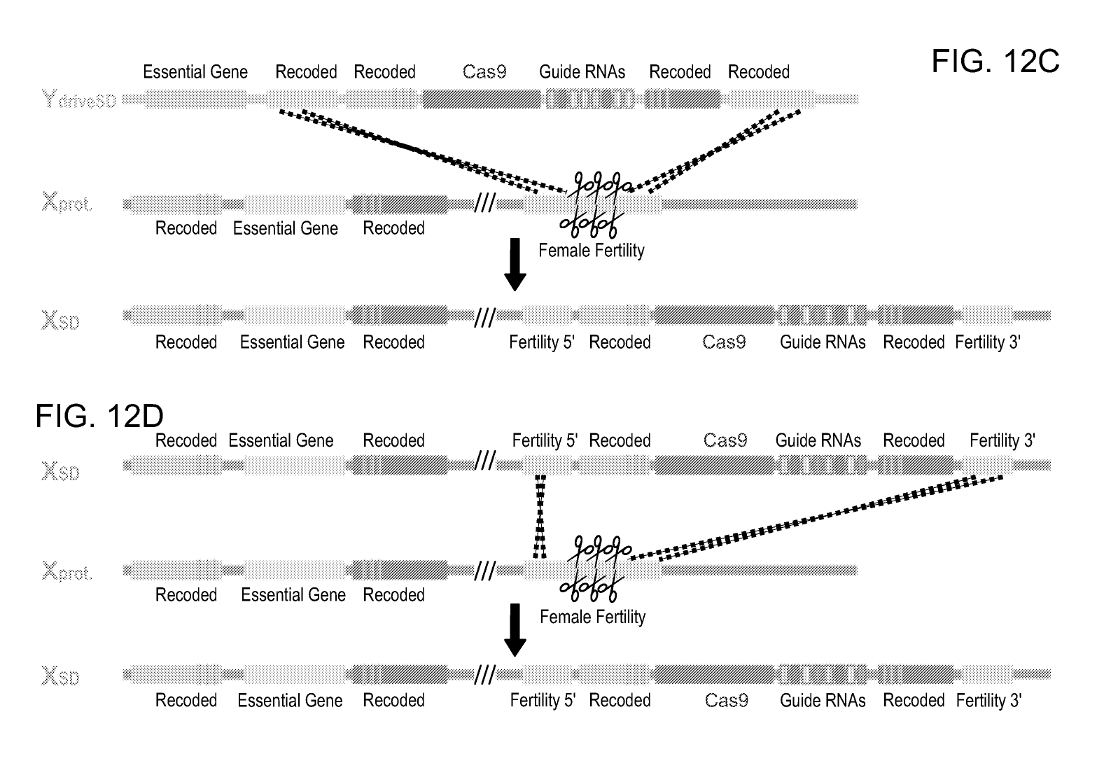

RNA-guided nucleases can also create less aggressive “sterile-daughter” Y chromosomes that can suppress populations but do not spread like a drive (FIG. 10A). A copy of Cas9 on the Y chromosome that targets an X-linked gene essential for female fertility rather than viability will still eliminate the reproductive potential of female progeny, but will provide no advantage to sons. Due to the minor cost of Cas9 activity, a sterile-daughter Y will eventually be outcompeted by the wild-type Y chromosome, but not before causing severe population suppression if introduced in sufficiently large numbers. This approach is closely related to the female-specific lethality and sterility methods being developed for the control of insect and fish populations, but is likely to be both more straightforward to construct and more evolutionarily stable due to its location on the Y.

Hybrid approaches to population control might combine the advantages of the Y-drive and sterile-daughter methods (FIG. 5A-B). For example, a Y chromosome that targets both an essential gene and a female fertility gene will quickly drive itself through the population because it is a Y-drive. However, it would lose its fitness advantage upon encountering a subsequently released standard gene drive that recodes the essential gene, but not the female fertility gene, on the X chromosome. Hybrid females will grow up to be infertile instead of dying embryonically, causing continued suppression without extinction.

A related approach to population control utilizes RNA-guided gene drives to disrupt one or more genes that are 1) required for fertility or viability, but 2) one intact copy largely suffices for function. Such a drive would cut and replace the target gene in the germline cells of organisms that inherit one copy of the drive and one copy of the wild-type gene at any time after the gene's function is required. For example, a gene required for gonad development and subsequent fertility might be cut and replaced by an RNA-guided gene drive just prior to meiosis. Since an organism that inherited only one copy would already have undergone correct gonad development because the wild-type copy suffices for this purpose, it would have normal fertility. However, most or all offspring would inherit the drive due to drive copying just prior to sperm and/or egg production. This design allows the drive to spread through the population when rare because most mating events will produce more individuals that inherit one copy of the drive and one copy of the wild-type. Later on, matings between two individuals that carry the drive will produce sterile offspring, leading to a population crash. This scenario has been modeled extensively as described in: Burt, A. Site-specific selfish genes as tools for the control and genetic engineering of natural populations. Proc. Biol. Sci. 270, 921-928 (2003). Deredec, A., Burt, A. & Godfray, H. C. J. The population genetics of using homing endonuclease genes in vector and pest management. Genetics 179, 2013-2026 (2008). North, A., Burt, A. & Godfray, H. C. J. Modelling the spatial spread of a homing endonuclease gene in a mosquito population. J. Appl. Ecol. 50, 1216-1225 (2013), each of which is hereby incorporated by reference in their entireties. Methods of restricting gene expression to germline cells and/or the time period prior to meiosis are known to those in the art.

### Example VI

### Applications

The gene drives described herein have particular practical utility in the eradication of infectious diseases and the control of invasive species. Such RNA guided Cas9 gene drives may be used to quickly spread protective alleles through threatened or soon-to-be-threatened species such as amphibians. Such RNA guided Cas9 gene drives may also be used to immunize wild populations that commonly serve as reservoirs for human viruses by targeting dsDNA viruses with Cas9 and RNA viruses with RNAi machinery from a foreign species carried by the drive. Disease vectors can be engineered to be unable to acquire the pathogen or can be eliminated using a Y drive described herein, i.e. an RNA guided Cas9 gene drive which inhibits propogation of an X chromosome. Similarly, invasive and ecologically destructive pests could be locally controlled or eradicated using Y-drives. Domesticated animals may be prevented from contributing genes to related wild species and feral populations controlled to minimize ecological damage and reduce the need for shelters and rescue agencies. Similarly, transgenic crops and animals could be genetically separated from their unmodified cousins, as could threatened species endangered by genetic dilution from more abundant or invasive relatives. Finally, gene drives could be used to directly test hypotheses concerning the evolutionary and ecological importance of genes, sex ratios, and speciation in natural environments.

The gene drives described herein have particular practical utility with vector born diseases. The human toll of vector-borne infectious disease is staggering. Malaria alone kills over 650,000 people each year, most of them children, while afflicting 200 million more with debilitating fevers that economically devastate their societies. Dengue, yellow fever, trypanosomiasis, leishmaniasis, Chagas disease, and Lyme disease are caused by others pathogens that spread using vectors. All of these can potentially be reduced or even eliminated by driving changes in the vector that prevent transmission. Scientists have identified several candidate gene disruptions or inserted genes that interfere with the transmission of malaria (Ito et al Nature 2002, PMID:12024215: Dong et al PloS Pathogens 2012, PMID: 22216006; Isaacs et al., PNAS 2012, PMID: 22689959) and other well-studied diseases (Franz et al PNAS 2006, PMID: 16537508), but not for many other pathogens. Hence, aspects of the present disclosure are directed to directly eliminating the vector species with a Y-drive. In the case of malaria, this strategy is particularly promising against emerging mosquito vectors that prefer to bite and rest outdoors, as these behaviors are highly resistant to current control strategies focused around indoor insecticide spraying and bednets. Although all vector species must be targeted in a given area in order to stop transmission, the disease will be permanently eradicated if the newly vacated ecological niches are filled by competing non-vector species. Significantly, this strategy requires little or no understanding of the vector's molecular biology, but unavoidably entails the local or possibly global extinction of the vector species.

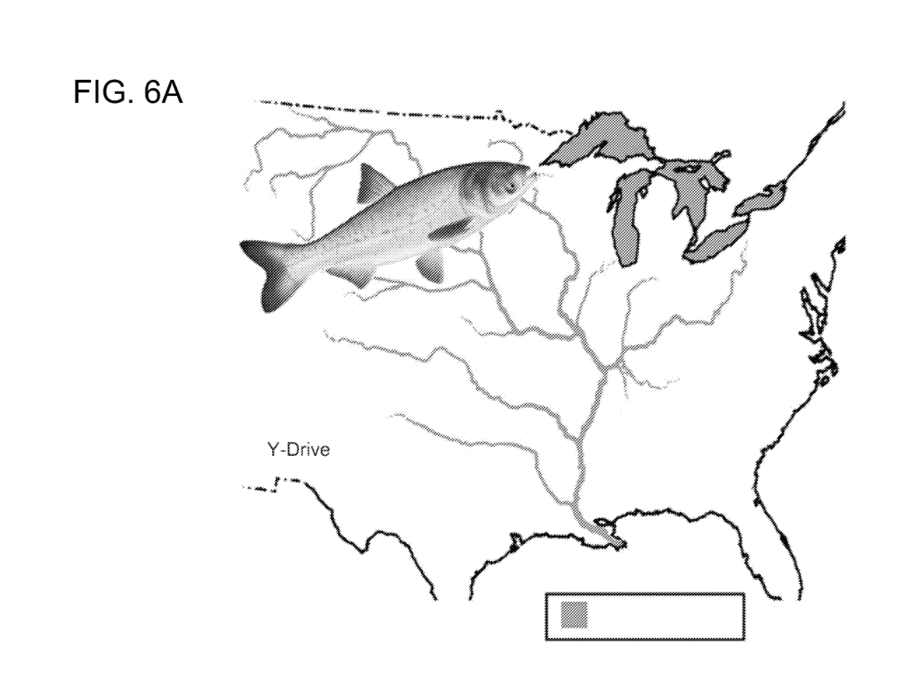

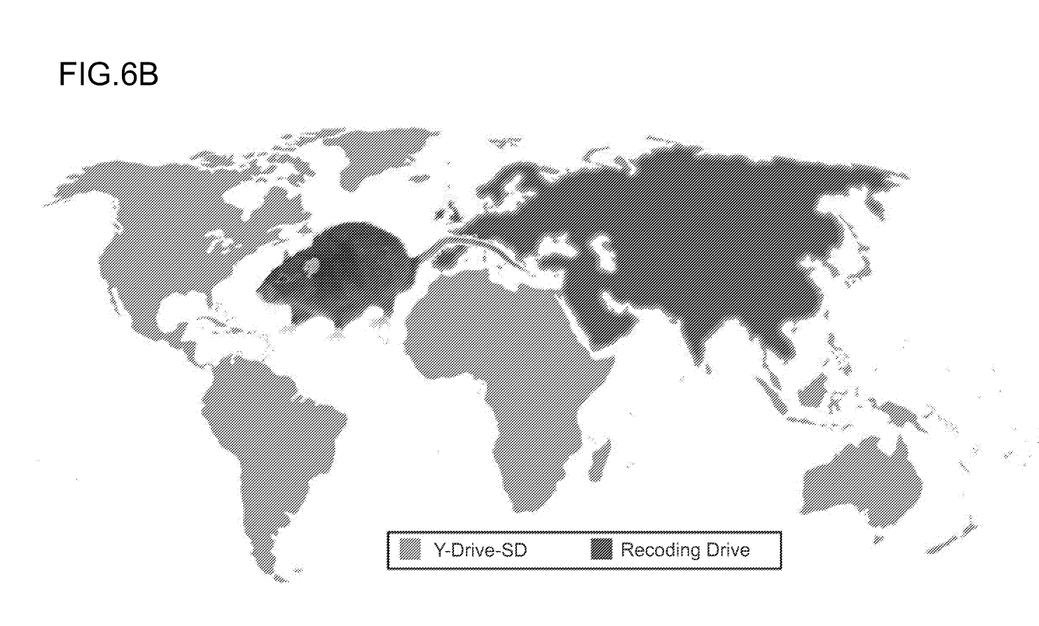

The gene drives described herein have particular practical utility in controlling invasive species. One of the most environmentally damaging consequences of global economic activity is the transport of invasive species, which often causes ecological disruption and the extinction of native species. Isolated ecosystems such as those on small islands are especially vulnerable. Cas9 Y-drives have tremendous potential to promote biodiversity by controlling or even eradicating these species from individual islands or possibly entire continents. Designing drive sequences unique to the invasive species, incorporating guide RNAs that will degrade the X chromosomes of at-risk relatives, and using speciation drives to render the target species genetically incompatible with its relatives target the drive to the selected species and reduce the risk of cross-species transfer without directly modifying all related species. Related species can be protected by releasing species-specific standard gene drives that recode the sites required for Y-drive function. The risk that the Y-drive might spread from the invasive population back into the native habitat is negligible for species that only invade through intentional human action, such as freshwater fish or cane toads, but Y-drive spread is all but certain for rats and other invasive stowaways. Native populations can always be protected from extinction by releasing an X-recoding drive or even a resistant X chromosome. As an example, invasive populations of low-mobility species such as Asian carp are excellent candidates for straightforward elimination by Y-drive. (FIG. 6A) Because gene flow would require deliberate human introduction, local eradication is likely to be permanent and achievable without being forced to recode the native population. In contrast, rats and other highly mobile invasive species cannot be permanently eliminated barring total extinction of the species. However, they might be controlled by periodically releasing a standard recoding drive in native populations and a Y-drive with sterile-daughter effect (Y-Drive-SD) throughout invasive populations (FIG. 6B).

### Example VII

### Agricultural Safety and Sustainability

Aspects of the present disclosure are directed to the use of the Cas9 methods described herein to control, reduce, or eliminate weeds and pests associated with agriculture. A pest generally refers to a plant or animal detrimental to humans or human concerns such as with agriculture or livestock production. The term pest may include any living organism which is invasive or prolific, detrimental, troublesome, noxious, destructive, a nuisance to either plants or animals, human or human concerns, livestock, human structures, wild ecosystems etc. The term pest may also include vermin, weed, plant and animal parasites and pathogens. A weed according to the present disclosure may refer to a plant considered undesirable in a particular situation. In a broad sense, a weed may be considered a pest that is a plant. Examples commonly are plants unwanted in human-controlled settings, such as farm fields, gardens, lawns, and parks. The term “weed” may also include any plant that grows or reproduces aggressively, or is invasive outside its native habitat.

According to one embodiment of the present disclosure, the Cas9 methods are used to insert into the genome of a germline cell of a weed or pest one or more RNA-guided “sensitizing drives.” A sensitizing drive is a gene drive as described herein and may also be referred to as a “sensitizing gene drive.” Accordingly, a “sensitizing drive” is a gene drive that is inserted into genomic DNA and which is transferred to progeny and renders the progeny sensitive to an external stimulus. According to one aspect, the RNA-guided “sensitizing drive” confers to the weed or pest as a result of being incorporated into genomic DNA a harmful sensitivity to a compound or chemical, such as toxicity. In this manner, the growth and proliferation of the weed or pest may be controlled by contacting the weed or pest with a compound or chemical or condition which ordinarily would not be toxic to the weed or pest. In this manner, the phenotype of the weed or pest has been altered by the sensitizing gene drive as a result of insertion of the gene drive into the germline cell and transfer to progeny. In this manner, the reference to a weed or pest may refer to the population that results from initial insertion of the sensitizing gene drive into the germline cell and mating with a wild type population. The resulting population may be referred to as an altered population or sensitized population or genetically altered population.

According to the present disclosure, the term “sensitivity” as it relates to a weed or pest means a harmful reaction, such as toxicity, to a compound or chemical or condition to which the weed or pest is exposed. The sensitizing drive is a gene drive as described herein that alters genomic DNA to result in a harmful reaction, such as toxicity, in response to a compound, chemical or condition to which the weed or pest is exposed. “Sensitizing drives” may also be referred to herein as “toxicity drives” or “toxicity gene drive” to the extent that a chemical, compound or condition is toxic to the weed or pest as a result of having the sensitizing drive or toxicity drive present in its genome. According to one aspect, the sensitizing drive or toxicity drive is exogenously added to the germline organism. In this manner, the sensitizing gene drive or toxicity gene drive is a foreign nucleic acid, even though, according to certain application, it may include a sequence native to the weed or pest species, but not present in the germline cell into which it is to be introduced. According to one aspect, the sensitizing drive or toxicity drive is a foreign nucleic acid that is exogenously added to the germline organism using an RNA guided DNA binding protein nuclease and associated guide RNA as described herein.

According to one aspect, the weed or pest may be resistant to a particular herbicide or pesticide or the weed or pest may have developed over time a resistance to the particular herbicide or pesticide. The evolution of weeds or pests to herbicides or pesticides is a major problem for agriculture. Methods described herein use RNA-guided “sensitizing drives” to confer sensitivity of the organism to a chemical, compound or condition that was previously nontoxic or of low toxicity to the organism, so that progeny of the organism will be sensitive to the chemical, compound or condition and the organism will die as a result of toxicity or proliferation will be lowered, or the organism will be rendered sterile so that it cannot reproduce. In this manner, a nucleic acid sequence within the sensitizing gene drive is expressed by the organism, such as a weed or pest, and the expression of the nucleic acid alters the phenotype of the organism rendering it vulnerable to the chemical, compound or condition.

According to an additional aspect, a sensitizing drive or drives are used to replace resistant alleles (such as those with resistance mutations) with their ancestral (or non-mutated) equivalents to restore sensitivity. Accordingly, an organism that has developed resistance to an herbicide or pesticide through mutation of an allele may be rendered sensitive to the herbicide or pesticide by removing the mutant form of the allele having resistance and replacing it with the non-mutant form having sensitivity. In this manner, the non-mutant form of the allele is foreign to the organism or population of organisms as it includes the mutant form of the allele. Accordingly, a sensitizing gene drive might reverse known mutations that confer resistance to existing pesticides or herbicides. RNA-guided gene drives are used to reverse genome alterations that have spread through populations of an organism. According to this aspect, releasing a second drive could overwrite one or all changes caused by a first drive, as described herein. In this manner, the population of organisms is characterized by the presence of a first gene drive and first nucleic acid sequence imparting a phenotype such as toxicity that was first introduced into a germline cell to produce that phenotype in progeny and which was then transferred to progeny. However, through mutations or otherwise, the phenotype is lost. In this context, a second gene drive with a second nucleic acid sequence imparting the same phenotype is introduced into a germline cell with or without the first nucleic acid being removed and the second nucleic acid sequence is carried through into progeny. In this manner, the first gene drive nucleic acid is overwritten by the second gene drive nucleic acid, for example, to result in toxicity, by reversing the mutation creating resistance.

According to an additional aspect, a sensitizing drive may carry a prodrug-converting enzyme that would render a prodrug molecule toxic to organisms that express the enzyme. In this manner, the enzyme is produced and should the organism be contacted with the prodrug, the enzyme will convert the prodrug into a compound or chemical that is toxic to the organism, such as a weed or pest. Various prodrug/enzyme combinations will be apparent to one of skill in the art based on the present disclosure.

According to a still additional aspect, a sensitizing drive may replace an essential gene for a version that is strongly inhibited by a particular small molecule. Accordingly, either expression of the gene would be inhibited or the expression product of the gene would be inhibited. Such inhibition may result in death of the organism or a lower proliferation. Because, in some embodiments, sensitizing drives would have no effect in the absence of their associated molecule—and in some cases vice versa—they could grant very fine control over the geography and extent of population suppression with minimal ecological risk.

According to one exemplary aspect, a sensitizing drive is used to reverse mutations allowing the western corn rootworm to resist Bt toxins (see Gassman et al PNAS, vol. 111 no. 14, pages 5141-5146, doi: 10.1073/pnas.1317179111 (2014) hereby incorporated by reference in its entirety) or horseweed and pigweed to resist the herbicide glyphosate (See Gaines, T. A. et al. Proc. Natl. Acad. Sci. 107, 1029-1034 (2010) and Ge, X., d'Avignon, D. A., Ackerman, J. J. & Sammons, R. D. Pest Manag. Sci. 66, 345-348 (2010) each of which are hereby incorporated by reference in their entireties), an herbicide currently essential to environmentally sustainable no-till agriculture. According to this aspect, organisms having the sensitizing drive are released into the wild population, individual organisms of which may or may not have resistance, and the sensitizing drive is carried through into progeny, the result of which is to decrease resistance of the progeny population (where resistance is present in the wild population) and increase sensitivity of the progeny population to a particular herbicide or pesticide (where resistance is present in the wild population.) It is to be understood that only some members of the wild type population may have resistance but that the sensitizing drive will spread through both sensitive and resistant members of the wild type population resulting in a progeny population having the sensitizing drive. According to one aspect, organisms including the sensitizing drive or drives would be released into areas not treated with herbicide or pesticide thereby creating reservoirs of sensitizing drives that could spread into adjacent areas which are treated with an herbicide or pesticide.

According to one aspect, methods are provided where a sensitizing drive or drives are used to counter mutations which lead to resistance. According to this aspect, introducing such sensitizing drives into the genome of a wild type population, initially and over subsequent time periods, that has developed resistance to a pesticide or herbicide as a result of a mutation or mutations reverses the effect of the mutation or mutations to provide resistance and renders the pesticide or herbicide toxic to the population. Such a method allows the use of any given herbicide or pesticide indefinitely because mutations conferring resistance are countered or replaced thereby “rolling back” whatever resistance may occur.

According to an additional aspect, a plant or animal may be rendered vulnerable to a chemical or compound or condition by the inclusion into its genome (and therefore its progeny) of a sensitizing drive or drives which renders the chemical, compound or condition toxic to the plant or animal, such as a weed or pest. Accordingly, compounds which may be considered safe to humans may be toxic to plants or animals as a result of the inclusion of a sensitizing drive or drives into the genome of the plant or animal thereby rendering the chemical or compound toxic to the plant or animal. According to this aspect, existing compounds considered safe and/or effective might be applied against organisms that are not presently vulnerable to them if a sensitizing drive were to deliver a sensitive gene from an affected species or laboratory isolate or replace a gene important for fitness with a sensitive gene.

According to a further aspect, methods are provided where sensitizing drives are used to render pest populations vulnerable to molecules, compounds or chemicals that are harmless to other forms of life. Whereas current pesticides and herbicides—even those designated as “organic”—are chosen for their toxicity towards insect pests and weeds, they often harm non-pest species or even humans because the affected pathways are conserved across species. According to the present disclosure, methods are provided where a gene or genes are delivered that confer sensitivity to an organism to a normally harmless molecule. Introduction of the sensitivity gene drive effectively converts that molecule into a pesticide or herbicide highly specific for the particular pest or weed species modified by the drive. The combination of the drive and the molecule is lethal to the organism. One exemplary embodiment is the use of an enzyme/prodrug combination where the enzyme is introduced in the genome of the organism and is expressed. When the organism is exposed to the prodrug, the enzyme converts the prodrug into an active herbicide or pesticide. Analogous candidates demonstrating the principle for this aspect of the present disclosure are antiviral or anticancer therapies in which a locally produced viral or tumor-specific enzyme activates a prodrug. Examples include the cytosine deaminase/5-fluorocytosine pairing and the nitroreductase/CB1954 pairing. In the case of sensitizing drives, the enzyme would be delivered to the target species by the gene drive, causing the prodrug to become a specific pesticide. Another exemplary pairing includes primary metabolic enzymes altered to become strongly inhibited by particular chemicals. For example, an engineered invertase may become nonfunctional in the presence of a mostly biologically inert xenobiotic chemical such as sucralose or related halogenated polysaccharides. A sensitizing drive would replace the organism's natural invertase gene with the engineered version, rendering it sensitive to the otherwise mostly inert compound. One of skill would readily be able to identify useful enzyme/chemical pairings suitable for this purpose based on the sensitizing drive methods described herein.

One of skill will readily be able to identify weeds within the scope of the present disclosure as including those weed plants harmful to agricultural crops. Such weeds may or may not be designated as “noxious weed” under federal or state law. For example, horseweed and pigweed are considered weeds that are harmful to agricultural crops but may not be designed as noxious weeds. Exemplary weeds designated as noxious weeds by the USDA include the following.

Aquatic

| Latin Name | Common Name(s) |
|---|---|
| Azolla pinnata | Mosquito fern, water velvet |
| Caulerpa taxifolia | Killer algae |
| (Mediterranean strain) | |
| Eichhornia azurea | Anchored waterhyacinth, rooted |
| | waterhyacinth |
| Hydrilla verticillata | Hydrilla |
| Hygrophila polysperma | Miramar weed |
| Ipomoea aquatica | Water-spinach, swamp morning glory |
| Lagarosiphon major | African elodea |
| Limnophila sessiliflora | Ambulia |
| Melaleuca quinquenervia | Broadleaf paper bark tree |
| Monochoria hastata | Arrowleaf false pickerelweed |
| Monochoria vaginalis | Heartshape false pickerelweed |
| Ottelia alismoides | Duck lettuce |
| Sagittaria sagittifolia | Arrowhead |
| Salvinia auriculata | Giant salvinia |
| Salvinia biloba | Giant salvinia |
| Salvinia herzogii | Giant salvinia |
| Salvinia molesta | Giant salvinia |
| Solanum tampicense | Wetland nightshade |
| Sparganium erectum | Exotic bur-reed |

Parasitic

| Latin Name | Common Name(s) |
|---|---|
| Aeginetia spp. | Varies by species |
| Alectra spp. | Varies by species |
| Cuscuta spp. (except for natives) | Dodders |
| Orobanche spp. (except for natives) | Broomrapes |
| Striga spp. | Witchweeds |

Terrestrial

| Latin Name | Common Name(s) |
|---|---|
| Acacia nilotica | Prickly acacia |
| Ageratina adenophora | Crofton weed |
| Ageratina riparia | Mistflower, spreading snakeroot |
| Alternanthera sessilis | Sessile joyweed |
| Arctotheca calendula | Capeweed |
| Asphodelus fistulosis | Onionweed |
| Avena sterilis | Animated oat, wild oat |
| Carthamus oxyacantha | Wild safflower |
| Chrysopogon aciculatus | Pilipiliula |
| Commelina benghalensis | Benghal dayflower |
| Crupina vulgaris | Common crupina |
| Digitaria scalarum | African couchgrass, fingergrass |
| Digitaria velutina | Velvet fingergrass, annual couchgrass |
| Drymaria arenariodes | Lightning weed |
| Emex australis | Three-corned jack |
| Emex spinosa | Devil's thorn |
| Euphorbia terracina | False caper, Geraldton carnation weed |
| Galega officinalis | Goatsrue |
| Heracleum mantegazzianum | Giant hogweed |
| Imperata brasiliensis | Brazilian satintail |
| Imperata cylindrica | Cogongrass |
| Inula britannica | British yellowhead |
| Ischaemum rugosum | Muraino grass |
| Leptochloa chinensis | Asian sprangletop |
| Lycium ferocissimum | African boxthorn |
| Lygodium flexuosum | Maidenhair creeper |
| Lygodium microphyllum | Old world climbing fern |
| Melastoma malabathricum | Malabar melastome |
| Mikania cordata | Mile-a-minute |
| Mikania micrantha | Bittervine |
| Mimosa invisa | Giant sensitive plant |
| Mimosa pigra | Catclaw mimosa |
| Moraea collina | Cape tulip |
| Moraea flaccida | One leaf cape tulip |
| Moraea miniata | Two leaf cape tulip |
| Moraea ochroleuca | Apricot tulip |
| Moraea pallida | Yellow tulip |
| Nassella trichotoma | Serrated tussock |
| Onopordum acaulon | Stemless thistle |
| Onopordum illyricum | Illyricum thistle |
| Opuntia aurantiaca | Jointed prickly pear |
| Oryza longistaminata | Red rice |
| Oryza punctata | Red rice |
| Oryza rufipogon | Red rice |
| Paspalum scrobiculatum | Kodo-millet |
| Pennisetum clandestinum | Kikuyugrass |
| Pennisetum macrourum | African feathergrass |
| Pennisetum pedicellatum | Kyasumagrass |
| Pennisetum polystachion | Missiongrass, thin napiergrass |
| Prosopis alpataco | Mesquite |
| Prosopis argentina | Mesquite |
| Prosopis articulata | Velvet mesquite |
| Prosopis burkartii | Mesquite |
| Prosopis caldenia | Calden |
| Prosopis calingastana | Cusqui |
| Prosopis campestris | Mesquite |
| Prosopis castellanosii | Mesquite |
| Prosopis denudans | Mesquite |
| Prosopis elata | Mesquite |
| Prosopis farcta | Syrian mesquite |
| Prosopis ferox | Mesquite |
| Prosopis fiebrigii | Mesquite |
| Prosopis hassleri | Mesquite |
| Prosopis humilis | Algaroba |
| Prosopis kuntzei | Mesquite |
| Prosopis pallida | Kiawe, algarroba |
| Prosopis palmeri | Mesquite |
| Prosopis reptans | Tornillo |
| Prosopis rojasiana | Mesquite |
| Prosopis ruizlealii | Mesquite |
| Prosopis ruscifolia | Mesquite |
| Prosopis sericantha | Mesquite |
| Prosopis strombulifera | Argentine screwbean |
| Prosopis torquata | Mesquite |
| Rottboellia cochinchinensis | Itchgrass |
| Rubus fruticosis | Wild blackberry |
| Rubus moluccanus | Wild raspberry |
| Saccharum spontaneum | Wild sugarcane |
| Sagittaria sagittifolia | Arrowhead |
| Salsola vermiculata | Wormleaf salsola |
| Senecio inaequidens | South African ragwort |
| Senecio madagascariensis | Fireweed |
| Setaria pumila | Cattail grass |
| Solanum torvum | Turkeyberry |
| Solanum viarum | Tropical soda apple |
| Spermacoce alata | Winged false buttonweed |
| Tridax procumbens | Coat buttons |
| Urochloa panicoides | Liverseed grass |

Additional weeds within the scope of the present disclosure include Atriplex, Spreading; Beggarsticks, Nodding; Brome, Downy; Carrot, wild; Chamomile, scentless; Chickweed, common; Cucumber, bur; Dandelion; Fleabane, Canada; Flixweed; Grass, Stink; Grass, Tufted love; Groundcherry, Smooth; Hedge-nettle, Marsh; Horse-nettle; Horsetail, Field; Lettuce, Prickly; Mercury, Three-seeded; Muhly, Wire-stemmed; Nipplewort; Redtop; S andbur, Long-spined; Smartweed, Swamp; Sow-thistle, Annual; Sow-thistle, Perennial; Speedwell, Corn; Vetch, Tufted; Violet, Field; Waterhemp, Common; Wood-sorrel species; Bermuda grass; Bindweed; Broadleaf plantain; Burdock; Common lambsquarters; Creeping Charlie; Dandelion; Goldenrod; Japanese Knotweed; Kudzu; Leafy spurge; Milk thistle; Poison ivy; Ragweed; Sorrel; Striga; St John's wort; Sumac; Tree of heaven; Wild carrot; Wood sorrel and Yellow nutsedge.

Additional weeds identified by scientific name include Acalypha rhomboidea Raf.; Agrostis gigantea Roth; Amaranthus rudis L.; Atriplex patula L.; Bidens cernua L.; Bromus tectorum L.; Cenchrus longispinus Hack.; Conyza Canadensis; Daucus carota L.; Descurainia sophia L.; Equisetium arvense L.; Eragrostis spp.; Lactuca scariola L.; Lapsana communis L.; Matricaria perforata Merat.; Muhlenbergia frondosa Poir.; Oxallis dillenii Jacq; Physalis virginiana Mill.; Polygonum coccineum Muhl.; Sicyos angulatus L.; Solanum carolinense L; Sonchus arvensis L.; Sonchus oleraceus L.; Stachys palustris L.; Stellaria media; Taraxacum officinale Weber.; Veronica avensis L.; Vicia cracca L.; and Viola avensis L.

It is to be understood that additional weeds within the scope of the present disclosure may be identified by sources readily available to those of skill in the art.

Common herbicides to which a weed may be resistant or develop resistance include the following. One of skill in the art will readily be able to identify herbicides toxic to particular weed species based on the present disclosure.

| Common or | | |
|---|---|---|
| Code Name* | Trade Name | Manufacturer |
| Acetochlor | Harness | Monsanto |
| Acifluorfen | Blazer | BASF |
| Aclonifen | Challenge | Bayer CropScience |
| Acrolein | Magnacide | Baker |
| Alachlor | Lasso | Monsanto |
| Alloxydim | Fervin | Bayer CropScience |
| Ametryn | Evik | Syngenta |
| Amicarbazone | | Bayer CropScience |
| Amidosulfuron* | Adret, Gratil | Bayer CropScience |
| Aminocyclopyrachlor | MAT28/KJM44 | DuPont |
| Aminopyralid | Milestone | Dow AgroSciences |
| Amitrole | Amitrol T | Bayer CropScience |
| Anilofos | Aniloguard | Ghardat |
| Asulam | Asulox | Bayer CropScience |
| Atrazine | Aatrex | Syngenta |
| | Atrazine | Terra |
| Azimsulfuron | Gulliver | DuPont |
| Beflubutamid | Herbaflex | Ube Industries |
| Benazolin | Asset | Bayer CropScience |
| Benefin | Balan | Dow AgroSciences |
| Bensulfuron | Londax | DuPont |
| Bensulide | Prefar | Gowan |
| Bentazon | Basagran | BASF |
| Benzofenap | Taipan | Bayer CropScience |
| Bifenox | Modown | Bayer CropScience |
| Bispyribac | Regiment, Velocity | Valent |
| Bromacil | Hyvar | DuPont |
| Bromoxynil | Buctril | Bayer CropScience |
| Butachlor | Machete | Monsanto |
| Butafenacil | Rebin, Inspire | Syngenta |
| Butroxydim | Falcon | Syngenta |
| Butylate | Sutan | Cedar |
| Cacodylic acid | Cotton-Aide HC | Monterey |
| Carbetamide | Carbetamex | Feinchemie |
| Carfentrazone | Aim, Shark, | FMC |
| | Quicksilver | |
| Chlorsulfuron | Glean, Telar | DuPont |
| Chlortoluron | Dicuran | Syngenta |
| Cinmethylin | Argold, Cinch | BASF |
| Clethodim | Select, Prism | Valent |
| Clodinfop | Topik | Syngenta |
| Clodinafop-propargy* | Discover | Syngenta |
| Clomazone | Command | FMC |
| Clopyralid | Stinger, Lontrel | Dow AgroSciences |
| Cloransulam-methyl | Firstrate | Dow AgroSciences |
| Cyanazine | Bladex | DuPont |
| Cycloate | RoNeet | Cedar |
| Cyclosulfamuron | Invest | BASF |
| Cycloxydim | Focus | BASF |
| Cyhalofop | Clincher | Dow AgroSciences |
| DCPA | Dacthal | Amvac |
| 2,4-D | Several | Several |
| 2,4-DB | Several | Several |
| Dazomet | Basamid | BASF |
| Desmedipham | Betamix | Bayer CropScience |
| Desmetryn | Semeron | Syngenta |
| Dicamba | Banvel, Clarity | BASF |
| Dichlobenil | Casoron | Uniroyal |
| Dichlorprop | Several | Bayer CropScience |
| Diclofop | Hoelon | Bayer CropScience |
| Diclosulam | Strongarm | Dow AgroSciences |
| Difenzoquat | Avenge | BASF |
| Diflufenican | Javelin | Bayer CropScience |
| Diflufenzopyr | Distinct | BASF |
| Dimethenamid | Frontier | BASF |
| Diquat | Reward, Reglone | Syngenta |
| Dithiopyr | Dimension | Monsanto |
| Diuron | Karmex | DuPont |
| DSMA | Several | several |
| Endothall | Several | Atochem |
| EPTC | Eptam | Syngenta |
| Ethalfluralin | Sonalan | Dow AgroSciences |
| | Curbit | UAP |
| Ethametsulfuron | Muster | DuPont |
| Ethofumesate | Nortron, Prograss | Bayer Crop Science |
| Ethoxysulfuron | Sunrice | Bayer Crop Science |
| Fenoxaprop | Acclaim, Whip | Bayer CropScience |
| Fentrazamide | Lecs | Bayer CropScience |
| Flazasulfuron | Katana | Ishihara Sangyo |
| Florasulam | Frontline, Boxer | Dow AgroSciences |
| Fluazifop-P | Fusilade DX, | Syngenta |
| | Fusilade II | |
| Flucarbazone-sodium | Everest | Arvesta |
| Flufenacet | Axiom (flufenacet + | Bayer CropScience |
| | metribuzin) | |
| | Epic (flufenacet + | |
| | isoxaflutole) | |
| Flufenpyr-ethyl* | S-3153 | Valent |
| Flumetsulam | Python, Broadstrike | Dow AgroSciences |
| Flumiclorac | Resource | Valent |
| Flumioxazin | BroadStar, Valor, | Valent |
| | Chateau | |
| Fluometuron | Cotoran | Syngenta |
| | Others | others |
| Flupyrsulfuron | Lexus | DuPont |
| Flurchloridone | Racer | Syngenta |
| Fluridone | Sonar | Dow AgroSciences |
| Fluroxypyr | Starane, Vista, | Dow AgroSciences |
| | Spotlight | |
| Fluthiacet | Action | Syngenta |
| Fomesafen | Reflex, Flexstar | Syngenta |
| Foramsulfuron | Equip, Revolver | Bayer CropScience |
| Fosamine | Krenite | Dupont |
| Glufosinate | Liberty, Rely | Bayer CropScience |
| Glyphosate | Roundup, Ultra, | Monsanto, |
| | Rodeo, Touchdown | Syngenta |
| | Pro | |
| Halosulfuron | Permit, Battalion | Monsanto |
| | SedgeHammer | Gowan |
| Haloxyfop | Verdict | Dow AgroSciences |
| Hexazinone | Velpar | DuPont |
| Imazametha benz | Assert | BASF |
| Imazamox | Raptor, Clearcast | BASF |
| Imazapic | Cadre, Plateau | BASF |
| Imazapyr | Arsenal, Habitat | BASF |
| Imazaquin | Image, Scepter | BASF |
| Imazethapyr | Pursuit | BASF |
| Iodosulfuron | Husar | Bayer CropScience |
| Isoxaben | Gallery | Dow AgroSciences |
| Isoxaflutole | Balance | Bayer CropScience |
| Lactofen | Cobra | Valent |
| Linuron | Lorox | DuPont |
| | Others | others |
| MCPA | Several | several |
| MCPB | Several | several |
| Mecoprop | MCPP, Mecopex | several |
| Mesosulfuron | Osprey | Bayer CropScience |
| Mefluidide | Enable | 3M Company |
| Mesotrione | Callisto, Tenacity | Syngenta |
| Metham | Metham Sodium | Amvac |
| Metolachlor | Dual | Syngenta |
| | Dual Magnum, | |
| | Pennant Magnum | |
| Metribuzin | Sencor, Lexone | Bayer CropScience |
| Metsulfuron | Ally, Escort | DuPont |
| MSMA | Several | several |
| Napropamide | Devrinol | United Phosphorus |
| Naptalam | Alanap | Uniroyal |
| Nicosulfuron | Accent | DuPont |
| Norflurazon | Zorial, Solicam | Syngenta |
| Oryzalin | Surflan | Dow AgroSciences |
| Oxadiazon | Ronstar | Lesco |
| Oxasulfuron* | Expert | Syngenta |
| Oxyfluorfen | Goal | Dow AgroSciences |
| Paraquat | Gramoxone Inteon, | Syngenta |
| | Gramoxone Max | |
| Pebulate | Tillam | Monterey |
| Pelargonic acid | Scythe | Dow AgroSciences |
| Pendimethalin | Prowl | BASF |
| Penoxsulam | Grasp, Granite | Dow AgroSciences |
| Phenmedipham | Spin-Aid | Bayer CropScience |
| Picloram | Tordon | Dow AgroSciences |
| Picolinafen* | Pico | BASF |
| Pinoxaden | Axial | Syngenta |
| Primisulfuron | Beacon | Syngenta |
| Prodiamine | Barricade | Syngenta |
| Prometon | Pramitol | Agriliance |
| Prometryn | Caparol | Syngenta |
| Pronamide | Kerb | Dow AgroSciences |
| Propanil | Siam | Dow AgroSciences |
| Propaquizafop | Shogun | Syngenta |
| Propoxycarbazone | Olympus | Bayer CropScience |
| Propazine | MiloPro | Griffin |
| Prosulfocarb | Boxer (in France) | Syngenta |
| Pyrasulfotole | Huskie | Bayer CropScience |
| Pyrazon | Pyramin | BASF |
| Pyrazosulfuron-ethyl* | Sirius | Bayer CropScience |
| Pyridate | Tough | Syngenta |
| Pyrithiobac | Staple | DuPont |
| Pyroxsulam | Powerflex | Dow AgroSciences |
| Quinclorac | Drive, Facet, | BASF |
| | Paramount | |
| Quinmerac* | Fiesta | BASF |
| Quizalofop | Assure | DuPont |
| Rimsulfuron | Matrix, Basis, | DuPont |
| | TranXit | |
| Saflufenacil | Kixor | BASF |
| Sethoxydim | Poast | BASF |
| | Poast Plus | |
| Siduron | Tupersan | DuPont |
| Simazine | Princep, others | Syngenta, others |
| Sulcotrione | Mikado, Galleon | Bayer CropScience |
| Sulfentrazone | Dismiss, Spartan, | FMC |
| | Authority | |
| Sulfometuron | Oust | DuPont |
| Sulfosulfuron | Certainty, Monitor, | Monsanto |
| | Outrider, Maverick | |
| Tebuthiuron | Spike | Dow AgroSciences |
| Tembotrione | Laudis | Bayer CropScience |
| Tepaloxydim | Equinox | BASF |
| Terbacil | Sinbar | DuPont |
| Terbutryn | Igran | Syngenta |
| Thiazopyr | Visor | Dow AgroSciences |
| Thifensulfuron | Pinnacle, Harmony | DuPont |
| Thiencarbazone | | Bayer CropScience |
| Thiobencarb | Bolero | Valent |
| Topramezone | Impact | Amvac |
| Tralkoxydim | Achieve | Syngenta |
| Triallate | FarGo | Monsanto |
| Triasulfuron | Amber, Fuego | Syngenta |
| Tribenuron | Express | DuPont |
| Triclopyr | Renovate | SeaPro |
| | Garlon, | Dow AgroSciences |
| Trifloxysulfuron | Envoke, Monument | Syngenta |
| Triflusulfuron | Upbeet | DuPont |
| Vernolate | Vernam | Drexel |

It is to be understood that additional herbicides within the scope of the present disclosure may be identified by sources readily available to those of skill in the art.

Pests within the scope of the present disclosure associated with corn include the following.

| Scientific Name | Common Name |
|---|---|
| Autographa gamma | Silver Y moth |
| Chilo suppressalis | Asiatic rice borer |
| Diabrotica speciosa | Cucurbit beetle |
| Harpophora maydis | Late wilt of corn |
| Helicoverpa armigera | Old world bollworm |
| Heteronychus arator | Black maize beetle |
| Peronosclerospora maydis | Java downy mildew |
| Peronosclerospora philippinensis | Philippine downy mildew |
| Punctodera chalcoensis | Mexican corn cyst nematode |
| Sclerophthora rayssiae var. zeae | Brown stripe downy mildew |
| Spodoptera littoralis | Egyptian cottonworm |
| Spodoptera litura | Cotton cutworm |
| Thaumatotibia leucotreta | False codling moth |

Pests within the scope of the present disclosure associated with cotton include the following.

| Scientific Name | Common Name |
|---|---|
| Anthonomus grandis | Boll weevil |
| Autographa gamma | Silver Y moth |
| Eutetranychus orientalis | Citrus brown mite |
| Helicoverpa armigera | Old World bollworm |
| Oxycarenus hyalinipennis | Cotton seed bug |
| Pectinophora gossypiella | Pink bollworm |
| Spodoptera littoralis | Egyptian cottonworm |
| Spodoptera litura | Cotton cutworm |
| Thaumatotibia leucotreta | False codling moth |

Pests within the scope of the present disclosure associated with oak include the following.

| Scientific Name | Common Name |
|---|---|
| Adoxophyes orana | Summer fruit tortrix moth |
| Aeolesthes sarta | City longhorned beetle |
| Agrilus biguttatus | Oak splendour beetle |
| Archips xylosteanus | Variegated golden tortrix |
| Epiphyas postvittana | Light brown apple moth |
| Lymantria dispar asiatica | Asian gypsy moth |
| Lymantria mathura | Rosy moth |
| Massicus raddei | Mountain oak longhorned beetle |
| Phytophthora quercina | Oak decline |
| Platypus quercivorus | Oak ambrosia beetle |
| Raffaelea quercivora | Japanese oak wilt |
| Scolytus intricatus | European oak bark beetle |
| Spodoptera littoralis | Egyptian cottonworm |
| Thaumatotibia leucotreta | False codling moth |
| Thaumetopoea processionea | Oak processionary moth |
| Tortrix viridana | Green oak tortrix |
| Tremex fuscicornis | Tremex woodwasp |

Pests within the scope of the present disclosure associated with pine include the following.

| Scientific Name | Common Name |
|---|---|
| Candidatus Phytoplasma | Pine witches' broom |
| pini 16SrXXI-A | |
| Cronartium flaccidum | Scots pine blister rust |
| Dendroctonus micans | Great spruce bark beetle |
| Dendrolimus pini | Pine-tree lappet |
| Dendrolimus punctatus | Masson pine moth |
| Dendrolimus sibiricus | Siberian silk moth |
| Diprion pini | Pine sawfly |
| Hylobius abietis | Large pine weevil |
| Lymantria mathura | Rosy moth |
| Monochamus saltuarius | Japanese pine sawyer |
| Monochamus sutor | Small white-marmorated longhorned beetle |
| Mycosphaerella gibsonii | Needle blight of pine |
| Panolis flammea | Pine beauty moth |
| Tomicus destruens | No common name, a pine shoot beetle |

Pests within the scope of the present disclosure associated with small grains include the following.

| Scientific Name | Common Name |
|---|---|
| Autographa gamma | Silver Y moth |
| Cernuella virgata | Maritime garden snail |
| Cochlicella spp. | Exotic species |
| Diabrotica speciosa | Cucurbit beetle |
| Helicoverpa armigera | Old world bollworm |
| Heterodera filipjevi | Cereal cyst nematode |
| Heterodera latipons | Mediterranean cereal cyst nematode |
| Heteronychus arator | Black maize beetle |
| Lobesia botrana | European grape vine moth |
| Meloidogyne artiellia | British root-knot nematode |
| Nysius huttoni | Wheat bug |
| Peronosclerospora philippinensis | Philippine downy mildew |
| Spodoptera littoralis | Egyptian cottonworm |
| Spodoptera litura | Cotton cutworm |

Pests within the scope of the present disclosure associated with soybean include the following.

| Scientific Name | Common Name |
|---|---|
| Adoxophyes orana | Summer fruit tortrix moth |
| Alectra vogelii | Yellow witchweed |
| Autographa gamma | Silver Y moth |
| Cernuella virgata | Maritime garden snail |
| Chrysodeixis chalcites | Golden twin spot moth |
| Crocidosema aporema | Bud borer |
| Diabrotica speciosa | Cucurbit beetle |
| Eutetranychus orientalis | Citrus brown mite |
| Helicoverpa armigera | Old world bollworm |
| Spodoptera littoralis | Egyptian cottonworm |

Pests within the scope of the present disclosure associated with grape include the following.

| Scientific Name | Common Name |
|---|---|
| Adoxophyes orana | Summer fruit tortrix moth |
| Autographa gamma | Silver Y moth |
| Candidatus Phytoplasma australiense | Australian grapevine yellows |
| 16SrXII-B | |
| Cryptoblabes gnidiella Epiphyas | Honeydew moth |
| postvittana Eupoecilia ambiguella | |
| Candidatus Phytoplasma vitis 1 | Light brown apple moth |
| Heteronychus arator Lobesia botrana | European grape berry moth |
| Pseudopezicula tracheiphila Spodoptera | Flavescence doree Black |
| | maize |
| littoralis Spodoptera litura Thaumatotibia | beetle European grape vine |
| leucotreta 6SrV-C | moth Rothrenner |

Pests within the scope of the present disclosure associated with palm include the following.

| Scientific Name | Common Name |
|---|---|
| Bursaphelenchus cocophilus | Red ring nematode |
| Candidatus Phytoplasma palmae | Palm lethal yellowing |
| 16SrIV | |
| Cocadviroid Coconut cadang cadang | Coconut cadang cadang |
| viroid | |
| Darna pallivitta | Nettle caterpillar |
| Haplaxius crudus | American palm cixiid |
| Metamasius hemipterus | West Indian cane weevil |
| Oryctes rhinoceros | Coconut rhinoceros beetle |
| Paysandisia archon | No common name, a palm borer |
| Raoiella indica | Red palm mite |
| Rhabdoscelus obscurus | New Guinea sugarcane weevil |
| Rhynchophorus ferrugineus | Red palm weevil |
| Rhynchophorus palmarum | South American palm weevil |

Pests within the scope of the present disclosure associated with solanaceous plants include the following.

| Scientific Name | Common Name |
|---|---|
| Autographa gamma | Silver-Y moth |
| Candidatus Phytoplasma | Australian grapevine yellows |
| australiense 16SrXII-B | |
| Chrysodeixis chalcites | Golden twin spot moth |
| Globodera pallida | Pale cyst nematode |
| Globodera rostochiensis | Golden nematode |
| Helicoverpa armigera | Old world bollworm |
| Meloidogyne fallax | False Columbia root-knot nematode |
| Meloidogyne minor | Root-knot nematode |
| Neoleucinodes elegantalis | Tomato fruit borer |
| Ralstonia solanacearum race 3 | Bacterial wilt/Southern bacterial Wilt |
| biovar 2 | |
| Spodoptera littoralis | Egyptian cottonworm |
| Spodoptera litura | Cotton cutworm |
| Synchytrium endobioticum | Potato wart |
| Tecia solanivora | Guatemalan potato tuber moth |
| Thaumatotibia leucotreta | False codling moth |
| Tuta absoluta | Tomato leaf miner |

Pests within the scope of the present disclosure associated with stone fruit include the following.

| Scientific Name | Common Name |
|---|---|
| Adoxophyes orana | Summer fruit tortrix |
| Argyresthia pruniella | Cherry blossom moth |
| Bactrocera zonata | Peach fruit fly |
| Candidatus Phytoplasma prunorum | European stone fruit yellows |
| 16SrX-F | |
| Enarmonia formosana | Cherry bark tortrix |
| Epiphyas postvittana | Light brown apple moth |
| Grapholita funebrana (Syn.: Cydia | Plum fruit moth |
| funebrana) | |
| Leucoptera malifoliella | Pear leaf blister moth |
| Lobesia botrana | European grape vine moth |
| Monilia polystroma | Asiatic brown rot |
| Monilinia fructigena | Brown rot, Apple brown rot |
| Potyvirus Plum Pox Virus | Plum pox |
| Rhagoletis cerasi | European cherry fruit fly |
| Thaumatotibia leucotreta | False codling moth |

Additional agricultural pests include the following cyst nematodes.

| Scientific Name | Common Name |
|---|---|
| Globodera pallida | Pale cyst nematode |
| Globodera rostochiensis | Golden nematode |
| Heterodera cajani | Pigeonpea cyst nematode |
| Heterodera ciceri | Chickpea cyst nematode |
| Heterodera filipjevi | Cereal cyst nematode |
| Heterodera latipons | Mediterranean cereal cyst nematode |
| Heterodera sacchari | Sugarcane cyst nematode |
| Punctodera chalcoensis | Mexican corn cyst nematode |

Additional agricultural pests include the following exotic wood borer or bark beetles.

| Scientific Name | Common Name |
|---|---|
| Agrilus auroguttatus | Goldspotted oak borer |
| Agrilus biguttatus | Oak splendour beetle |
| Agrilus planipennis | Emerald ash borer |
| Anoplophora chinensis | Citrus longhorned beetle |
| Anoplophora glabripennis | Asian longhorned beetle |
| Chlorophorus annularis | Bamboo borer |
| Chlorophorus strobilicola | Slender-banded pine cone longhorn beetle |
| Dendroctonus micans | Great spruce bark beetle |
| Ips sexdentatus | Six-toothed bark beetle |
| Ips typographus | European spruce bark beetle |
| Megaplatypus mutatus | No common name, an ambrosia beetle |
| Monochamus alternatus | Japanese pine sawyer |
| Monochamus saltuarius | Japanese pine sawyer |
| Monochamus sutor | Small white-marmorated longhorned beetle |
| Orthotomicus erosus | Mediterraneran pine engraver |
| Pityogenes chalcographus | Sixtoothed spruce bark beetle |
| Platypus quercivorus | Oak ambrosia beetle |
| Scolytus intricatus | European oak bark beetle |
| Tetropium castaneum | Black spruce beetle |
| Tetropium fuscum | Brown spruce longhorned beetle |
| Tomicus destruens | No common name, a pine shoot beetle |
| Tomicus minor | Lesser pine shoot beetle |
| Tomicus piniperda | Pine shoot beetle |
| Trichoferus campestris | Velvet longhorned beetle |
| Trypodendron | European hardwood ambrosia beetle |
| domesticum | Redbay ambrosia beetle |

Additional agricultural pests include the following mollusks.

| Scientific Name | Common Name |
|---|---|
| Belocaulus spp. | No common name, leatherleaf slugs |
| Cernuella spp. | No common name, hygromiid snails |
| Cochlicella spp. | No common name, cochlicellid snails |
| Colosius spp. | No common name, leatherleaf slugs |
| Laevicaulis spp. | No common name, leatherleaf slugs |
| Lissachatina fulica | Giant African snail |
| Meghimatium pictum | Chinese slug |
| Monacha spp. | No common name, hygromiid snails |
| Sarasinula spp. | No common name, leatherleaf slugs |
| Semperula spp. | No common name, leatherleaf slugs |
| Veronicella spp. | No common name, leatherleaf slugs |

Additional agricultural pests include the following moths.

| Scientific Name | Common Name |
|---|---|
| Dendrolimus pini | Pine-tree lappet |
| Dendrolimus punctatus | Masson pine moth |
| Dendrolimus sibiricus | Siberian silk moth |
| Lymantria albescens | Okinawa gypsy moth |
| Lymantria dispar asiatica | Asian gypsy moth |
| Lymantria dispar japonica | Japanese gypsy moth |
| Lymantria mathura | Rosy moth |
| Lymantria monacha | Nun moth |
| Lymantria postalba | White-winged gypsy moth |
| Lymantria umbrosa | Hokkaido gypsy moth |
| Lymantria xylina | Casuarina tussock moth |

It is to be understood that additional pests within the scope of the present disclosure may be identified by sources readily available to those of skill in the art.

Common pesticides in general within the scope of the present disclosure to which a pest may be resistant or develop resistance include the following: algicides, antifouling agents, antimicrobials, attractants, biopesticides, biocides, disinfectants, fungicides, fumigants, insecticides, miticides, microbial pesticides, molluscicides, nematicides, pheromones, repellants and rodenticides.

The following pesticide species are useful within the scope of the present disclosure: Glyphosate, Atrazine, Metam Sodium, Metolachlor-S, Acetochlor, Dichloropropene, 2,4-D, Methyl Bromide, Chloropicrin, Pendimenthalin, Ethephon, Chlorothalonil, Metam Potassium, Chlorpyrifos, Copper Hydroxide, Copper Sulfate, Simazine, Trifluralin, Propanil, Mancozeb, Aldicarb, Acephate, Diuron, MCPA, Paraquat, Dimethenamid, Carbaryl, MCPP, MSMA, Pyrethroids, Malathion, Dicamba, Pelarganoc Acid, Sulfuryl fluoride, Triclopyr, Paradiclorobenzene, Naphthalene, Chlorpyrifos, Naled, Dicrotophos, Phosmet, Phorate, Diazinon, Dimethoate, Azinphos-Methyl, and N,N-diethyl-meta-toluamide (insect repellant). One of skill in the art will readily be able to identify additional pesticide species using publicly available information of databases, for example the EPA list of registered pesticides available at world wide website iaspub.epa.gov/apex/pesticides/f?p=chemicalsearch:1 and with reference to the most commonly used conventional pesticide active ingredients in the U.S. Agricultural, Home and Garden, Industry, Commercial, and Government Market Sectors as compiled by the EPA and publicly available at world wide website www.epa.gov/opp00001/pestsales/.

One of skill in the art will readily be able to identify pesticides toxic to particular pest species based on the present disclosure.

Accordingly, aspect of the present disclosure are directed to a method of altering a eukaryotic germline cell of an organism including introducing into the germline cell a first foreign nucleic acid sequence encoding an RNA guided DNA binding protein nuclease and one or more guide RNAs, and including corresponding promoter sequences and a first flanking sequence and a second flanking sequence, and including a sensitizing nucleic acid the expression of which is harmful to the organism when the organism is exposed to a chemical, compound or condition, wherein the one or more guide RNAs are complementary to one or more target locations on genomic DNA of a first chromosome and a second chromosome of a chromosome pair of the germline cell, wherein the nucleic acid sequence encoding the RNA guided DNA binding protein nuclease and the nucleic acid sequence encoding the one or more guide RNAs are between the first flanking sequence and the second flanking sequence, wherein the first flanking sequence includes a first sequence identical to a first portion of the target location on the first chromosome or the second chromosome of the genomic DNA, wherein the second flanking sequence includes a second sequence identical to a second portion of the target location on the first chromosome or the second chromosome of the genomic DNA, expressing the first foreign nucleic acid sequence to produce the RNA guided DNA binding protein nuclease and the one or more RNAs wherein the RNA guided DNA binding protein nuclease and an associated guide RNA co-localize to an associated target location on the first chromosome of the genomic DNA and the second chromosome of the genomic DNA and the RNA guided DNA binding protein nuclease cleaves the first chromosome of the genomic DNA at the target location in a cleavage site specific manner and cleaves the second chromosome of the genomic DNA at the target location in a cleavage site specific manner, inserting the first foreign nucleic acid sequence into the first chromosome of the chromosome pair of the genomic DNA at the cleavage site, and inserting the first foreign nucleic acid sequence into the second chromosome of the chromosome pair of the genomic DNA at the cleavage site to render the germline cell homozygous for the foreign nucleic acid sequence, and expressing the sensitizing nucleic acid rendering the resulting organism sensitive to the chemical, compound or condition such that the resulting organism perishes or is rendered sterile when exposed to the chemical, compound or condition.

According to one aspect, expression of the sensitizing nucleic acid increases toxicity of the chemical, compound or condition to the organism. According to one aspect, the germline cell is grown into an organism and the sensitizing nucleic acid is transferred to progeny to create a population of organisms including the sensitizing nucleic acid and where the sensitizing nucleic acid increases toxicity of the chemical, compound or condition to the organism. According to one aspect, the organism is a weed or pest. According to one aspect, the sensitizing nucleic acid is a sensitizing gene which replaces an existing gene. According to one aspect, the sensitizing gene is the exact or codon-altered ancestral version of an existing mutant gene in wild populations, such that the current mutated version is replaced with the ancestral form. According to one aspect, the existing gene has acquired a mutation contributing to resistance to a pesticide, herbicide, or fungicide. According to one aspect, the chemical or compound is a pesticide, herbicide or fungicide. According to one aspect, the pesticide, herbicide, or fungicide is one of: a Bt toxin produced by Cry1A.105, CryIAb, CryIF, Cry2Ab, Cry3Bb1, Cry34Ab1, Cry35Ab1, mCry3A, or VIP, 2,4-D or glyphosate. According to one aspect, the sensitizing gene replaces an existing gene whose function is required for the organism to survive or reproduce. According to one aspect, the chemical is a prodrug and the sensitizing gene encodes a corresponding prodrug-converting enzyme. According to one aspect, the enzyme/chemical pairing is cytosine deaminase/5-fluorocytosine, or nitroreductase/CB1954.

According to certain aspect of the present disclosure, a method of controlling a weed or pest population including a sensitivity gene drive in the genome of the weed or pest population wherein the sensitivity gene drive renders the weed or pest population vulnerable to toxicity when in the presence of a chemical, compound or condition is provided including contacting the weed or pest population with the chemical, compound or condition in an effective amount to kill the weed or pest, lower proliferation of the weed or pest or render the weed or pest sterile to inhibit proliferation. According to one aspect, the chemical or compound is an herbicide, or pesticide or fungicide.

According to certain aspect of the present disclosure, a method of altering a eukaryotic germline cell of an organism including a first sensitizing gene drive is provided including introducing into the germline cell a second foreign nucleic acid sequence encoding an RNA guided DNA binding protein nuclease and one or more guide RNAs, and including corresponding promoter sequences and a first flanking sequence and a second flanking sequence, and including a second sensitizing nucleic acid sequence the expression of which is harmful to the organism when the organism is exposed to a chemical, compound or condition, wherein the one or more guide RNAs are complementary to one or more target locations on genomic DNA of a first chromosome including the first sensitizing gene drive and a second chromosome of a chromosome pair of the germline cell including the first sensitizing gene drive, wherein the nucleic acid sequence encoding the RNA guided DNA binding protein nuclease and the nucleic acid sequence encoding the one or more guide RNAs of the second foreign nucleic acid sequence are between the first flanking sequence and the second flanking sequence, wherein the first flanking sequence includes a first sequence identical to a first portion of the target location on the first chromosome or the second chromosome of the genomic DNA, wherein the second flanking sequence includes a second sequence identical to a second portion of the target location on the first chromosome or the second chromosome of the genomic DNA, expressing the second foreign nucleic acid sequence to produce the RNA guided DNA binding protein nuclease and the one or more RNAs wherein the RNA guided DNA binding protein nuclease and an associated guide RNA co-localize to an associated target location on the first chromosome of the genomic DNA and the second chromosome of the genomic DNA and the RNA guided DNA binding protein nuclease cleaves the first chromosome of the genomic DNA at the target location in a cleavage site specific manner and cleaves the second chromosome of the genomic DNA at the target location in a cleavage site specific manner, wherein the first foreign nucleic acid sequence is removed, inserting the second foreign nucleic acid sequence into the first chromosome of the chromosome pair of the genomic DNA at the cleavage site, and inserting the second foreign nucleic acid sequence into the second chromosome of the chromosome pair of the genomic DNA at the cleavage site to render the germline cell homozygous for the second foreign nucleic acid sequence, and expressing the second sensitizing nucleic acid sequence rendering the resulting organism sensitive to the chemical, compound or condition such that the resulting organism perishes or is rendered sterile when exposed to the chemical, compound or condition. According to one aspect, the resulting organism is introduced into a wild type population such as that progeny of the resulting organism and a wild type organism includes the second sensitizing nucleic acid sequence. According to one aspect, the organism is a weed or pest.

According to certain aspect of the present disclosure, a method of altering a eukaryotic germline cell of an organism including a first sensitizing gene drive is provided including introducing into the germline cell a second foreign nucleic acid sequence encoding an RNA guided DNA binding protein nuclease and one or more guide RNAs, and including corresponding promoter sequences and a first flanking sequence and a second flanking sequence, and including a second sensitizing nucleic acid sequence the expression of which is harmful to the organism when the organism is exposed to a chemical, compound or condition, wherein the one or more guide RNAs are complementary to one or more target locations on genomic DNA of a first chromosome including the first sensitizing gene drive and a second chromosome of a chromosome pair of the germline cell including the first sensitizing gene drive, wherein the nucleic acid sequence encoding the RNA guided DNA binding protein nuclease and the nucleic acid sequence encoding the one or more guide RNAs of the second foreign nucleic acid sequence are between the first flanking sequence and the second flanking sequence, wherein the first flanking sequence includes a first sequence identical to a first portion of the target location on the first chromosome or the second chromosome of the genomic DNA, wherein the second flanking sequence includes a second sequence identical to a second portion of the target location on the first chromosome or the second chromosome of the genomic DNA, expressing the second foreign nucleic acid sequence to produce the RNA guided DNA binding protein nuclease and the one or more RNAs wherein the RNA guided DNA binding protein nuclease and an associated guide RNA co-localize to an associated target location on the first chromosome of the genomic DNA and the second chromosome of the genomic DNA and the RNA guided DNA binding protein nuclease cleaves the first chromosome of the genomic DNA at the target location in a cleavage site specific manner and cleaves the second chromosome of the genomic DNA at the target location in a cleavage site specific manner, inserting the second foreign nucleic acid sequence into the first chromosome of the chromosome pair of the genomic DNA at the cleavage site, and inserting the second foreign nucleic acid sequence into the second chromosome of the chromosome pair of the genomic DNA at the cleavage site to render the germline cell homozygous for the second foreign nucleic acid sequence, and expressing the second sensitizing nucleic acid sequence rendering the resulting organism sensitive to the chemical, compound or condition such that the resulting organism perishes or is rendered sterile when exposed to the chemical, compound or condition. According to certain aspects, the resulting organism is introduced into a wild type population such as that progeny of the resulting organism and a wild type organism includes the second sensitizing nucleic acid sequence. According to certain aspects, the organism is a weed or pest.

### Example VIII

### Plasmids and Genomic Cassettes

Gene drive cassettes were synthesized from gBlocks (Integrated. DNA Technologies, Coralville, Iowa) and inserted into SK1 cells via Cas9-mediated genome modification as follows. Guide RNAs for each drive were cloned into p416-Cas9 containing plasmids with expression driven by the SNR52 promoter. See DiCarlo, J. E. et al. Genome engineering in Saccharomyces cerevisiae using CRISPR-Cas systems, Nucleic Acids Res. 41, 4336-4343 (2013). 60 base pair homology arms to the target locus were added on both ends of the gene drive cassette via PCR and 5 ug of PCR product was co-transformed with the p416-Cas9-gRNA plasmids. Correctly integrated gene drives were verified by sequencing and p416-Cas9-gRNA plasmids were cured using 5-Fluoroorotic Acid (FOA) selection.

To create the URA3-containing ADE2 gene drive, the 0.40E2 gene drive was cloned next to the Candida albicans URA3 gene in the pAG60 plasmid. The entire URA3 cassette and gene drive were PCR amplified and inserted using Cas9-mediated genome modification into the ADE2 locus of haploid SK1 cells.

The recoded C-terminus of the ABD1 gene and corresponding gene drive were synthesized as a gBlock to remove homology and generate mutations in the seed sequence via synonymous changes. The TEF1 terminator was inserted at the 3′end of the recoded ABD1 gene between the gene and the gRNA as ABD1 shares a terminator with the MCI gene. The entire cassette was integrated into the haploid SK1 genome using Cas9-mediated genome modification.

The p416-Cas9-gRNA plasmid (conferring uracil prototrophy) is a variant of the previously described p414-Cas9-gRNA plasmid (conferring tryptophan prototrophy) (see DiCarlo, J. E. et al. Genome engineering in Saccharomyces cerevisiae using CRISPR-Cas systems, Nucleic Acids Res. 41, 4336-4343 (2013)) (Addgene #43802). One or the other was used in each mating experiment. The pRS413 vector was transformed into select cell types to confer histidine prototrophy as a marker to select for diploid cells. Strain genotypes are provided in Table 1 below.

TABLE 1

| Strain | Genotype |
|---|---|
| SK1 A | MATa ho::LYS2 lys2 ura3 leu2::hisG his3::hisG trp1::hisG |
| SK1 α | MATα ho::LYS2 lys2 ura3 leu2::hisG his3::hisG trp1::hisG |
| Y12A | MATa ho::HygMX ura3::KanMX |
| YPS128 | MATa ho::HygMX ura3::KanMX |
| YJM981 | MATa ho::HygMX ura3::KanMX |
| Y55 | MATa ho::HygMX ura3::KanMX |
| UWOPS05- | MATa ho::HygMX ura3::KanMX |
| 217.3 | |
| DBVPG | MATa ho::HygMX ura3::KanMX |
| 6044 | |
| 273614N | MATa ho::HygMX ura3::KanMX |

### Example IX

### Yeast Mating Experiments

Haploid drive-containing SK1 yeast and haploid wild-type strains of the opposite mating type were mixed in equal amounts in YPAD liquid media and incubated overnight. The resulting diploids were washed in sterile water and plated on selective media for both parental genotypes. Table 2 below details the specific crosses.

TABLE 2

| MATa Genotype | MATα Genotype | Selection |
|---|---|---|
| SK1 pRS414-Cas9 | SK1 ade2::gRNA (gene drive), pRS413 | SC-histidine - tryptophan |
| SK1 ade2::gRNA + | SK1 pRS413 | SC-histidine - tryptophan |
| URA3 (gene drive), | | |
| p414-Cas9 | | |
| SK1 p414-Cas9 | SK1 abd1::ABD1 recoded + gRNA (gene | SC-histidine - tryptophan |
| | drive), pRS413 | |
| Y12A Hygromycin | SK1 ade2::gRNA (gene drive), p416-Cas9 | SC-uracil + 300 ug/mL |
| B resistance (HygR) | | Hygromycin B |
| YPS128 | SK1 ade2::gRNA (gene drive), p416-Cas9 | SC-uracil + 300 ug/mL |
| Hygromycin B | | Hygromycin B |
| resistance (HygR) | | |
| YJM981 | SK1 ade2::gRNA (gene drive), p416-Cas9 | SC-uracil + 300 ug/mL |
| Hygromycin B | | Hygromycin B |
| resistance (HygR) | | |
| Y55 Hygromycin B | SK1 ade2::gRNA (gene drive), p416-Cas9 | SC-uracil + 300 ug/mL |
| resistance (HygR) | | Hygromycin B |
| UWOPS05-217.3 | SK1 ade2::gRNA (gene drive), p416-Cas9 | SC-uracil + 300 ug/mL |
| Hygromycin B | | Hygromycin B |
| resistance (HygR) | | |
| DBVPG 6044 | SK1 ade2::gRNA (gene drive), p416-Cas9 | SC-uracil + 300 ug/mL |
| Hygromycin B | | Hygromycin B |
| resistance (HygR) | | |
| 273614N | SK1 ade2::gRNA (gene drive), p416-Cas9 | SC-uracil + 300 ug/mL |
| Hygromycin B | | Hygromycin B |
| resistance (HygR) | | |
| SK1 ADE2::ADE2 | SK1 ade2::gRNA (gene drive), pRS413 | SC-histidine - tryptophan |
| silently recoded | | |
| genomic target seed | | |
| sequence, p414- | | |
| Cas9 | | |
| SK1, p414-empty | SK1 ade2::gRNA (gene drive), pRS413 | SC-histidine - tryptophan |

### Example X

### Sporulation and Tetrad Dissection

After mating in liquid YPAD and selection for diploids on selection plates, the selection plates were scraped into 10 mL selective media and grown overnight at 30° C. A fresh 5 mL YPAD culture was then inoculated to and OD=0.1 and grown 4-5 hours at 30° C. The entire culture was then washed twice in 10 mL water, inoculated into 2 mL of sporulation media (1% potassium acetate), and incubated at room-temperature for 3 days or until spores were visible. Sporulated cells were suspended in 50 μL of a stock solution of zymolyase (50 μg/mL in 1M sorbitol) and incubated at 30C for 5 minutes, transferred to ice, diluted with 150 μL cold H₂O, microdissected using a Zeiss tetrad dissection microscope, and isolated spores grown on YPAD plates.

### Example XI

### Selection for URA3 Function

Dissected spores were grown in synthetic complete (SC) media and then spotted onto SC medium as well as SC medium without uracil. To enhance red color, all SC solid media used for plate images contained 0.5 X adenine hemisulfate (final concentration of 0.08 mM).

### Example XII

### Quantitative PCR

Candidate primer pairs were designed to amplify short regions specific to each drive or the wild-type sequence replaced by the drive, as well as the ACT1 gene as a control. All sequences are included in the supplementary information. Genomic DNA was extracted using Method A as described in Looke et al. 31

KAPA SYBR FAST qPCR Master Mix (2X) was used to perform the qPCR reaction along with 25 ng of genomic DNA. The amplification efficiency and relative specificity of each primer pair were measured by amplifying dilutions of genomic DNA from wild-type and drive haploids, respectively, and the best-performing and well-matched pairs selected for use (see below for all primers used). Quantitative PCR reactions were performed on genomic DNA isolated from each parental haploid as well as from diploids arising from three independent mating events. Three reactions (technical replicates) were performed per sample on a LightCycler 96 machine by Roche.

### Example XIII

### Calculations

Results from three technical replicates were averaged for calculations. In order to directly calculate the ratio of alleles before PCR amplification, the efficiencies of the different primer pairs were first determined. Efficiencies were calculated from qPCR runs of serial dilutions (6 orders of magnitude) as:

Efficiency=10^{-1/slope}

R² values were higher than 0.99 in all cases except for one pair (ade2::URA3+sgRNA).
The allelic ratios were calculated as:

xₐ·Eₐ^{Ct,a}=x_{b}·E_{b}^{Ct,b}

xₐ/x_{b}=E_{b}^{Ct,b}/Eₐ^{Ct,a}

with xₐ and x_{b} being the initial concentration of drive and wt DNA,

Eₐ and E_{b} the efficiency of the respective primer pairs and

Ct,a and Ct,b the Ct values for each sample.

### Example XIV

### Efficiency of CRISPR/Cas9 Gene Drives in Yeast

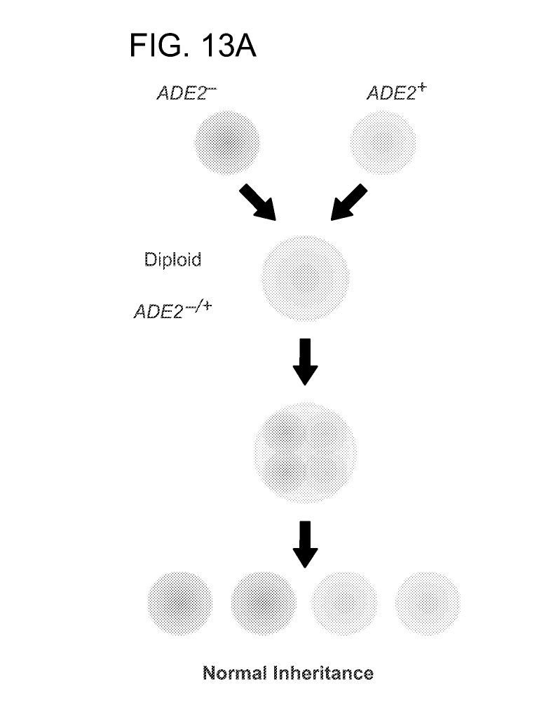

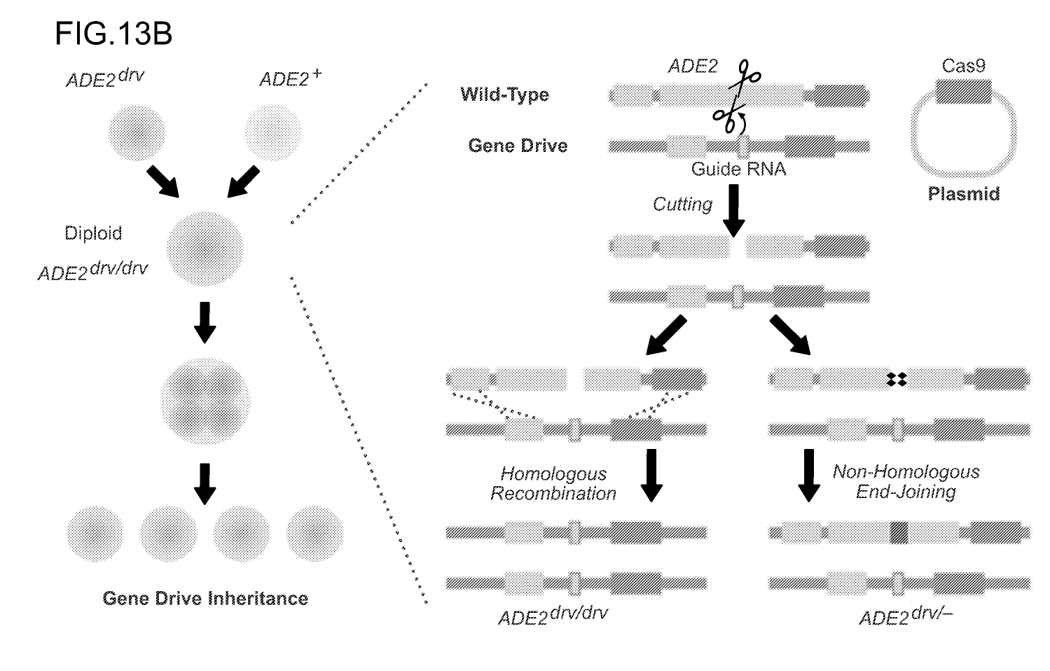

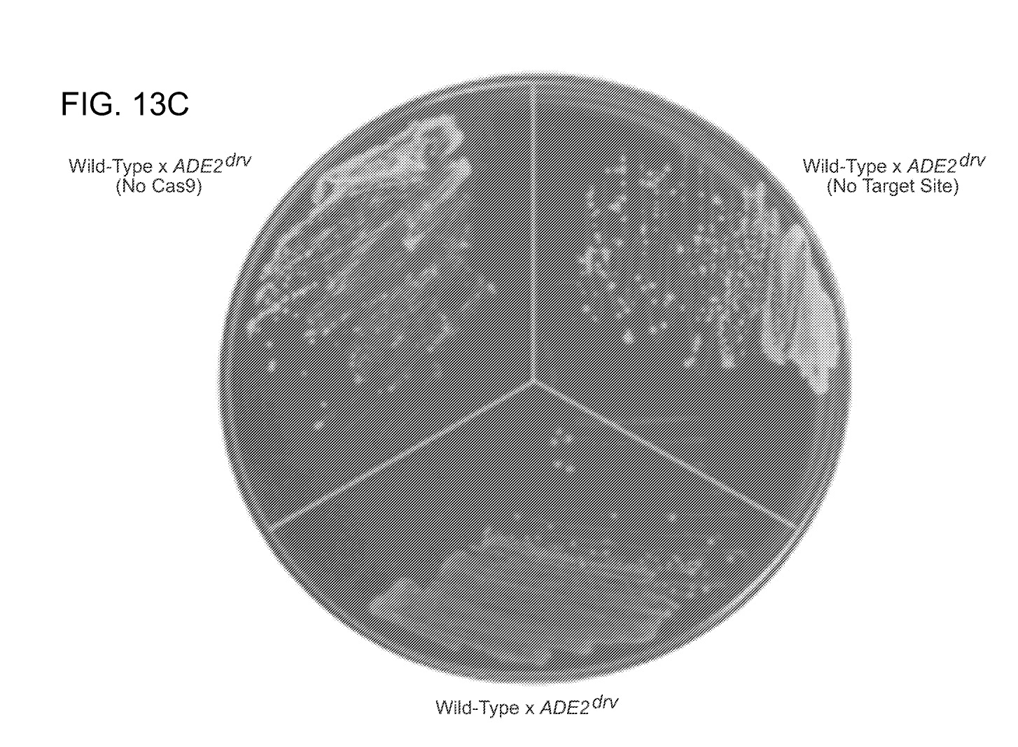

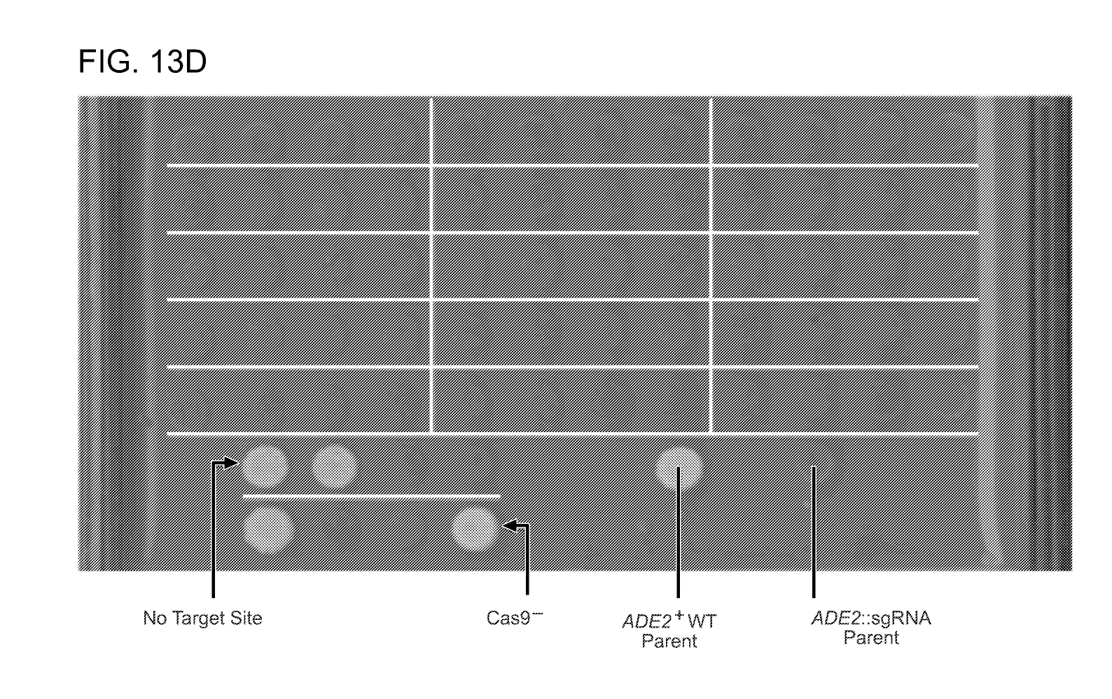

To directly measure the efficiency of CRISPR/Cas9 gene drives in yeast, a system was developed using the red color that builds up in yeast lacking functional copies of the ADE2 gene. See Chamberlain, N., Cutts, N. S. & Rainbow, C. The formation of pigment and arylamine by yeasts. J. Gen. Microbiol. 7, 54-60 (1952). As depicted in FIG. 13A, if red ADE2⁻ haploids are mated with cream-colored wild-type haploids, the resulting heterozygous diploids inherit one functional copy and are therefore cream-colored. When these diploids undergo meiosis and reproduce via sporulation, half of the resulting haploids inherit the broken copy and are consequently red; the other half inherit the intact copy and are cream-colored (FIG. 13A).

As depicted in FIG. 13B, if the red haploids encode a functional gene drive that cuts and replaces the intact ADE2 locus inherited from the wild-type parent, their diploid progeny will be red. Because these red diploids will have two broken copies of ADE2, all of their sporulated haploid offspring will inherit a broken copy and consequently will also be red. Thus, the cutting efficiency of a gene drive that targets and replaces ADE2 can be assessed by simply plating diploids and counting the fraction that are red.

A gene drive construct targeting ADE2 was made. To prevent accidental escape of the gene drive into the wild, Cas9 and guide RNAs were separated to avoid creating a self-sufficient inheritance-biasing cassette. Consequently, constructs encoded a guide RNA targeting ADE2, while Cas9 was provided from an episomal plasmid. Red haploids were mated to wild-type yeast of the opposite mating type in the presence or absence of the plasmid and plated on media that selects for diploids. As shown in FIG. 13C, nearly all colonies were red in the presence of the plasmid, indicating highly efficient cutting of the ADE2 copy inherited from the wild-type parent. In the absence of Cas9, no red diploid colonies were observed, demonstrating that the drive can only spread in laboratory yeast populations that provide it.

To verify that the ADE2 alleles from wild-type parents were lost, the mated diploids were sporulated and their resultant haploid progeny were examined. As shown in FIG. 13D, upon dissecting 18 cas9+ diploids, a perfect 4:0 ratio of red:cream haploids was observed, confirming that all copies of the ADE2 locus were disrupted. In contrast, 18 cream-colored cas9—diploids yielded a 2:2 red:cream ratio, indicating normal inheritance of the inactivated drive and the wild-type alleles.

To determine whether the ADE2 disruptions in red diploids were the result of successful copying of the drive element by homologous recombination, 72 haploids derived from dissected cas9+ diploids were sequenced. All sequenced colonies contained intact drives without additional mutations, indicating that drive mobilization was efficient and occurred at high fidelity.

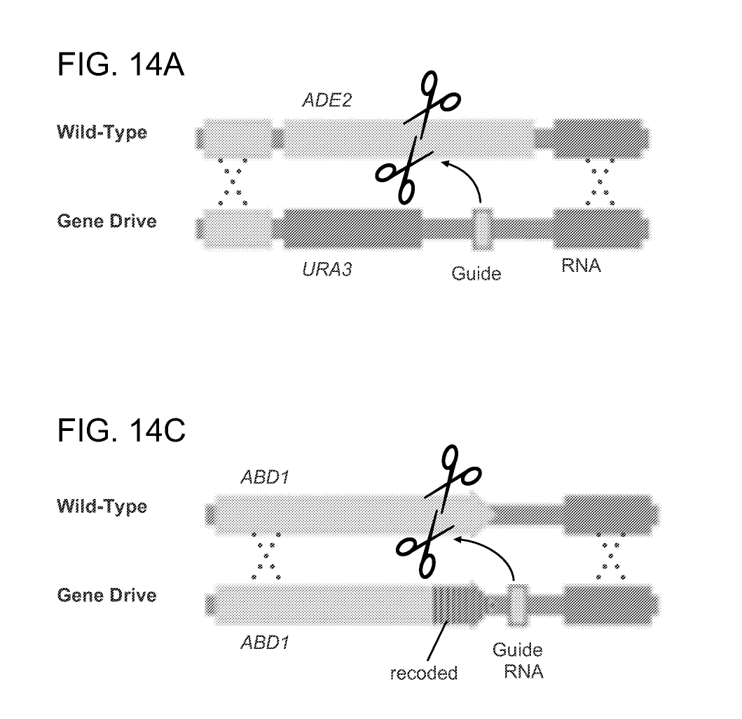

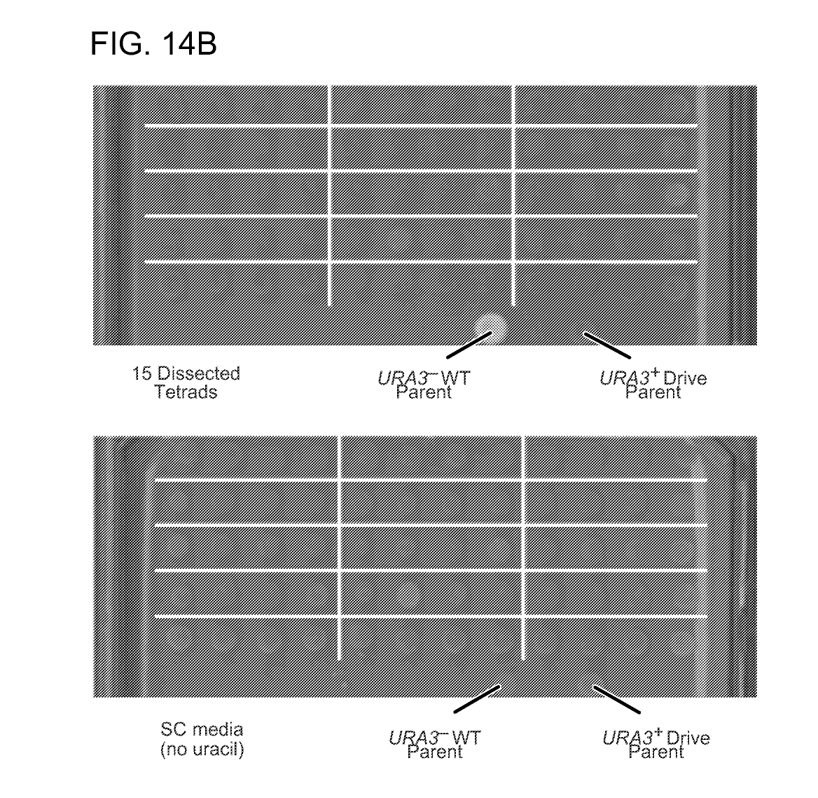

An ADE2 gene drive was modified as shown in schematic in FIG. 14A to contain an in cis URA3 allele, which allows laboratory modified yeast strains to grow in the absence of uracil supplementation. This drive element was tested for the ability of gene drives to “carry” an associated cargo element when copied into a target locus. URA3-containing drive haploids were mated with wild-type haploids in the presence of an episomal Cas9 plasmid, diploids were selected (all of which were red) and sporulated, and 18 tetrads were dissected. As was the case for the original ADE2 gene drive, all of the sporulated haploid cells formed red colonies. As shown in FIG. 14B, all grew normally when replica plated on uracil deficient media, indicating that the URA3 cargo element was efficiently copied with the drive.

Gene drives which target and recode an essential gene could avoid drive resistance in even large populations since error prone repair events that modify the target site will cause lethality. Non-essential but nonetheless important genes could similarly be edited because mutants created by NHEJ events would still be less fit than the drive itself. To test essential gene recoding during drive insertion, a third gene drive targeting ABD1 shown in schematic in FIG. 14C was constructed. See Mao, X., Schwer, B. & Shuman, S. Mutational analysis of the Saccharomyces cerevisiae ABD1 gene: cap methyltransferase activity is essential for cell growth, Mol. Cell. Biol. 16, 475-480 (1996).

A haploid strain containing a recoded ABD1 allele upstream of a guide RNA targeting the natural ABD1 coding sequence was mated to wild-type cells in the presence of Cas9. Diploid cells were selected of which 18 were sporulated 18 of them. 72 segregants were sequenced. All contained the recoded ABD1 locus and the guide RNAs, thereby demonstrating gene drives based on essential gene recoding.

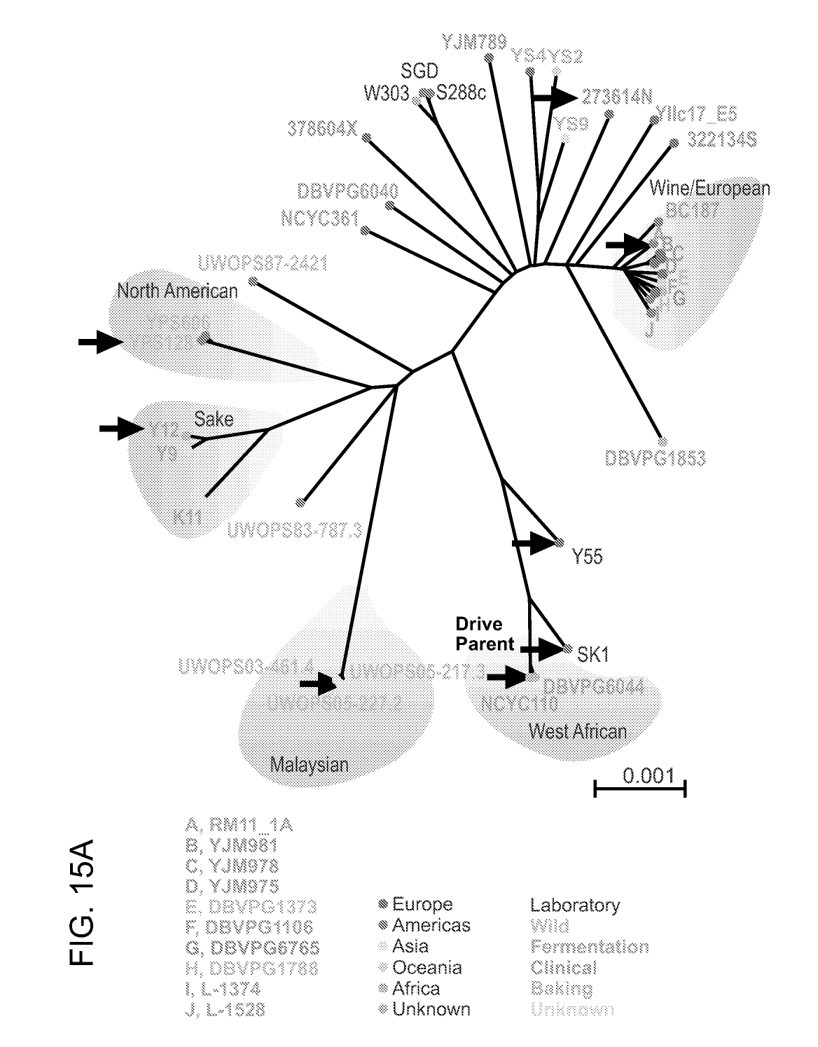

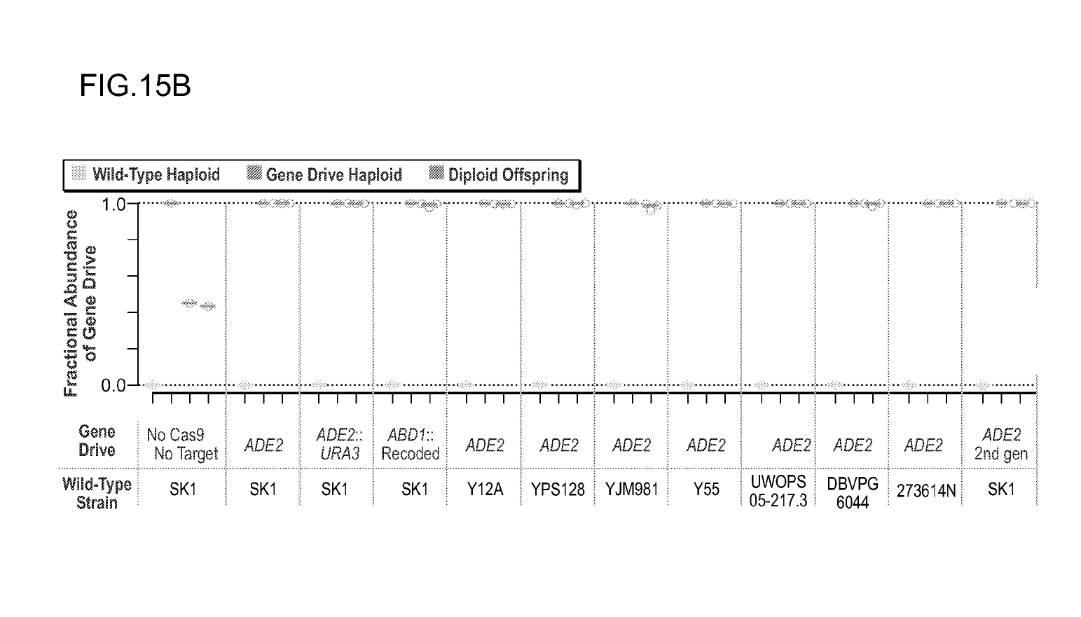

Gene drives were copied from a laboratory strain into a diverse group of native S. cerevisiae strains. ADE2 drive-containing haploids were mated with 6 phylogenetically and phenotypically diverse wild-type strains of haploid S. cerevisiae. See Liti, G. et al. Population genomics of domestic and wild yeasts, Nature 458, 337-341 (2009). See also FIG. 15A. To quantitatively measure the efficiency of gene drive copying for each cross, quantitative PCR was performed on populations of all diploids using one set of primers specific to the drive and another set designed to amplify either wild-type or NHEJ-disrupted alleles. As shown In FIG. 15B which was generated using BoxPlot (see Spitzer, M., Wildenhain, J., Rappsilber, J. & Tyers, M. BoxPlotR: a web tool for generation of box plots. Nat. Methods 11, 121-122 (2014)), the fraction of diploid chromosomes containing the ADE2 gene drive was over 99% regardless of wild-type parent, demonstrating the use of the drive in diverse backgrounds. Addition of the URA3 cargo gene did not appreciably change this efficiency. The ABD1 drive was copied at an equivalent rate.

The stability of the drive over successive copying events was investigated. As shown in FIG. 15B, several haploid offspring of the first-round ADE2 gene drive diploids were mated to wild-type haploids containing the Cas9-expressing plasmid. All of the second-generation gene drive constructs biased inheritance at the same efficiency, demonstrating a continued ability to spread through sexually reproducing populations over generations.

The following genome modification primer and gBlock sequences are provided.

| | Sequence |
|---|---|
| Genome modification | |
| primers and gBlocks | |
| ADE2.sgRNA.ade2.1 | TACGAACCGGGTAATACTAAGTGATTGACTCTTGCTGACCT |
| insert.F | TTTATTAAGAACTAAATGGtctttgaaaagataatgtatgattatgctttc |
| | (SEQ ID NO: 5) |
| ADE2.sgRNA.ade2.1 | TAATAAGTGATCTTATGTATGAAATTCTTAAAAAAGGACACC |
| insert.R | TGTAAGCGTTGATTTCTAagacataaaaaacaaaaaaagcaccac |
| | (SEQ ID NO: 6) |
| gRNA + | TACGAACCGGGTAATACTAAGTGATTGACTCTTGCTGACCT |
| CaURA3.ade2.F | TTTATTAAGAACTAAATGGagacataaaaaacaaaaaaagcaccaccg |
| | (SEQ ID NO: 7) |
| gRNA + | TAATAAGTGATCTTATGTATGAAATTCTTAAAAAAGGACACC |
| CaURA3.ade2.R | TGTAAGCGTTGATTTCTAtcgacactggatggcggcgttagtatc |
| | (SEQ ID NO: 8) |
| ABD1.recode + gRNA | AGCCAGATGCCATTCAACAAGTTCTTCGTGCAGGAGATACC |
| | AAAGTGGATAGAACGTTTCAGCCCAAAGATGCGTGAGGGG |
| | CTTCAGCGTAGCGACGGGCGTTACGGGGTGGAGGGTGACG |
| | AGAAAGAGGCTGCTAGCTACTTTTACACGATGTTCGCTTTT |
| | AGAAAAGTTAAGCAATACATAGAGCCTGAGTCAGTTAAACC |
| | AAATTGAACGGCTCCTCGCTGCAGACCTGCGAGCAGGGAA |
| | ACGCTCCCCTCACAGACGCGTTGAATTGTCCCCACGCCGCG |
| | CCCCTGTAGAGAAATATAAAAGGTTAGGATTTGCCACTGAG |
| | GTTCTTCTTTCATATACTTCCTTTTAAAATCTTGCTAGGATAC |
| | AGTTCTCACATCACATCCGAACATAAACAACCATGGGTATG |
| | ACCGACCAAGCGACGCCCAACCTGCCATCACGAGATTTCG |
| | ATCCCACCGCCGCCTTCTATGAAAGGtctttgaaaagataatgtatgattat |
| | gctttcactcatatttatacagaaacttgatgttttctttcgagtatatacaaggtgattacatgtacgttt |
| | gaagtacaactctagattttgtagtgccctcttgggctagcggtaaaggtgcgcattttttcacaccc |
| | tacaatgttctgttcaaaagattttggtcaaacgctgtagaagtgaaagttggtgcgcatgtttcggc |
| | gttcgaaacttctccgcagtgaaagataaatgatcTGAAGGGGATGAAAAGGA |
| | AGGTTTTAGAGCTAGAAATAGCAAGTTAAAATAAGGCTAGT |
| | CCGTTATCAACTTGAAAAAGTGGCACCGAGTCGGTGGTGC |
| | TTTTTTTGTTTTTTATGTCT |
| | (SEQ ID NO: 9) |
| ABD1.recode + | tatgttgtgccattcgaaaccttaagaagtttggctgatgaatacggtttggaactagtaAGCCA |
| gRNA.int.F | GATGCCATTCAACAAGTTC |
| | (SEQ ID NO: 10) |
| ABD1.recode + | gtaatacggccgaaatacagatgctttatagtagggttattgtttctattcatttttattAGACATA |
| gRNA.int.R | AAAAACAAAAAAAGCACCACC |
| | (SEQ ID NO: 11) |
| ADE2.gRNA | Tctttgaaaagataatgtatgattatgctttcactcatatttatacagaaacttgatgttttctttcgagta |
| | tatacaaggtgattacatgtacgtttgaagtacaactctagattttgtagtgccctcttgggctagcgg |
| | taaaggtgcgcattttttcacaccctacaatgttctgttcaaaagattttggtcaaacgctgtagaagt |
| | gaaagttggtgcgcatgtttcggcgttcgaaacttctccgcagtgaaagataaatgatcACTTG |
| | AAGATTCTTTAGTGTGTTTTAGAGCTAGAAATAGCAAGTTA |
| | AAATAAGGCTAGTCCGTTATCAACTTGAAAAAGTGGCACCG |
| | AGTCGGTGGTGCTTTTTTTGTTTTTTATGTCT |
| | (SEQ ID NO: 12) |
| ADE2.1.silent. | TGATGTGCTAACGATTGAGATTGAGCATGTTGATGTTCCTAC |
| seed.90mer.F | CCTGAAAAACCTGCAAGTAAAACATCCCAAATTAAAAATTT |
| | ACCCTTC |
| | (SEQ ID NO: 13) |
| ADE2.1.silent. | GAAGGGTAAATTTTTAATTTGGGATGTTTTACTTGCAGGTTT |
| seed.90mer.F | TTCAGGGTAGGAACATCAACATGCTCAATCTCAATCGTTAG |
| | CACATCA |
| | (SEQ ID NO: 14) |
| ABD1.ver.F | ATAGATAATGTTCCTGAATATGTTGTGCCA |
| | (SEQ ID NO: 15) |
| ABD1.ver.R | TTACTACATATAGAAGTCTTGTAATACGGCCG |
| | (SEQ ID NO: 16) |
| ADE2.ver.F | GCTACGAACCGGGTAATACTAAGTGATTG |
| | (SEQ ID NO: 17) |
| ADE2.ver.R | CAGGTAATTATTCCTTGCTTCTTGTTACTGG |
| | (SEQ ID NO: 18) |
| qPCR Primers | |
| ade2.WT.qPCR.F | TACGAACCGGGTAATACTAAGTGATTGACTC |
| | (SEQ ID NO: 19) |
| ade2.gRNA.qPCR.R | CGCTAGCCCAAGAGGGCACTACA |
| | (SEQ ID NO: 20) |
| ade2.WT.qPCR.R | TACCAACTGTTCTAGAATCCATACTTGATTGTTT |
| | (SEQ ID NO: 21) |
| URA3.genedrive. | TACGAACCGGGTAATACTAAGTGATTGACTC |
| ade2.WT.qPCR.F | (SEQ ID NO: 22) |
| URA3.genedrive. | CCTCCTAATATACCAACTGTTCTAGAATCCAT |
| ade2.WT.qPCR.R | (SEQ ID NO: 23) |
| URA3.genedrive. | AAACTTCTCCGCAGTGAAAGATAAATGATC |
| ade2.gRNA.qPCR.R | (SEQ ID NO: 24) |
| ABD1_rec_qPCR.R | CGAGGAGCCGTTCAATTTGGTTTAACTGAC |
| | (SEQ ID NO: 25) |
| ABD1_rec_qPCR.F | AGATGCGTGAGGGGCTTCAGC |
| | (SEQ ID NO: 26) |
| ABD1_WT_qPCR | GAAGGGGATGAAAAGGAAGC |
| (JDwt1.4).F | (SEQ ID NO: 27) |
| ABD1_WT_qPCR | CGCTTTCCGGTTCGATATAC |
| (JDwt1.3).R | (SEQ ID NO: 28) |
| ACT1.qPCR.F | CGAAAGATTCAGAGCCCCAGAAGCT |
| | (SEQ ID NO: 29) |
| ACT1.qPCR.R | CGGTGATTTCCTTTTGCATTCTTTCG |
| | (SEQ ID NO: 30) |

### Example XIV

### Dependent Gene Drives

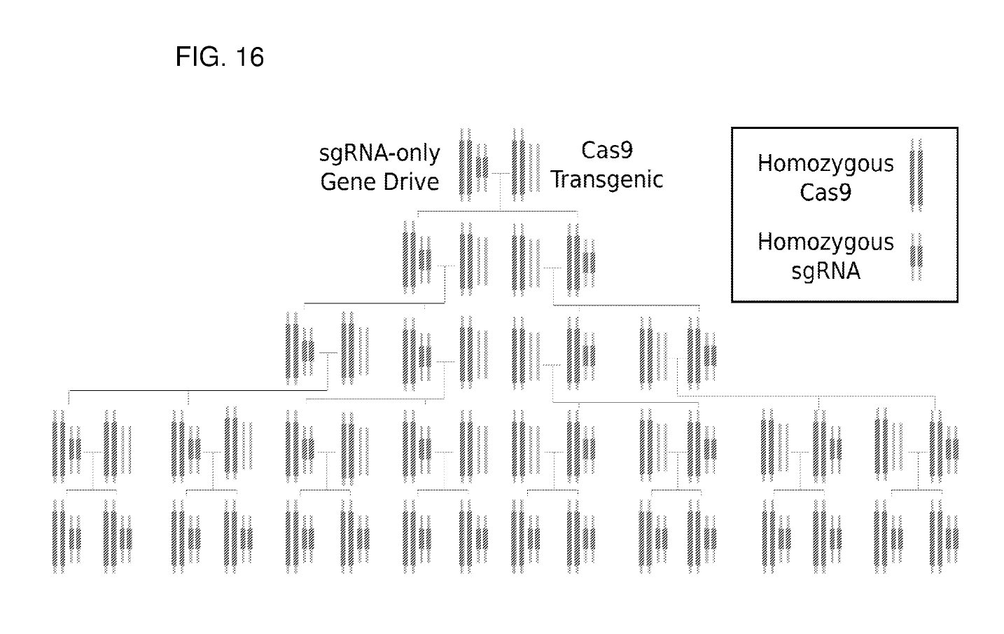

Aspects of the present disclosure are directed to gene drive designs in a cell or organism that are capable of biasing inheritance in its favor for only a limited number of generations, thereby enabling the release of small number of organisms to exclusively alter a specified fraction of the local population into which the organism is released. According to one aspect, separating the components of a CRISPR-based gene drive element such that one component does not exhibit drive (referred to herein as a “split drive’) is utilized as a method of preventing the driving element from altering wild populations in the event of drive-carrying organisms accidentally escaping the lab. One such arrangement involves a gene drive that encodes one or more guide RNAs but not the Cas9 gene. The gene drive that encodes the one or more guide RNAs is referred to as the dependent ‘split drive’ element (referred to herein as element Z), which will consequently exhibit drive only when present in an organism encoding Cas9 elsewhere in the genome. The Cas 9 component is referred to as the non-driving required component (referred to herein as element Y). That is, a classical split drive used for confinement can be annotated as Y->Z, wherein non-driving component Y that is expressed in the cell drives split drive component Z whenever Y is present so that component Z can insert itself into the genome making the cell homozygous for element Z. It is to be understood that the dependent gene drive systems described herein can be designed and utilized for any of the preceding uses and methods described herein, as though set forth directly herein. Though useful for confinement purposes, such a system is predicted to spread to fixation in a population in which all organisms encode the non-driving component (FIG. 16) much like an autonomous RNA-guided gene drive does in a susceptible population. FIG. 16 depicts inheritance of a split RNA-guided gene drive element in a population encoding the required non-driving element. FIG. 16 shows a family tree of a split RNA-guided gene drive element (where green, represents the sgRNA) in a population where all individuals encode the required non-driving element (where brown represents the RNA-guided nuclease such as Cas9), assuming each generation exclusively mates with individuals lacking the driving element. The split drive element is predicted to sweep to fixation absent interfering factors as would an autonomous gene drive in a susceptible population.

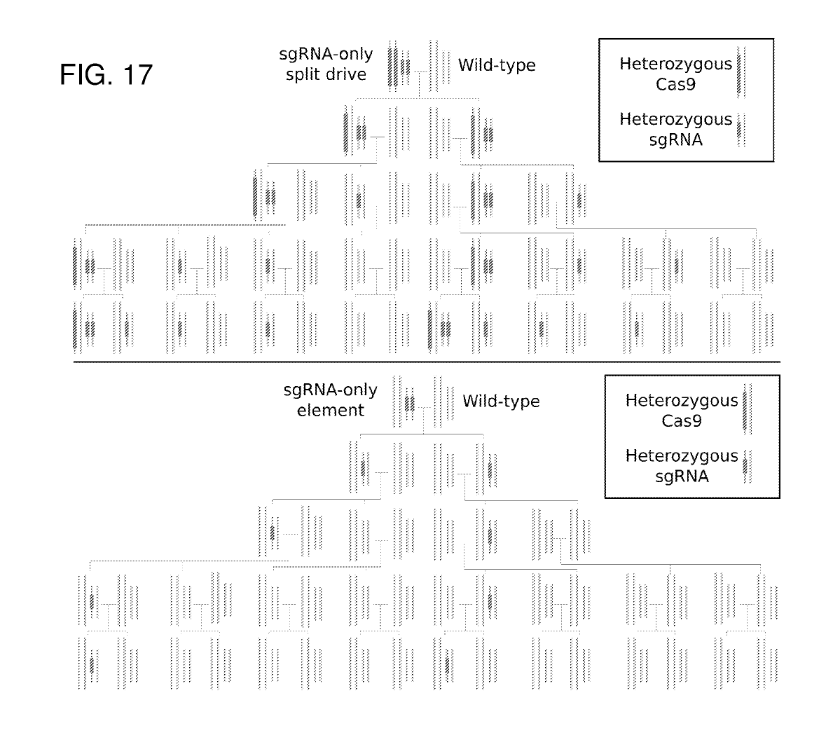

As depicted in FIG. 17, a split drive will increase in abundance due to inheritance distortion in populations lacking the non-driving component (e.g. in the event of organismal escape) as compared to inheritance of a normal gene. The top of FIG. 17 depicts a family tree of a split RNA-guided gene drive element (where green represents sgRNA) in a population where no individuals encode the required non-driving element (e.g. an RNA-guided nuclease), assuming each generation exclusively mates with individuals lacking the driving element. The incidence of the split drive element increases substantially more than does an equivalent non-driving element (as shown in the bottom of FIG. 17). As shown in FIG. 17, Z will increase in abundance in a manner that is linearly dependent on the incidence of Y. So long as the escaped organisms do not constitute a substantial fraction of the wild population, the incidence of Y is negligible relative to the total population and the increase in Z correspondingly negligible, validating the split drive design for the purposes of confinement.

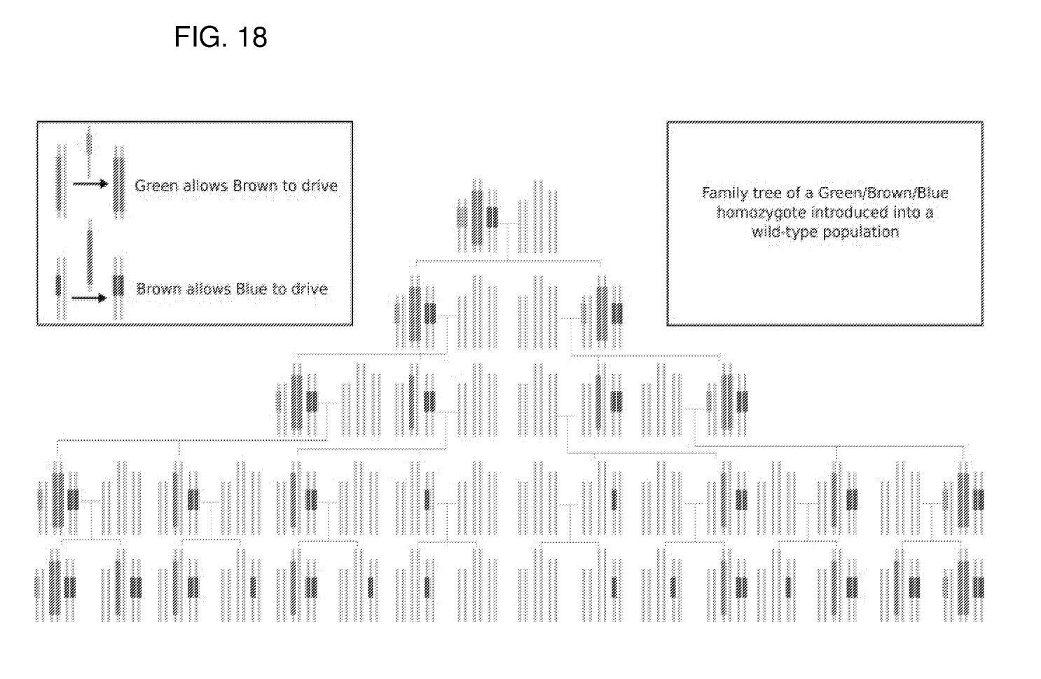

Since Z will linearly increase in a Y-dependent manner, the increase in Z is substantially accelerated by making Y increase linearly, thereby multiplying the factors of increase. That is, aspects described herein add another link in the split drive chain such that X->Y->Z, i.e. component Z depends on component Y which depends on component X. This arrangement of dependency is referred to herein as a “daisy chain gene drive.” In this case a comparatively small release of organisms encoding this arrangement will result in a fixed amount of X, a linearly increasing Y, and a more rapidly increasing Z as shown in FIG. 18 which depicts an inheritance family tree of a daisy chain gene drive A-B-C assuming each generation exclusively mates with wild-type individuals. According to additional aspects, a “daisy chain” gene drive model can be extended indefinitely to create (W->X->Y->Z), i.e. component Z depends on component Y which depends on component X which depends on component W or (V->W->X->Y->Z), i.e. component Z depends on component Y which depends on component X which depends on component W which depends on component V and so on. Further expansion of the chain exponentially accelerates the spread of Z.

The present disclosure provides a method of building a daisy chain of mutually dependent elements required to exhibit RNA-guided gene drive (referred to as a “dependent gene drive”) that will bias inheritance for a fixed number of generations, thereby permitting the alteration of any desired fraction of any population susceptible to an RNA-guided gene drive. Representative embodiments of dependent gene drive designs are depicted in FIG. 19. In such configurations, the dependent drives enable an element Z to be driven to high incidence or even fixation in local populations by releasing a relatively small number of organisms without the risk of causing extensive spread through more distant populations. Various dependent gene drive X->Y->Z daisy chain drive architectures are shown compared to an autonomous gene drive. In all cases, the objective is to drive the desired gene (dark green) through the population by cutting locus Z. Variants 1 through 3 depict different arrangements of RNA-guided nuclease (such as Cas9 represented by brown) and guide gRNAs represented by green directing the nuclease to cut the different loci. The nuclease may optionally be encoded at additional loci for redundancy in any of the versions. In these representative examples, each element (when viewed from right to left) requires the previous element in the chain if it is to exhibit drive, save for the last element (far left)—in this case the one in locus X—which cannot drive under any circumstances. In contrast, any element encoding all the factors necessary to cut and drive through its own locus is a fully autonomous gene drive expected to spread indefinitely through the population (bottom). Variant 3 is a split drive element inserted into element Z that requires Cas9 supplied from a different locus (Y) to drive but has been further converted into a daisy chain drive by inserting guide RNAs targeting locus Y into locus X (far left).

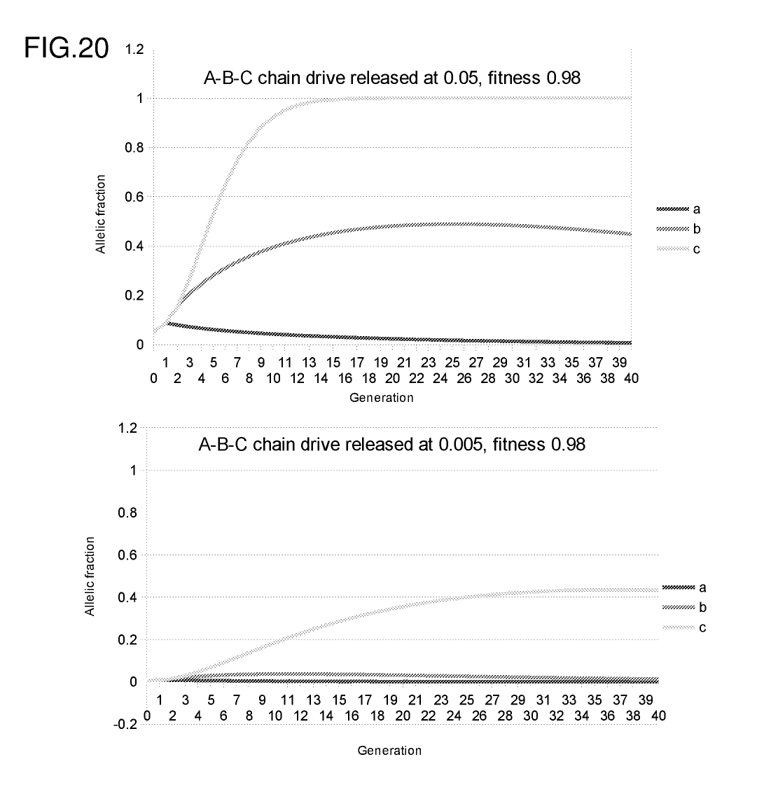

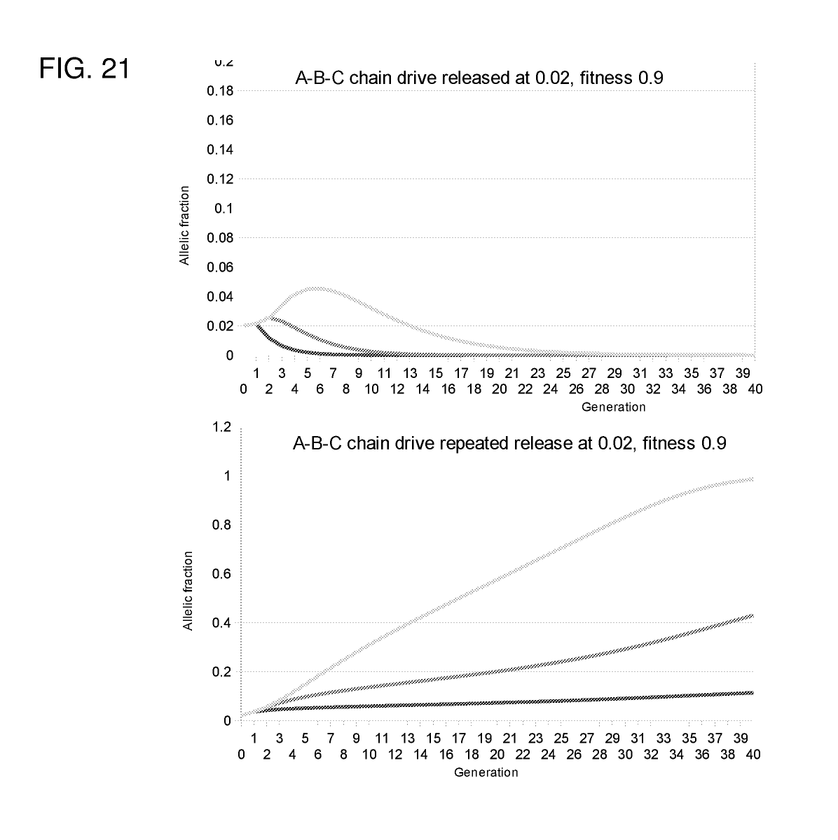

An analytical model of allele frequencies in a local panmictic population upon the release of a chain drive (X->Y->Z) at an arbitrary frequency into that population was constructed given certain assumptions concerning the frequency at which the drive is copied and other relevant parameters such as drive fitness. The model clearly demonstrates that the initial release of a small number of organisms can alter a large fraction of the local population for a variety of fitness costs. If desired, this can lead to fixation of the Z allele (FIG. 20, top) or cause the frequency of Z to plateau at a desired level before slowly declining due to the fitness cost (FIG. 20, bottom). Repeated releases of organisms carrying a daisy drive can cause the Z allele to reach fixation when a single release would not (FIG. 21). Controlling the numbers released over time can consequently maintain the allele within a given frequency range as needed.

Aspects of the present disclosure are directed to a method of altering a eukaryotic germline cell of an organism including inserting into the first and second chromosomes of a chromosome pair a first foreign nucleic acid sequence including a cargo desired gene and including flanking sequences corresponding to a first target locus, wherein the cargo desired gene is to be inserted into the genome of progeny of the germline cell by an RNA guided DNA binding protein nuclease system, inserting into the first and second chromosomes of a chromosome pair a second foreign nucleic acid sequence encoding an RNA guided DNA binding protein nuclease and one or more guide RNAs, and including corresponding promoter sequences,

wherein the sites of insertion of the first foreign nucleic acid sequence and the second foreign nucleic acid are separated by at least 1000, 2000, 5000, 10000, 100000, 1000000 bases if on the same chromosome pair, wherein the one or more guide RNAs encoded by the second foreign nucleic acid sequence are complementary to one or more target locations on genomic DNA at the first target locus which is an insertion site for the first foreign nucleic acid sequence. According to one aspect, the RNA guided DNA binding protein nuclease is Cas9. According to one aspect, the RNA guided DNA binding protein nuclease is a Cas9 nickase. According to one aspect, at least one target location is within an essential gene and the foreign nucleic acid sequence is inserted adjacent to and replacing part of the essential gene. According to one aspect, the first foreign nucleic acid sequence is inserted by homologous recombination. According to one aspect, the germline cell is a fungal cell, a plant cell, an insect cell or a mammalian cell.

Aspects of the present disclosure are directed to a method of altering a eukaryotic germline cell including (1) inserting into a first and second chromosome a first nucleic acid encoding a guide RNA complementary to a target location on a target locus Y and including flanking regions identical to regions on a target locus X, (2) inserting into a first and second chromosome a second nucleic acid sequence encoding a guide RNA complementary to a target location on a target locus Z and/or a Cas9 protein and including flanking regions identical to regions on a target locus Y, and (3) inserting into a first and second chromosome a third nucleic acid sequence encoding a desired cargo gene and including flanking regions identical to regions on a target locus Z, wherein the third nucleic acid sequence encodes Cas9 when the second nucleic acid sequence encodes a guide RNA, wherein the third nucleic acid sequence lacks either Cas9 or a guide RNA when the second nuclease acid sequence encodes both Cas9 and a guide RNA, or wherein the third nucleic acid sequence encodes a guide RNA when the second nucleic acid sequence encodes Cas9, wherein the first nucleic acid sequence, the second nucleic acid sequence or the third nucleic acid sequence are on the same or different chromosomes, wherein the first nucleic acid sequence, the second nucleic acid sequence and/or the third nucleic acid sequence are separated by at least 1000, 2000, 5000, 10000, 100000, 1000000 bases if on the same chromosome pair. According to one aspect, the first nucleic acid sequence, the second nucleic acid sequence and the third nucleic acid sequence include one or more corresponding promoter sequences. According to one aspect, the Cas9 protein is an enzymatically active Cas9. According to one aspect, the Cas9 protein is a Cas9 nuclease or a Cas9 nickase. According to one aspect, the target locus Z is within an essential gene and the desired cargo gene is inserted adjacent to and replacing part of the essential gene. According to one aspect, the third nucleic acid sequence is inserted by homologous recombination. According to one aspect, the germline cell is a fungal cell, a plant cell, an insect cell or a mammalian cell. According to one aspect, inheritance of a genome editing system having mutually dependent components is limited to a desired fixed number of generations of the target organism.

Aspects of the present disclosure are directed to a method of altering a eukaryotic germline cell including (1) inserting into a first and second chromosome of a first chromosome pair a first nucleic acid encoding a desired cargo gene for insertion into a first target locus and including flanking regions identical to regions on the first target locus, wherein the first nucleic acid sequence optionally includes a Cas9 protein or a guide RNA complementary to the first target locus, (2) inserting into a first and second chromosome of a second chromosome pair a second nucleic acid sequence encoding a guide RNA complementary to the first target locus and/or a Cas9 protein and including flanking regions identical to regions on a second target locus, and (3) inserting into a first and second chromosome of a third chromosome pair a third nucleic acid sequence encoding a guide RNA complementary to the second target locus and/or a Cas9 protein and including flanking regions identical to regions on the second target locus, and (4) inserting into a first and second chromosome of an Nth chromosome pair an Nth nucleic acid sequence encoding a guide RNA and optionally a Cas9 protein and including corresponding promoters, wherein the guide RNA is complementary to a target locus on genomic DNA at an insertion site of an Nth-1 nucleic acid sequence encoding a guide RNA and optionally a Cas9 protein, wherein the first nucleic acid sequence, the second nucleic acid sequence, the third nucleic acid sequence or the Nth nucleic acid sequence are on the same or different chromosomes,

wherein the first nucleic acid sequence, the second nucleic acid sequence, the third nucleic acid sequence or the Nth nucleic acid sequence are separated by at least 1000, 2000, 5000, 10000, 100000, 1000000 bases if on the same chromosome pair. According to one aspect, the first nucleic acid sequence, the second nucleic acid sequence, the third nucleic acid sequence or the Nth nucleic acid sequence include one or more corresponding promoter sequences. According to one aspect, the Cas9 protein is an enzymatically active Cas9. According to one aspect, the Cas9 protein is a Cas9 nuclease or a Cas9 nickase. According to one aspect, the target locus Z is within an essential gene and the desired cargo gene is inserted adjacent to and replacing part of the essential gene. According to one aspect, the third nucleic acid sequence is inserted by homologous recombination. According to one aspect, the germline cell is a fungal cell, a plant cell, an insect cell or a mammalian cell. According to one aspect, the method includes further including the step of growing the germline cell into the organism. According to one aspect, the method further includes the step of growing the germline cell into the organism and mating the organism with a wild-type organism of the same species to produce an offspring germline cell including a first chromosome of a first chromosome pair with the first foreign nucleic acid sequence and a first chromosome of a second chromosome pair with the second foreign nucleic acid sequence. According to one aspect, the method further includes the step of growing the germline cell into the organism and mating the organism with a wild-type organism of the same species to produce an offspring germline cell including a first chromosome of a first chromosome pair with the first foreign nucleic acid sequence and a first chromosome of a second chromosome pair with the second foreign nucleic acid sequence, and a first chromosome of a third chromosome pair with the third foreign nucleic acid sequence. According to one aspect, the method further includes the step of growing the germline cell into the organism and mating the organism with a wild-type organism of the same species to produce an offspring germline cell including a first chromosome of a first chromosome pair with the first foreign nucleic acid sequence and a first chromosome of a second chromosome pair with the second foreign nucleic acid sequence, a first chromosome of a third chromosome pair with the third foreign nucleic acid sequence and a first chromosome of an Nth chromosome pair with the Nth foreign nucleic acid sequence. According to one aspect, the method further includes the step of growing the germline cell into the organism and mating the organism with a wild-type organism of the same species to produce an offspring germline cell including a first chromosome of a first chromosome pair with the first foreign nucleic acid sequence and the second foreign nucleic acid sequence. According to one aspect, the method further includes the step of growing the germline cell into the organism and mating the organism with a wild-type organism of the same species to produce an offspring germline cell including a first chromosome of a first chromosome pair with the first foreign nucleic acid sequence, the second foreign nucleic acid sequence, and the third nucleic acid sequence. According to one aspect, the method further includes the step of growing the germline cell into the organism and mating the organism with a wild-type organism of the same species to produce an offspring germline cell including a first chromosome of a first chromosome pair with the first foreign nucleic acid sequence, the second foreign nucleic acid sequence, the third nucleic acid sequence, and the Nth nucleic acid sequence. According to one aspect, the method further includes the step of growing the germline cell into the organism and mating the organism with a wild-type organism of the same species to produce an offspring germline cell wherein the first foreign nucleic acid sequence, the second foreign nucleic acid sequence, the third nucleic acid sequence and the Nth foreign nucleic acid sequence are located on the same or a different chromosome. According to one aspect, the second foreign nucleic acid sequence is expressed by the offspring germline cell to produce the RNA guided DNA binding protein nuclease and the one or more RNAs wherein the RNA guided DNA binding protein nuclease and an associated guide RNA co-localize to an associated target location on a second chromosome of the first chromosome pair and the RNA guided DNA binding protein nuclease cleaves the second chromosome at the target location in a cleavage site specific manner and, inserting the first foreign nucleic acid sequence into the second chromosome of the first chromosome pair at the cleavage site to render the offspring germline cell homozygous for the first foreign nucleic acid sequence. According to one aspect, the steps of growing the germline cell into the organism and mating the organism with a wild type organism of the same species is repeated to produce a population of organisms of the same species with increased representation of the first foreign nucleic acid sequence. According to one aspect, the first foreign nucleic acid sequence includes one or more selected gene sequences. According to one aspect, the first foreign nucleic acid sequence includes one or more selected gene sequences and the one or more selected gene sequences is within the genome of the organisms within the population of organisms. According to one aspect, at least one of the target locations are within a nucleic acid sequence important to the fitness of the organism, such as an essential gene, such that a deletion event or a mutation that removes the target location(s) will be highly deleterious and removed from the population by natural selection. According to one aspect, the method further includes the step of growing the germline cell into the organism and mating the organism with a wild-type organism of the same species in a local population of organisms to produce an offspring which further mate with wild-type organisms in the local population of organisms wherein the Cas9 guide RNA system changes the frequency of a specific genome alteration within the local population of organisms. According to one aspect, the method further includes the step of growing the germline cell into the organism and mating the organism with a wild-type organism of the same species in a local population of organisms to produce an offspring which further mate with wild-type organisms in the local population of organisms wherein the Cas9 guide RNA system keeps the frequency of a given genome alteration at a specified level within the local population.

Aspects of the present disclosure include a method of limiting inheritance of a genome editing system having mutually dependent components to a desired fixed number of generations of a target organism including the steps of, in a germline cell, (1) inserting into a first and second chromosome of a first chromosome pair a first nucleic acid encoding a desired cargo gene for insertion into a first target locus and including flanking regions identical to regions on the first target locus, wherein the first nucleic acid sequence optionally includes a Cas9 protein or a guide RNA complementary to the first target locus, (2) inserting into a first and second chromosome of a second chromosome pair a second nucleic acid sequence encoding a guide RNA complementary to the first target locus and/or a Cas9 protein and including flanking regions identical to regions on a second target locus, and (3) inserting into a first and second chromosome of a third chromosome pair a third nucleic acid sequence encoding a guide RNA complementary to the second target locus and/or a Cas9 protein and including flanking regions identical to regions on the second target locus, and (4) inserting into a first and second chromosome of an Nth chromosome pair an Nth nucleic acid sequence encoding a guide RNA and optionally a Cas9 protein and including corresponding promoters, wherein the guide RNA is complementary to a target locus on genomic DNA at an insertion site of an Nth−1 nucleic acid sequence encoding a guide RNA and optionally a Cas9 protein, wherein the first nucleic acid sequence, the second nucleic acid sequence, the third nucleic acid sequence or the Nth nucleic acid sequence are on the same or different chromosomes, wherein the first nucleic acid sequence, the second nucleic acid sequence, the third nucleic acid sequence or the Nth nucleic acid sequence are separated by at least 1000, 2000, 5000, 10000, 100000, 1000000 bases if on the same chromosome pair, further including the step of growing the germline cell into the organism and mating the organism with a wild-type organism of the same species in a local population of organisms to produce an offspring which further mate with wild-type organisms in the local population of organisms until the Cas9 guide RNA system is no longer inherited by progeny.

1. A method of altering a eukaryotic germline cell of an organism comprising
inserting into the first and second chromosomes of a chromosome pair a first foreign nucleic acid sequence including a cargo desired gene and including flanking sequences corresponding to a first target locus, wherein the cargo desired gene is to be inserted into the genome of progeny of the germline cell by an RNA guided DNA binding protein nuclease system,
inserting into the first and second chromosomes of a chromosome pair a second foreign nucleic acid sequence encoding an RNA guided DNA binding protein nuclease and one or more guide RNAs, and including corresponding promoter sequences,
wherein the sites of insertion of the first foreign nucleic acid sequence and the second foreign nucleic acid are separated by at least 1000 bases if on the same chromosome pair,
wherein the one or more guide RNAs encoded by the second foreign nucleic acid sequence are complementary to one or more target locations on genomic DNA at the first target locus which is an insertion site for the first foreign nucleic acid sequence.

2. The method of claim 1 wherein the RNA guided DNA binding protein nuclease is Cas9.

3. The method of claim 1 wherein the RNA guided DNA binding protein nuclease is a Cas9 nickase.

4. The method of claim 1 wherein at least one target location is within an essential gene and the foreign nucleic acid sequence is inserted adjacent to and replacing part of the essential gene.

5. The method of claim 1 wherein the first foreign nucleic acid sequence is inserted by homologous recombination.

6. The method of claim 1 wherein the germline cell is a fungal cell, a plant cell, an insect cell or a mammalian cell.

7. A method of altering a eukaryotic germline cell comprising
(1) inserting into a first and second chromosome a first nucleic acid encoding a guide RNA complementary to a target location on a target locus Y and including flanking regions identical to regions on a target locus X,
(2) inserting into a first and second chromosome a second nucleic acid sequence encoding a guide RNA complementary to a target location on a target locus Z and/or a Cas9 protein and including flanking regions identical to regions on a target locus Y, and
(3) inserting into a first and second chromosome a third nucleic acid sequence encoding a desired cargo gene and including flanking regions identical to regions on a target locus Z, wherein the third nucleic acid sequence encodes Cas9 when the second nucleic acid sequence encodes a guide RNA, wherein the third nucleic acid sequence lacks either Cas9 or a guide RNA when the second nuclease acid sequence encodes both Cas9 and a guide RNA, or wherein the third nucleic acid sequence encodes a guide RNA when the second nucleic acid sequence encodes Cas9,
wherein the first nucleic acid sequence, the second nucleic acid sequence or the third nucleic acid sequence are on the same or different chromosomes,
wherein the first nucleic acid sequence, the second nucleic acid sequence and/or the third nucleic acid sequence are separated by at least 1000 bases if on the same chromosome pair.

8. The method of claim 7 wherein the first nucleic acid sequence, the second nucleic acid sequence and the third nucleic acid sequence include one or more corresponding promoter sequences.

9. The method of claim 7 wherein the Cas9 protein is an enzymatically active Cas9.

10. The method of claim 7 wherein the Cas9 protein is a Cas9 nuclease or a Cas9 nickase.

11. The method of claim 7 wherein the target locus Z is within an essential gene and the desired cargo gene is inserted adjacent to and replacing part of the essential gene.

12. The method of claim 7 wherein the third nucleic acid sequence is inserted by homologous recombination.

13. The method of claim 7 wherein the germline cell is a fungal cell, a plant cell, an insect cell or a mammalian cell.

14. The method of claim 7 wherein inheritance of a genome editing system having mutually dependent components is limited to a desired fixed number of generations of the target organism.

15. A method of altering a eukaryotic germline cell comprising
(1) inserting into a first and second chromosome of a first chromosome pair a first nucleic acid encoding a desired cargo gene for insertion into a first target locus and including flanking regions identical to regions on the first target locus, wherein the first nucleic acid sequence optionally includes a Cas9 protein or a guide RNA complementary to the first target locus,
(2) inserting into a first and second chromosome of a second chromosome pair a second nucleic acid sequence encoding a guide RNA complementary to the first target locus and/or a Cas9 protein and including flanking regions identical to regions on a second target locus, and
(3) inserting into a first and second chromosome of a third chromosome pair a third nucleic acid sequence encoding a guide RNA complementary to the second target locus and/or a Cas9 protein and including flanking regions identical to regions on the second target locus, and
(4) inserting into a first and second chromosome of an Nth chromosome pair an Nth nucleic acid sequence encoding a guide RNA and optionally a Cas9 protein and including corresponding promoters, wherein the guide RNA is complementary to a target locus on genomic DNA at an insertion site of an Nth-1 nucleic acid sequence encoding a guide RNA and optionally a Cas9 protein,
wherein the first nucleic acid sequence, the second nucleic acid sequence, the third nucleic acid sequence or the Nth nucleic acid sequence are on the same or different chromosomes,
wherein the first nucleic acid sequence, the second nucleic acid sequence, the third nucleic acid sequence or the Nth nucleic acid sequence are separated by at least 1000 bases if on the same chromosome pair.

16. The method of claim 15 wherein the first nucleic acid sequence, the second nucleic acid sequence, the third nucleic acid sequence or the Nth nucleic acid sequence include one or more corresponding promoter sequences.

17. The method of claim 15 wherein the Cas9 protein is an enzymatically active Cas9.

18. The method of claim 15 wherein the Cas9 protein is a Cas9 nuclease or a Cas9 nickase.

19. The method of claim 15 wherein the target locus Z is within an essential gene and the desired cargo gene is inserted adjacent to and replacing part of the essential gene.

20. The method of claim 15 wherein the third nucleic acid sequence is inserted by homologous recombination.

21. The method of claim 15 wherein the germline cell is a fungal cell, a plant cell, an insect cell or a mammalian cell.

22. The method of claim 15 further including the step of growing the germline cell into the organism.

23. The method of claim 15 further including the step of growing the germline cell into the organism and mating the organism with a wild-type organism of the same species to produce an offspring germline cell including a first chromosome of a first chromosome pair with the first foreign nucleic acid sequence and a first chromosome of a second chromosome pair with the second foreign nucleic acid sequence.

24. The method of claim 15 further including the step of growing the germline cell into the organism and mating the organism with a wild-type organism of the same species to produce an offspring germline cell including a first chromosome of a first chromosome pair with the first foreign nucleic acid sequence and a first chromosome of a second chromosome pair with the second foreign nucleic acid sequence, and a first chromosome of a third chromosome pair with the third foreign nucleic acid sequence.

25. The method of claim 15 further including the step of growing the germline cell into the organism and mating the organism with a wild-type organism of the same species to produce an offspring germline cell including a first chromosome of a first chromosome pair with the first foreign nucleic acid sequence and a first chromosome of a second chromosome pair with the second foreign nucleic acid sequence, a first chromosome of a third chromosome pair with the third foreign nucleic acid sequence and a first chromosome of an Nth chromosome pair with the Nth foreign nucleic acid sequence.

26. The method of claim 15 further including the step of growing the germline cell into the organism and mating the organism with a wild-type organism of the same species to produce an offspring germline cell including a first chromosome of a first chromosome pair with the first foreign nucleic acid sequence and the second foreign nucleic acid sequence.

27. The method of claim 15 further including the step of growing the germline cell into the organism and mating the organism with a wild-type organism of the same species to produce an offspring germline cell including a first chromosome of a first chromosome pair with the first foreign nucleic acid sequence, the second foreign nucleic acid sequence, and the third nucleic acid sequence.

28. The method of claim 15 further including the step of growing the germline cell into the organism and mating the organism with a wild-type organism of the same species to produce an offspring germline cell including a first chromosome of a first chromosome pair with the first foreign nucleic acid sequence, the second foreign nucleic acid sequence, the third nucleic acid sequence, and the Nth nucleic acid sequence.

29. The method of claim 15 further including the step of growing the germline cell into the organism and mating the organism with a wild-type organism of the same species to produce an offspring germline cell wherein the first foreign nucleic acid sequence, the second foreign nucleic acid sequence, the third nucleic acid sequence and the Nth foreign nucleic acid sequence are located on the same or a different chromosome.

30. The method of claim 23 wherein the second foreign nucleic acid sequence is expressed by the offspring germline cell to produce the RNA guided DNA binding protein nuclease and the one or more RNAs wherein the RNA guided DNA binding protein nuclease and an associated guide RNA co-localize to an associated target location on a second chromosome of the first chromosome pair and the RNA guided DNA binding protein nuclease cleaves the second chromosome at the target location in a cleavage site specific manner and,
inserting the first foreign nucleic acid sequence into the second chromosome of the first chromosome pair at the cleavage site to render the offspring germline cell homozygous for the first foreign nucleic acid sequence.

31. The method of claim 24 wherein the steps of growing the germline cell into the organism and mating the organism with a wild type organism of the same species is repeated to produce a population of organisms of the same species with increased representation of the first foreign nucleic acid sequence.

32. The method of claim 5 wherein the first foreign nucleic acid sequence includes one or more selected gene sequences.

33. The method of claim 26, wherein the first foreign nucleic acid sequence includes one or more selected gene sequences and the one or more selected gene sequences is within the genome of the organisms within the population of organisms.

34. The method of claim 15 wherein at least one of the target locations are within a nucleic acid sequence important to the fitness of the organism, such as an essential gene, such that a deletion event or a mutation that removes the target location(s) will be highly deleterious and removed from the population by natural selection.

35. The method of claim 23 further including the step of growing the germline cell into the organism and mating the organism with a wild-type organism of the same species in a local population of organisms to produce an offspring which further mate with wild-type organisms in the local population of organisms wherein the Cas9 guide RNA system changes the frequency of a specific genome alteration within the local population of organisms.

36. The method of claim 23 further including the step of growing the germline cell into the organism and mating the organism with a wild-type organism of the same species in a local population of organisms to produce an offspring which further mate with wild-type organisms in the local population of organisms wherein the Cas9 guide RNA system keeps the frequency of a given genome alteration at a specified level within the local population.

37. A method of limiting inheritance of a genome editing system having mutually dependent components to a desired fixed number of generations of a target organism comprising, in a germline cell,
(1) inserting into a first and second chromosome of a first chromosome pair a first nucleic acid encoding a desired cargo gene for insertion into a first target locus and including flanking regions identical to regions on the first target locus, wherein the first nucleic acid sequence optionally includes a Cas9 protein or a guide RNA complementary to the first target locus, (2) inserting into a first and second chromosome of a second chromosome pair a second nucleic acid sequence encoding a guide RNA complementary to the first target locus and/or a Cas9 protein and including flanking regions identical to regions on a second target locus, and
(3) inserting into a first and second chromosome of a third chromosome pair a third nucleic acid sequence encoding a guide RNA complementary to the second target locus and/or a Cas9 protein and including flanking regions identical to regions on the second target locus, and
(4) inserting into a first and second chromosome of an Nth chromosome pair an Nth nucleic acid sequence encoding a guide RNA and optionally a Cas9 protein and including corresponding promoters, wherein the guide RNA is complementary to a target locus on genomic DNA at an insertion site of an Nth-1 nucleic acid sequence encoding a guide RNA and optionally a Cas9 protein,
wherein the first nucleic acid sequence, the second nucleic acid sequence, the third nucleic acid sequence or the Nth nucleic acid sequence are on the same or different chromosomes,
wherein the first nucleic acid sequence, the second nucleic acid sequence, the third nucleic acid sequence or the Nth nucleic acid sequence are separated by at least 1000 bases if on the same chromosome pair,
further including the step of growing the germline cell into the organism and mating the organism with a wild-type organism of the same species in a local population of organisms to produce an offspring which further mate with wild-type organisms in the local population of organisms until the Cas9 guide RNA system is no longer inherited by progeny.

38. (canceled)

39. A method of altering a eukaryotic germline cell of an organism comprising
inserting into the first and second chromosomes of a chromosome pair at a first site of insertion Z a first foreign nucleic acid sequence including a cargo desired alteration,
inserting into the first and second chromosomes of a chromosome pair at a second site of insertion Y a second foreign nucleic acid sequence encoding an RNA guided DNA binding protein nuclease with an appropriate promoter sequence and one or more guide RNAs with corresponding promoter sequences and flanking sequences corresponding to the second site of insertion Y,
wherein the sites of insertion Z and Y of the first foreign nucleic acid sequence and the second foreign nucleic acid are separated by at least 1000 bases if on the same chromosome pair,
wherein the one or more guide RNAs encoded by the second foreign nucleic acid sequence are complementary to one or more target locations on genomic DNA at the first site of insertion Z for the first foreign nucleic acid sequence,
wherein the first foreign nucleic acid sequence is to be copied into any corresponding chromosome that 1) initially lacks the first foreign nucleic acid sequence and 2) is inherited by any progeny of the germline cell that also inherits the first and second foreign nucleic acid sequences, wherein if progeny inherit first and second foreign nucleic acid sequences, the first foreign nucleic acid sequence will exhibit drive,
wherein copying is to occur via cutting of the target locations on genomic DNA at the first site of insertion Z by an RNA guided DNA binding protein nuclease system including the RNA guided DNA binding protein nuclease and the one or more guide RNAs complementary to the one or more target locations on genomic DNA at the first site of insertion Z and subsequent repair using the homologous chromosome encoding the first foreign nucleic acid sequence.

40.-49. (canceled)

50. A method of altering a eukaryotic germline cell of an organism comprising
inserting into the first and second chromosomes of a chromosome pair at a first site of insertion Z a first foreign nucleic acid sequence including a cargo desired alteration and encoding an RNA-guided DNA binding protein nuclease with an appropriate promoter sequence,
inserting into the first and second chromosomes of a chromosome pair at a second site of insertion Y a second foreign nucleic acid sequence encoding one or more guide RNAs with corresponding promoter sequences,
wherein the one or more guide RNAs encoded by the second foreign nucleic acid sequence are complementary to one or more target locations on genomic DNA at the first site of insertion Z for the first foreign nucleic acid sequence,
wherein the sites of insertion Z and Y of the first foreign nucleic acid sequence and the second foreign nucleic acid are separated by at least 1000 bases if on the same chromosome pair,
wherein the one or more guide RNAs encoded by the second foreign nucleic acid sequence are complementary to one or more target locations on genomic DNA at the first site of insertion Z for the first foreign nucleic acid sequence,
wherein the first foreign nucleic acid sequence is to be copied into any corresponding chromosome that 1) initially lacks the first foreign nucleic acid sequence and 2) is inherited by any progeny of the germline cell that also inherits the first and second foreign nucleic acid sequences, wherein if progeny inherit the first and second foreign nucleic acid sequences, the first foreign nucleic acid sequence will exhibit drive,
wherein copying is to occur via cutting of the target locations on genomic DNA at the first site of insertion Z by an RNA guided DNA binding protein nuclease system including the RNA guided DNA binding protein nuclease and the one or more guide RNAs complementary to the one or more target locations on genomic DNA at the first site of insertion Z and subsequent repair using the homologous chromosome encoding the first foreign nucleic acid sequence.

51.-71. (canceled)

72. A method of limiting the activity a genome editing system that causes itself and adjacent sequences to be copied into corresponding chromosomes initially lacking said sequences over successive generations by constructing the system as a chain of mutually dependent components wherein the terminal component of the system is not successfully copied in each successive generation, comprising, in a germline cell,
(1) inserting into the first and second chromosomes of a first chromosome pair at a first site of insertion Z a first foreign nucleic acid sequence including a desired DNA alteration,
(2) inserting into the first and second chromosome of a second chromosome pair at a second site of insertion (Z-1) a second nucleic acid sequence encoding one or more guide RNAs complementary to the first site of insertion Z together with corresponding promoter sequences and an RNA guided DNA binding protein nuclease with an appropriate promoter sequence,
(3) inserting into the first and second chromosomes of an Nth chromosome pair an Nth nucleic acid sequence encoding one or more guide RNAs complementary to the (N−1th) site of insertion together with corresponding promoter sequences and optionally including an RNA-guided DNA binding protein nuclease with an appropriate promoter sequence,
wherein no two sites of insertion are separated by less than 1000 bases if located on the same chromosome pair,
further including the step of growing the germline cell into an organism and cross-breeding to generate a population of organisms, releasing a desired number of organisms into the wild where they will mate wild-type organisms of the same species in the local population to produce offspring, where said offspring further mate with other organisms in the local population until the desired DNA alteration is present at a desired level of incidence in the local population but remains substantially absent from more distant populations.

73.-134. (canceled)
